(19) United States
(12) Patent Application Publication
(10) Pub. No.: US 2024/0252652 A1
(43) Pub. Date: Aug. 1, 2024

(54) PROTEINS HAVING UNNATURAL AMINO ACIDS AND METHODS OF USE

(71) Applicant: The Regents of the University of California, Oakland, CA (US)

(72) Inventors: Lei Wang, San Francisco, CA (US); Shanshan Li, San Francisco, CA (US)

(21) Appl. No.: 18/565,032

(22) PCT Filed: Jun. 2, 2022
(86) PCT No.: PCT/US2022/031925

§ 371 Date: Nov. 28, 2023

Related U.S. Application Data
(60) Provisional application No. 63/238,357, filed on Aug. 30, 2021.
(60) Provisional application No. 63/196,006, filed on Jun. 2, 2021.

(51) Int. Cl. A61K 47/54; A61K 38/00; C07D 207/416; C07K 14/705; C12N 9/00; C12Q 1/6886

(52) U.S. Cl. CPC A61K 47/545; C07D 207/416; C07K 14/70596; C12N 9/93; C12Q 1/6886; C12Y 601/01026; A61K 38/00; C12Q 2600/154

(57) ABSTRACT

Provided herein are, inter alia, compounds of Formula (I): biomolecules (e.g., proteins, lipids, RNA, glycans) comprising the compounds; bioconjugates comprising the compounds; processes for preparing the compounds, biomolecules, and bioconjugates; and their uses.

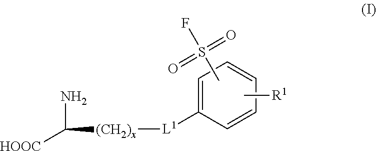

### CROSS-REFERENCE TO RELATED APPLICATIONS

This application claims the benefit of priority to U.S. Application No. 63/238,357 filed Aug. 30, 2021, and U.S. Application No. 63/196,006 filed Jun. 2, 2021, the disclosures of which are incorporated by reference herein in their entirety.

### STATEMENT AS TO RIGHTS TO INVENTIONS MADE UNDER FEDERALLY SPONSORED RESEARCH AND DEVELOPMENT

This invention was made with government support under grant no. R01GM118384 and R01CA258300 awarded by the National Institutes of Health. The government has certain rights in the invention.

### REFERENCE TO A “SEQUENCE LISTING,” A TABLE, OR A COMPUTER PROGRAM LISTING APPENDIX SUBMITTED AS AN ASCII FILE

The Sequence Listing written in file 048536.txt, created 2022, bytes, machine format IBM-PC, MS Windows operating system, is hereby incorporated by reference.

### BACKGROUND

Interactions between glycans and proteins play important biological roles in living systems. Proteins interact with glycans of glycoproteins, glycolipids, and polysaccharides presented on the cell surface to influence biological activity and recognition. (Ref. 1). Protein-glycan interactions are involved in a broad range of biology processes, such as cell-cell communication, organism development, tumor cell metastasis, bacteria and virus invasion, and immune response. (Refs. 2-4). Despite a central role for molecular encounters, protein-glycan interactions are challenging to study due to their dynamic nature, transient interaction, and often large number of interacting partners involved. (Ref 5). Glycan structure is not genetically encoded, making it not amenable to common genetic techniques and difficult to achieve monosaccharide specificity. Another salient feature adding to the difficulty is the generally low affinity of the single protein-glycan interaction, with equilibrium dissociation constant K_{d} most often in the millimolar and some in micromolar range. (Refs. 1, 6). Thus it has been extremely difficult to generate high affinity protein binders for glycans. In particular, many cancer cells overexpress distinct surface glycans and pathogenic microbes are covered with glycans not found in eukaryotic cells, but anti-glycan antibodies with high affinity and specificity remain lacking. (Refs. 4, 7, 8). Therefore, to facilitate basic research of glycobiology and to exploit glycan-based diagnosis and therapy, a general approach is highly desired to increase the affinity of proteins for glycans and to stabilize their transient interactions specifically. The ability to covalently cross-link proteins with glycans under mild cellular and in vivo settings would offer a unique solution to these challenges. The present disclosure is directed to these, as well as other, important ends.

### SUMMARY

Provided herein are compounds of Formula (I), where the substituents are as defined herein:

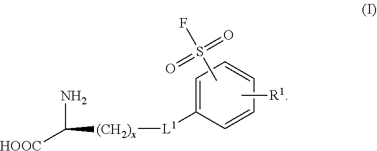

Provided herein are biomolecules comprising an unnatural amino acid, where the unnatural amino comprises a side chain of Formula (II), where the substituents are as defined herein:

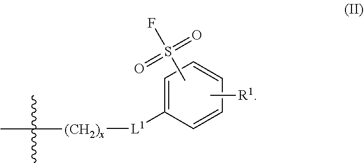

In embodiments, the biomolecule is a lipid, RNA, or glycan. In embodiments, the biomolecule is a protein. In embodiments, the biomolecule is a RNA-binding protein. In embodiments, the biomolecule is a glycan-binding protein. In embodiments, the protein is sialic acid binding Ig like lectin (Siglec) or a sialoglycan binding V-set domain of sialic acid binding Ig like lectin (Siglec).

Provided herein are biomolecule conjugate of Formula (III), where the substituents are as defined herein:

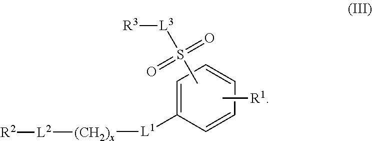

In embodiments, R² is a protein, a lipid, RNA, or a glycan, and R³ is a protein, a lipid, RNA, or a glycan. In embodiments, R² is a protein, and R³ is a protein. In embodiments, R² is a protein, and R³ is a RNA. In embodiments, R² is a protein, and R³ is a mRNA. In embodiments, R² is a RNA-binding protein, and R³ is a RNA. In embodiments. R² is a protein, a lipid, or RNA, and R³ is a glycan. In embodiments, R² is a protein, and R³ is a glycan. In embodiments, R² is a glycan-binding protein, and R³ is a glycan. In embodiments, R² comprises Siglec or a sialoglycan binding V-set domain of Siglec, and R³ comprises a sialoglycan. In embodiments, R³ is bonded to —S(O₂)— via a ribose moiety.

Provided herein are methods of treating cancer in a patient by administering to the patient an effective amount of a protein comprising an unnatural amino acid, wherein the unnatural amino comprises a side chain of Formula (II), and wherein the protein comprises Siglec or a sialoglycan binding V-set domain of Siglec. In embodiments, the cancer has elevated levels of sialoglycan.

These and other embodiments of the disclosure are provided in detail herein.

### BRIEF DESCRIPTION OF THE DRAWINGS

FIGS. 1A-1F. Sulfonyl fluoride was identified suitable for cross-linking glycan through proximity-enabled reactivity using a strategy involving plant-and-cast small molecule cross-linkers. FIG. 1A: Scheme of the strategy: when the plant-and-cast small molecule cross-linker is added to protein-glycan complex, the succinimide ester of the cross-linker reacts rapidly with Lys sidechains of the protein, placing the less reactive test functionality in close proximity to glycan. If the functionality reacts with glycan driven by proximity-enabled reactivity, the glycan will be covalently cross-linked to the protein for detection. FIG. 1B: Chemical structures of five cross-linkers tested to cross-link protein with glycan. FIG. 1C: Function analysis of the refolded Siglec-7v with the glycosphingolipid glycan microarray, confirming Siglec-7v preferably binding with the linear Neu5Acα2-8Neu5 Ac-terminating glycan ligands. FIG. 1D: Chemical structures of azido-GD3 for binding with Siglec-7v and the negative control azido-lac. FIG. 1E: Scheme showing the cross-linking and detection procedures. Siglec-7v was incubated with azido-GD3 for binding, after which the cross-linker was added to crosslink. Biotin was subsequently appended onto azido-GD3 via click chemistry for detection of the crosslinked GD3. FIG. 1F: Among the five tested cross-linkers, only NHSF cross-linked Siglec-7v with azido-GD3.

FIGS. 2A-2D. Cross-linking of Siglec-7v with azido-GD3 by NHSF was dependent on concentration and the specific protein-glycan binding. Top panels are western blots for GD3 via detection of biotin; bottom panels are western blots for Siglec-7v via detection of its C-terminal His×6 tag. FIG. 2A: Cross-linking was dependent on the presence of Siglec-7v, azido-GD3, and NHSF. FIG. 2B: Cross-linking was dependent on the concentration of azido-GD3. FIG. 2C: Cross-linking was dependent on the specific binding of azido-GD3 to Siglec-7v. When azido-lac was used with Siglec-7v and NHSF, a faint background band running below the cross-linking band was detectable, which also appeared in Siglec-7v plus NHSF (no azido-GD3) or Siglec-7v plus azido-GD3 (no NHSF). FIG. 2D: Cross-linking was dependent on the concentration of NHSF. Faint background bands in the anti-biotin blots were due to low level reaction of alkyne-biotin with protein Siglec-7v nonspecifically, a common background when using azide-alkyne for click labeling.

FIGS. 3A-3D. Cross-linking site on Siglec-7v and distance dependence of the cross-linker indicate that sulfonyl fluoride of NHSF reacted with glycan via proximity-enabled reactivity. FIG. 3A: Crystal structure of Siglec-7v binding with α(2,8)-disialygangioside GT1b (PDB: 2HRL). NHSF cross-linking site, Lys127, on Siglec-7v is shown in magenta stick. All other Lys sites are shown in grey stick. FIG. 3B: NHSF cross-linking of azido-GD3 with Siglec-7v Lys to Gly mutants. FIG. 3C: Structures of NHSF analogs with different linker lengths. FIG. 3D: Cross-linking of Siglec-7v with azido-GD3 with the NHSF analogs. Faint background bands in the anti-biotin blots were due to low level reaction of alkyne-biotin with protein Siglec-7v nonspecifically, a common background when using azide-alkyne for click labeling.

FIGS. 4A-4G. Genetic incorporation of SFY into proteins in E. coli. FIG. 4A: Structure of SFY. FIG. 4B: Amino acid sequences of the evolved MmSFYRS, MaSFYRS, and the corresponding WT PylRS. FIG. 4C: Western blot analysis of SFY incorporation into sfGFP(2TAG) by Mm-tRNA^{Pyl}/MmSFYRS pair. FIG. 4D: ESI-TOF MS spectrum of intact sfGFP(2SFY) protein expressed by Mm-tRNA^{Pyl}/MmSFYRS pair. FIG. 4E: Tandem MS spectrum of Z(24SFY) expressed by Mm-tRNA^{Pyl}/MmSFYRS pair. U represents SFY. FIG. 4F: Western blot analysis of SFY incorporation into sfGFP(2TAG) by Ma-tRNA^{Pyl}/MaSFYRS pair. FIG. 4G: ESI-TOF MS spectrum of intact sfGFP(2SFY) protein expressed by Ma-tRNA^{Pyl}/MaSFYRS pair.

FIGS. 5A-5F. Siglec-7(SFY) cross-linked with azido-GD3 in vitro and with sialoglycan on cell surface. FIG. 5A: ESI-MS spectrum of intact Siglec-7v(104SFY) confirmed SFY incorporation. FIG. 5B: Cross-linking of azido-GD3 with Siglec-7v with SFY incorporated at indicated Lys sites. FIG. 5C: Siglec-7(SFY) cross-linked with azido-GD3 but not azido-lac. FIG. 5D: Crystal structure of Siglec-7v in complex of GT1b (PDB: 2HRL), showing Lys104. Lys127, and Gln 129 in magenta stick, at which SFY incorporation led to cross-linking of azido-GD3. FIGS. 5E-5F: Flow cytometric quantification of Siglec-7v protein bound on SK-MEL-5 cell surface. After washing, more Siglec-7v(127SFY) bound with sialoglycan on SK-MEL-5 cell surface than WT Siglec-7v (FIG. 5E), but there was no binding difference when the cells were pretreated with sialidase to remove cell surface sialoglycan (FIG. 5F). The line and error bar represent mean±SD; n=3 independent batches of Siglec-7v proteins. *** p<0.001; multiple t test. NS: not significant.

FIGS. 6A-6D. Siglec-7v(SFY) enhanced NK cell killing of cancer cells. FIG. 6A: Scheme showing the use of Siglec-7v(127SFY) to block the interaction between sialoglycan on tumor cell surface and Siglec-7 of NK cells. Decreasing the inhibitory signal of Siglec-7 on NK cells would enhance NK killing of tumor cells. FIGS. 6B-6D: Cytotoxicity assay of three hypersialylated cancer cell lines showed that Siglec-7v(127SFY) enhanced NK-92 cell killing over the WT Siglec-7v. The line and error bar represent mean±SD; n=3 independent batches of Siglec-7v proteins. * p<0.05; ** p<0.01; *** p<0.001; multiple t test.

FIG. 7. Chemo-enzymatic synthesis of azido-lactose and azido-GD3.

FIGS. 8A-8B show a comparison of SFY incorporation into different sites of GFP using Mm-tRNA^{Pyl}/MmSFYRS and Ma-tRNA^{Pyl}/MaSFYRS in E. coli. Fluorescence intensities of the expressed sfGFP (2SFY). EGFP (40SFY), and EGFP (182SFY) in E. coli cells using the indicated tRNA^{Pyl} and SFYRS were quantified with flow cytometry. In all cases, the WT-MaPylT and MaSFYRS pair afforded the highest incorporation efficiency of SFY in E. coli.

FIG. 9 shows the primers for cloning described in the example.

FIG. 10 show the name and structure of the 58 glycans on the glycan microarray described in the example.

FIGS. 11A-11H show that genetically encoding SFY allows crosslinking of His, Tyr, Lys residues in protein and of RNA in cells. FIG. 11A: Structure of SFY. FIG. 11B: Fluorescence confocal images HEK293 cells expressing EGFP(40TAG) gene and the Mm-tRNA^{Pyl}/MmSFYRS with and without 1 mM SFY. FIG. 11C: Flow cytometric analysis of SFY incorporation into EGFP(40TAG) in HEK293 cells using Ma-tRNA^{Pyl}/MaSFYRS. FIG. 11D: Structure of Afb-Z complex showing two proximal sites for SFY and target residue X incorporation. FIG. 11E: Analysis of crosslinking of Afb(24SFY) with MBP-Z(7X) in E. coli cells. Left: Western blot of E. coli cell lysate; Right: SDS-PAGE of proteins His-tag purified from E. coli. Maltose binding protein (MBP) was fused to the N-terminus of Z protein to better separate Z from Afb in size. FIG. 11F: Crystal structure of E. coli GST (PDB: 1A0F) showing site 103 and 107 at the dimer interface. FIG. 11G: Western blot analysis of lysate of HEK293T cells expressing GST(103SFY-107X). X is the target residue indicated. FIG. 11H: Western blot analysis E. coli cells expressing Hfq with SFY incorporated at site 25 or 49. Cell lysate samples were treated with or without RNase before loading, and an anti-His antibody was used to detect the 6×His tag appended at the C-terminus of expressed Hfq. Star indicates a cross-linked band.

FIGS. 12A-12B show design of GRIP-seq for in vivo detection of m6A on RNA with single-nucleotide resolution. FIG. 12A: Scheme showing the principle of using GRIP-seq to detect RNA modifications in vivo, using m6A as an example. A reader protein recognizing the RNA modification is expressed in cells, with a latent bioreactive Uaa (SFY) incorporated near the recognition site to cross-link bound RNA for identification. This is followed by partial RNase digestion and an immunoprecipitation enriching reader-proteins and their cross-linked RNA fragments. After dephosphorylation and 3′ adaptor ligation with RNA fragments, the cross-linked protein-RNA are separated by SDS-PAGE and transferred to a nitrocellulose membrane. The membrane regions above the read-protein (75 kDa above) are excised and treated with proteinase K to release the cross-linked RNA fragments. The released RNA fragments are further prepared into libraries for pair-end high-throughput sequencing. In the final libraries, read 2 begins with a random-mer sequence (random 10mer, added with 3′ cDNA adaptor ligation) followed by the sequence corresponding to the 3′ end of reverse-transcribed cDNA, the junction of which indicates the cross-link sites causing the revers-transcription termination (See materials and methods). FIG. 12B: Structure of YTH domain (from human YTHDF1) binding with m6A nucleotide (PDB: 4RCJ). Tyr397, the site chosen for incorporation of SFY is shown in grey stick. RNA is colored in yellow and YTH protein in green.

FIGS. 13A-13B are glow cytometric analysis of SFY incorporation into EGFP in HEK293 cells. FIG. 13A: SFY incorporation into EGFP(182TAG) in HEK293 cells using Ma-tRNA^{Pyl}/MaSFYRS. FIG. 13B: SFY incorporation into EGFP(40TAG) or EGFP(182TAG) in HEK293 cells using Mm-tRNA^{Pyl}/MmSFYRS.

FIG. 14 shows a cell viability assay for HEK293T incubated with various concentrations of SFY for 24 h or 48 h. Error bars represent s.e.m.; n=3 independent tests.

FIGS. 15A-15C provide m6A data. FIG. 15A: Western blot analysis demonstrating the successful expression and immunoprecipitation of YTH-WT and YTH-397SFY proteins in HEK293 cells. An anti-HA antibody was used for detection. FIG. 15B: Agarose gel analysis of PCR products from YTH GRIP PCR for regions of JUN (upper right). ACTB (lower left), and DICER1 (lower right) mRNAs. FIG. 15C: m6A sites identified from YTH GRIP for region of ACTB and DICER1 mRNAs. ▾ triangles showed ligation sites of sequenced clones from YTH-397SFY expressing cells. Arrows showed the m6A site indicated from sequenced clone results. ▴ triangles showed m6A site reported from previous study. (Tang et al. Nucleic Acids Res. 49:D134-D143 (2020)).

FIGS. 16A-16B show the addition of 3′-sialyllactose did not reduce the cross-linking of Siglec-7v(127FSY) with azido-GD3. FIG. 16A: Structure of 3′-Sialyllactose. FIG. 16B: The addition of 3′-Sialyllactose didn't reduce the cross-linking of Siglec-7v(127SFY) with azido-GD3. Siglec-7v(127SFY) (60 μM) was incubated with 2 mM azido-GD3, then supplemented without or with different concentrations of 3′-sialyllactose. Samples are boiled and subjected for Western blot analysis.

FIGS. 17A-17D provide a comparative study between NHSF pretreated Siglec-7v (Siglec-7v-SF) and Siglec-7v(127SFY). FIG. 17A: Siglec-7v(127SFY) cross-linked azido-GD3 efficiently, while Siglec-7v-SF could not. Siglec-7v(127SFY) or Siglec-7v-SF was incubated with azido-GD3 or azido-lac followed with Western blot detection. The azido group was click reacted with alkyne-biotin for Western blot detection of GD3/lac. FIGS. 17B-17C: Siglec-7v(127SFY) bound to the surface of BT20 (FIG. 17B) and SK-MEL-28 (FIG. 17C) cell lines in a dose-dependent manner, while Siglec-7v-SF could not bind with either cells. Cells were treated with protein, washed, stained with a fluorescently labeled antibody specific for the His×6 tag appended at the C-terminus of Siglec-7v, and quantified with flow cytometry. FIG. 17D: Siglec-7v(127SFY) significantly enhanced NK cell killing of cancer cells, while Siglec-7v-SF could not. Pre-stained BT-20 cells were incubated with 12 μM Siglec-7v-SF or Siglec-7v(127SFY) for 2 h. Cells were then washed and incubated with NK-92 cells for 4 h. Cells were stained with propidium iodide and NK cytotoxicity was evaluated by flow cytometry. Control group: no protein treatment. The line and error bar represent mean±SEM; n=3 independent batches of Siglec-7v proteins. *** p<0.001; NS, not significant, multiple t test.

FIG. 18 is a glycan microarray analysis of Fc-Siglec-7 commercially available from R&D Systems (Minneapolis, MN). Siglec-7v purified from E. coli showed specific signals in binding with all sialoglycans that are known binders of Siglec 7, including G20 and G28 (FIG. 1C). The commercial Fc-Siglec-7 expressed in mouse cells showed a similar binding pattern but had no signal for G20 and G28, possibly because of glycosylation effect and/or interference of the Fc tag.

FIGS. 19A-19K providing regarding detection of endogenous m6A sites in mammalian cells throughout the transcriptome using high-throughput sequencing. FIG. 19A: Western blot analysis demonstrating the successful expression and immunoprecipitation of YTH-WT and YTH-397SFY proteins in HEK293 cells. An anti-HA antibody was used for detection.

FIG. 19B: Individual GRIP-seq IP samples were analyzed for the numbers of peaks identified per gene. Pearson correlation coefficients (r values in the figure) indicated a high degree of overlap between YTH-397SFY IP replicates. FIG. 19C: The most enriched motifs found in peak regions from individual GRIP-seq IP samples. The enriched DRACH motifs in YTH-397SFY-IP samples were identical to the published m6A consensus motif. FIG. 19D: Histogram of the nucleotide compositions at the cross-linking sites from YTH-397SFY replicates. Y-axis: the numbers of reads corresponding to RNAs cross-linked at different nucleotides in YTH-397SFY IP replicates. FIG. 19E: Scheme of the m6A site identification using individual YTH GRIP for specific RNA regions. FIG. 19F: Agarose gel analysis of PCR products from individual YTH GRIP PCR for regions of JUN (left), and DICER1 (right) mRNAs. FIGS. 19G-19H: Genome browser tracks of alignments of sanger-sequenced clones from individual YTH GRIP, and GRIP-seq data in JUN (FIG. 19G) and DICER1 (FIG. 19H) mRNA regions. Red triangles showed ligation sites of sequenced clones from YTH-397SFY expressing cells. Known m6A sites from published datasets (Tang et al. Nucleic Acids Res. 49:D134-D143 (2020)) were marked as grey triangles, m6A sites identified from GRIP-seq were marked as yellow triangles. FIG. 19I: The most enriched motif found in peak regions of novel m6A sites from GRIP-seq. The enriched DRACH motif was identical to the published m6A consensus motif. FIG. 19J: Peak regions of novel m6A sites from GRIP-seq showed metagene distribution profiles typical for m6A. FIG. 19K: The predicted minimum folding free energy (MFE) was plotted for regions surrounding m6A sites from datasets of GRIP-seq. DART-seq, and published m6A sites (from m6A-atlas). Tang et al. Nucleic Acids Res. 49: D134-D143 (2020); Meyer. Nat. Methods 16:1275-1280 (2019). A sliding window with 30-nt in length and a step of 3-nt was used to calculate MFE. For each window, the central position was used for alignment. A minus position value indicates upstream of m6A sites, whereas a positive value indicates downstream of m6A sites. Notably, a lower MFE value indicates a higher potential for RNA secondary structures.

FIGS. 20A-20E show GRIP-seq in vivo detected m6A on RNA with single-nucleotide resolution in mammalian cells. FIG. 20A: The most enriched motif found in GRIP-seq data of YTH-397SFY-IP samples. The enriched DRACH motif was identical to the published m6A consensus motif. FIG. 20B: Reverse-transcription-termination sites identified from YTH-397SFY IP samples showed metagene distribution profiles typical for m6A. FIG. 20C: Genome browser tracks of GRIP-seq data in JUN and DICER1 mRNA regions. Reverse-transcription-termination sites (RT-termination sites) from GRIP-seq were marked as yellow triangles. Known m6A sites from published datasets were marked as grey triangles. Tang et al. Nucleic Acids Res. 49:D134-D143 (2020). FIG. 20D: Plot showing the cross-links enriched at the upstream of the DRACH motif. X-axis indicated the position relative to m6A (0) position) in the DRACH motif. Y-axis indicated the read numbers (representing RNA molecules) of cross-links at the corresponding positions from YTH-397SFY IP samples. FIG. 20E: Pie chart showing the nucleotide composition at the cross-linking sites. FIG. 20F: Violin plot (with box plot inside) presenting the distribution of RNA abundance of the two gene groups: genes containing only novel m6A sites (salmon, N=1,699), and genes containing only known m6A sites (grey, N=1,826). Y-axis: TPM (Transcript per million reads) values in log 10 scale, representing the RNA abundance. TPM values of each gene in HEK293T cells were from Protein Atlas database. Uhlen et al, Science, 347:1260419 (2015). Two-sided Wilcox rank-sum test for the RNA abundance of the two gene groups: **** p<0.0001 (p=4.645×10⁻¹⁵).

### DETAILED DESCRIPTION

### Definitions

Unless defined otherwise, technical and scientific terms used herein have the same meaning as commonly understood by a person of ordinary skill in the art. See, e.g., Singleton et al., Dictionary of Microbiology and Molecular Biology, 2nd ed., J. Wiley & Sons (New York, NY 1994); Sambrook et al., Molecular Cloning, A Laboratory Manual, Cold Springs Harbor Press (Cold Springs Harbor, NY 1989). Any methods, devices and materials similar or equivalent to those described herein can be used in the practice of this disclosure. The following definitions are provided to facilitate understanding of certain terms used frequently herein and are not meant to limit the scope of the present disclosure.

“Nucleic acid” refers to nucleotides (e.g., deoxyribonucleotides or ribonucleotides) and polymers thereof in either single-, double- or multiple-stranded form, or complements thereof. The terms “polynucleotide,” “oligonucleotide,” “oligo” or the like refer, in the usual and customary sense, to a linear sequence of nucleotides. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g., polynucleotides contemplated herein include any types of RNA, e.g., mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphorothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, Oligonucleotides and Analogues: A Practical Approach, Oxford University Press) as well as modifications to the nucleotide bases such as in 5-methyl cytidine or pseudouridine and peptide nucleic acid backbones and linkages. Other analog nucleic acids include those with positive backbones: non-ionic backbones, modified sugars, and non-ribose backbones (e.g., phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, Glycan Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanidine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “amino acid side chain” refers to the functional substituent contained on amino acids. For example, an amino acid side chain may be the side chain of a naturally occurring amino acid. Naturally occurring amino acids are those encoded by the genetic code (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine), as well as those amino acids that are later modified, e.g., hydroxyproline, γ-carboxyglutamate, and O-phosphoserine. In embodiments, the amino acid side chain is a non-natural amino acid side chain. In embodiments, the amino acid side chain is H,

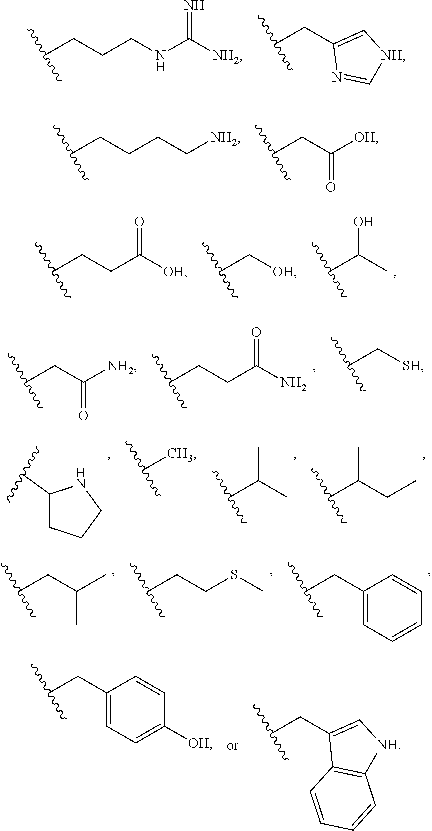

The term “non-natural amino acid side chain” or “unnatural amino acid side chain” refers to the functional substituent of compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium, allylalanine, 2-aminoisobutryric acid. Non-natural amino acids are non-proteinogenic amino acids that either occur naturally or are chemically synthesized. Such analogs have modified R groups (e.g., norleucine) or modified peptide backbones, but retain the same basic chemical structure as a naturally occurring amino acid. Non-limiting examples include exo-cis-3-aminobicyclo[2.2.1]hept-5-ene-2-carboxylic acid hydrochloride, cis-2-aminocycloheptane-carboxylic acid hydrochloride, cis-6-amino-3-cyclohexene-1-carboxylic acid hydrochloride, cis-2-amino-2-methylcyclohexanecarboxylic acid hydrochloride, cis-2-amino-2-methylcyclopentane-carboxylic acid hydrochloride, 2-(Boc-aminomethyl)benzoic acid, 2-(Boc-amino)octanedioic acid, Boc-4,5-dehydro-Leu-OH (dicyclohexylammonium), Boc-4-(Fmoc-amino)-L-phenylalanine, Boc-β-Homopyr-OH, Boc-(2-indanyl)-Gly-OH, 4-Boc-3-morpholineacetic acid, 4-Boc-3-morpholine acetic acid, Boc-pentafluoro-D-phenylalanine, Boc-pentafluoro-L-phenylalanine, Boc-Phe(2-Br)—OH, Boc-Phe(4-Br)—OH, Boc-D-Phe(4-Br)—OH, Boc-D-Phe(3-Cl)—OH, Boc-Phe(4-NH2)-OH, Boc-Phe(3-NO2)-OH, Boc-Phe(3,5-F2)-OH, 2-(4-Boc-piperazino)-2-(3,4-dimethoxy-phenyl)acetic acid purum, 2-(4-Boc-piperazino)-2-(2-fluorophenyl)acetic acid purum, 2-(4-Boc-piperazino)-2-(3-fluorophenyl)acetic acid purum, 2-(4-Boc-piperazino)-2-(4-fluorophenyl)acetic acid purum, 2-(4-Boc-piperazino)-2-(4-methoxyphenyl)acetic acid purum, 2-(4-Boc-piperazino)-2-phenylacetic acid purum, 2-(4-Boc-piperazino)-2-(3-pyridyl)acetic acid purum, 2-(4-Boc-piperazino)-2-[4-(trifluoromethyl)phenyl]acetic acid purum, Boc-β-(2-quinolyl)-Ala-OH, N-Boc-1,2,3,6-tetrahydro-2-pyridinecarboxylic acid, Boc-β-(4-thiazolyl)-Ala-OH, Boc-β-(2-thienyl)-D-Ala-OH, Fmoc-N-(4-Boc-aminobutyl)-Gly-OH, Fmoc-N-(2-Boc-aminoethyl)-Gly-OH, Fmoc-N-(2,4-dimethoxy benzyl)-Gly-OH, Fmoc-(2-indanyl)-Gly-OH, Fmoc-pentafluoro-L-phenylalanine, Fmoc-Pen(Trt)-OH, Fmoc-Phe(2-Br)—OH, Fmoc-Phe(4-Br)—OH, Fmoc-Phe(3,5-F2)-OH, Fmoc-β-(4-thiazolyl)-Ala-OH, Fmoc-β-(2-thienyl)-Ala-OH, 4-(Hydroxymethyl)-D-phenylalanine.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following groups each contain amino acids that are conservative substitutions for one another: (1) Alanine (A), Glycine (G); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); (6) Phenylalanine (F), Tyrosine (Y), Tryptophan (W); (7) Serine (S), Threonine (T); and (8) Cysteine (C), Methionine (M).

The terms “polypeptide,” “peptide” and “protein” are used interchangeably herein to refer to a polymer of amino acid residues, wherein the polymer may in embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

An amino acid residue in a protein “corresponds” to a given residue when it occupies the same essential structural position within the protein as the given residue. For example, a selected residue in a selected protein corresponds to Lysine127 of Siglec-7 when the selected residue occupies the same essential spatial or other structural relationship as Lysine127 in Siglec-7. In embodiments, where a selected protein is aligned for maximum homology with Siglec-7, the position in the aligned selected protein aligning with Lysine127 is said to correspond to Lysine127. Instead of a primary sequence alignment, a three dimensional structural alignment can also be used, e.g., where the structure of the selected protein is aligned for maximum correspondence with Siglec-7 and the overall structures compared. In this case, an amino acid that occupies the same essential position as Lysine127 in the structural model is said to correspond to the Lysine127 residue. Thus, Lysine127 of SEQ ID NO: 1 corresponds to Lysine127 of SEQ ID NOS:2-4 (which can alternatively be referred to as Lysine86 in SEQ ID NO:2; as Lysine87 in SEQ ID NO:3; and as Lysine86 in SEQ ID NO:4).

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, or at least 65%, 70%, 75%, 80%, 85%, 90%, 91%, 92%, 93%, 94%, 95%, 96%, 97%, 98%, 99% identity over a specified region, when compared and aligned for maximum correspondence over a comparison window or designated region) as measured using a BLAST or BLAST 2.0 sequence comparison algorithms with default parameters described below, or by manual alignment and visual inspection (e.g., NCBI web site ncbi.nlm.nih.gov/BLAST/ or the like). Such sequences are then said to be “substantially identical,” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

The term “biomolecule” as used herein refers to large macromolecules such as, for example, proteins, glycans, lipids, and nucleic acids, as well as small molecules such as, for example, primary and secondary metabolites. In embodiments, the term biomolecule refers to a protein. In embodiments, the term biomolecule refers to a glycan. In embodiments, the term biomolecule refers to RNA.

The term “biomolecule moiety” as used herein refers to biomolecules, including large macromolecules such as, for example, proteins, glycans, lipids, and nucleic acids (e.g., RNA), as well as small molecules such as, for example, primary and secondary metabolites. Thus, in embodiments, the biomolecule moiety is a peptidyl moiety, a glycan moiety, a lipid moiety or a nucleic acid moiety. Biomolecule moieties may form part of a molecule (e.g., biomolecule). For example, biomolecule moieties may form part of a biomolecule conjugate, where the biomolecule conjugate includes two or more biomolecule moieties. In embodiments, the biomolecule conjugate includes two or more biomolecule moieties conjugated via a bioconjugate linker.

The term “glycan” or “carbohydrate” as used herein refers to compounds containing monosaccharides linked glycosidically (e.g., N-linked, O-linked). Monosaccharides generally contain from about three to about nine carbon atoms. Exemplary monosaccharides include glyceraldehyde-3-phosphate, erythrose, threose, erythrulose, ribose, deoxyribose, arabinose, lyxose, xylose, ribulose, xylulose, glucose, mannose, galactose, gulose, idose, talose, allose, altrose, fructose, piscose, sorbose, tagatose, glycer-D-manno-heptose, seduhelpulose, methylthiolincos amide, neuraminic acid, sialic acid, legionaminic acid, psudaminic acid, and the like. In embodiments, the term “glycan” refers to a compound comprising a ribose.

The term “glycan moiety” refers to a monovalent radical of a glycan. The glycan moiety may be substituted with additional chemical moieties. In embodiments, the glycan moiety is bonded (covalently or non-covalently) with a protein, a lipid, a glycan, or RNA. In embodiments, the glycan moiety is associated with (e.g., on the surface of or embedded within the surface membrane) a cancer cell. In embodiments, the glycan moiety is covalently bonded via a ribose moiety with a protein, a lipid, a glycan, or RNA. In embodiments, the glycan moiety is covalently bonded via a ribose moiety with a protein.

The term “peptidyl moiety” refers to a protein, protein fragment, or peptide. The peptidyl moiety may be substituted with additional chemical moieties. In embodiments, a peptidyl moiety is a monovalent radical of a protein.

The term “lipid moiety” refers to a lipid or lipid fragment. The lipid may be substituted with additional chemical moieties. In embodiments, a lipid moiety is a monovalent radical of a lipid.

The term “RNA moiety” refers to a RNA, as described herein. In embodiments, a RNA moiety is a monovalent radical of RNA. In embodiments, RNA moiety refers to mRNA. In embodiments, a mRNA moiety is a monovalent radical of mRNA.

The term “pyrrolysyl-tRNA synthetase” refers to an enzyme (including homologs, isoforms, and functional fragments thereof) with pyrrolysyl-tRNA synthetase activity. Pyrrolysyl-tRNA synthetase is an aminoacyl-tRNA synthetase that catalyzes the reaction necessary to attach α-amino acid pyrrolysine to the cognate tRNA (tRNA^{pyl}), thereby allowing incorporation of pyrrolysine during proteinogenesis at amber stop codons (i.e., UAG). The term includes any recombinant or naturally-occurring form of pyrrolysyl-tRNA synthetase or variants, homologs, or isoforms thereof that maintain pyrrolysyl-tRNA synthetase activity (e.g. within at least 30%, 40%, 50%, 60%, 70%, 80%, 90%, 95%, or 100% activity compared to wild-type pyrrolysyl-tRNA synthetase). In embodiments, the variants, homologs, or isoforms have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring pyrrolysyl-tRNA synthetase. In embodiments, the mutant pyrrolysyl-tRNA synthetase catalyzes the attachment of the compound of Formula (I) to a tRNA^{pyl}.

The terms “tRNA^{Pyl}” and “rTNA^{Pyl}_{CUA}” and “tRNA^{Pyl}_{CUA}” (i.e., tRNA(superscript Pyl)(subscript CUA)) are used interchangeably and all refer to a single-stranded RNA molecule containing about 70 to 90 nucleotides which fold via intrastrand base pairing to form a characteristic cloverleaf structure that carries a specific amino acid (e.g., compound of Formula (I)) and matches it to its corresponding codon (i.e., a complementary to the anticodon of the (RNA) on an mRNA during protein synthesis. In tRNA^{Py}, the anticodon is CUA. Anticodon CUA is complementary to amber stop codon UAG. The abbreviation “Pyl” of tRNA^{Py} stands for pyrrolysine and the “CUA” of tRNA^{Py} refers to its anticodon CUA. In embodiments, tRNA^{Py} is attached to the compound of Formula (I).

The term “substrate-binding site” as used herein refers to residues located in the enzyme active site that form temporary bonds or interactions with the substrate. In embodiments, the substrate-binding site of pyrrolysyl-tRNA synthetase refers to residues located in the active site of pyrrolysyl-tRNA synthetase that form temporary bonds or interactions with the amino acid substrate.

The term “vector” refers to a nucleic acid molecule capable of transporting another nucleic acid to which it has been linked. One type of vector is a “plasmid”, which refers to a linear or circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors,” In general, expression vectors of utility in recombinant DNA techniques are often in the form of plasmids. The terms “plasmid” and “vector” can be used interchangeably as the plasmid is the most commonly used form of vector. However, the disclosure is intended to include such other forms of expression vectors, such as viral vectors (e.g., replication defective retroviruses, adenoviruses and adeno-associated viruses), which serve equivalent functions. Some viral vectors are capable of targeting a particular cells type either specifically or nonspecifically. Exemplary vectors that can be used include, but are not limited to, pEvol vector, pMP vector, pET vector, pTak vector, pBad vector.

The term “complex” refers to a composition that includes two or more components, where the components bind together to make a functional unit. In embodiments, a complex described herein include a mutant pyrrolysyl-tRNA synthetase described herein and an amino acid substrate (e.g., the compound of Formula (I)). In embodiments, a complex described herein includes a mutant pyrrolysyl-tRNA synthetase described herein and a tRNA (e.g., tRNA^{Py}). In embodiments, a complex described herein includes a mutant pyrrolysyl-tRNA synthetase described herein, an amino acid substrate (e.g., SFY) and a tRNA (e.g., tRNA^{Py}). In embodiments, a complex described herein includes at least two components selected from the group consisting of a mutant pyrrolysyl-tRNA synthetase described herein, an amino acid substrate (e.g., the compound of Formula (I)), a polypeptide containing the compound of Formula (I), and a tRNA (e.g., tRNA^{Py}).

The term “protein complex” refers to a composition that includes two or more proteins, where the proteins are proximal to each other but not bound together: the proteins are covalently bound together; or the proteins are ionically bound together. In embodiments, the proteins are proximal to each other but not bound together. In embodiments, the proteins are covalently bonded together. In embodiments, proteins are ionically bonded together. In embodiments, the proteins are covalently and ionically bonded together. In embodiments, a first protein in the protein complex comprises compound of Formula (I), and a second protein in the protein complex comprises serine, threonine, or a combination thereof. In embodiments, the compound of Formula (I) in the first protein is proximal to the serine and/or threonine in the second protein. In embodiments “proximal” means that the compound of Formula (I) in the first protein and the serine and/or threonine in the second protein are close enough to each other for a chemical reaction to occur between the compound of Formula (I) and the serine and/or threonine. In embodiments, the chemical reaction is a SuFEx reaction.

The term “glycan-binding protein/glycan complex” refers to a composition that includes at least one glycan-binding protein and at least one glycan, where the glycan-binding protein and glycan are proximal to each other but not bound together: the glycan-binding protein and glycan are covalently bound together; or the glycan-binding protein and glycan are ionically bound together. In embodiments, the glycan-binding protein and glycan are proximal to each other but not bound together. In embodiments, the glycan-binding protein and glycan are covalently bonded together. In embodiments, the glycan-binding protein and glycan are covalently bonded together via ribose moiety in the glycan. In embodiments, glycan-binding protein and glycan are ionically bonded together. In embodiments, the protein and glycan are covalently and ionically bonded together. In embodiments, the glycan-binding protein comprises the compound of Formula (I), and the glycan comprises a hydroxyl moiety. In embodiments, the compound of Formula (I) in the glycan-binding protein is proximal to the hydroxyl moiety in the glycan. In embodiments “proximal” means that the compound of Formula (I) in the glycan-binding protein and the hydroxyl moiety in the glycan are close enough to each other for a chemical reaction to occur between the compound of Formula (I) and the hydroxyl moiety in the glycan. In embodiments, the chemical reaction is a SuFEx reaction.

The term “RNA-binding protein/RNA complex” refers to a composition that includes at least one RNA-binding protein and at least one RNA, where the RNA-binding protein and RNA are proximal to each other but not bound together; the RNA-binding protein and RNA are covalently bound together; or the RNA-binding protein and RNA are ionically bound together. In embodiments, the RNA-binding protein and RNA are proximal to each other but not bound together. In embodiments, the RNA-binding protein and RNA are covalently bonded together. In embodiments. RNA-binding protein and RNA are ionically bonded together. In embodiments, the protein and RNA are covalently and ionically bonded together. In embodiments, the RNA-binding protein comprises the compound of Formula (I), and the RNA comprises a hydroxyl moiety or a N⁶-methyladenosine moiety. In embodiments, the compound of Formula (I) in the RNA-binding protein is proximal to the RNA. In embodiments “proximal” means that the compound of Formula (I) in the RNA-binding protein and the RNA are close enough to each other for a chemical reaction to occur between the compound of Formula (I) and the RNA. In embodiments, the chemical reaction is a SuFEx reaction.

The terms “transfection”, “transduction”, “transfecting” or “transducing” can be used interchangeably and are defined as a process of introducing a nucleic acid molecule or a protein to a cell. Nucleic acids are introduced to a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. In embodiments, the nucleic acid molecules are introduced into a cell using electroporation following standard procedures well known in the art. For viral-based methods of transfection any useful viral vector may be used in the methods described herein. Examples for viral vectors include, but are not limited to retroviral, adenoviral, lentiviral and adeno-associated viral vectors. In embodiments, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

The term “isolated,” when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

“Contacting” is used in accordance with its plain ordinary meaning and refers to the process of allowing at least two distinct species (e.g. chemical compounds including glycans, RNA, amino acids, proteins, peptides, biomolecules, or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture. The term “contacting” may include allowing two species to react, interact, or physically touch, wherein the two species may be biomolecule moieties as described herein. In some embodiments, contacting includes allowing two proteins, a protein and a glycan, or a protein and RNA, as described herein to interact.

The symbol “ [symbol] ” or “-” denotes the point of attachment of a chemical moiety to the remainder of a molecule or chemical formula.

The compounds described herein may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds described herein, whether radioactive or not, are encompassed within the scope of the present disclosure.

“Analog,” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

A “detectable agent” or “detectable moiety” is a compound or composition detectable by appropriate means such as spectroscopic, photochemical, biochemical, immunochemical, chemical, magnetic resonance imaging, or other physical means. In embodiments, the compounds described herein comprise a detectable agent. For example, useful detectable agents include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y. ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g., fluorescent dyes), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monocrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g., carbon-11, nitrogen-13, oxygen-15, fluorine-18, rubidium-82), fluorodeoxyglucose (e.g., fluorine-18 labeled), any gamma ray emitting radionuclides, positron-emitting radionuclide, radiolabeled glucose, radiolabeled water, radiolabeled ammonia, biocolloids, microbubbles (e.g. including microbubble shells including albumin, galactose, lipid, and/or polymers; microbubble gas core including air, heavy gases, perfluorcarbon, nitrogen, octafluoropropane, perflexane lipid microsphere, perflutren, etc.), iodinated contrast agents (e.g., iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide. A detectable moiety is a monovalent detectable agent or a detectable agent capable of forming a bond with another compound or composition.

Radioactive substances (e.g., radioisotopes) that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y. ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³P, ¹²⁴P, ¹²⁵P, ¹³¹P, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac. Paramagnetic ions that may be used as additional imaging agents in accordance with the embodiments of the disclosure include, e.g., ions of transition and lanthanide metals (e.g., metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu. In embodiments, the compounds described herein comprise a radioisotope.

The term “sulfur-fluoride exchange reaction” or “SuFEx” refers to a type of click chemistry as described in detail by, e.g., Dong et al, Angewandte Chemie, 53(36):9340-9448 (2014); and Wang et al, J. Am. Chem. Soc., 140(15):4995-4999 (2018). The term “proximally-enabled” SuFEx refers to the sulfur-fluoride exchange reaction occurring when the reactive species are proximal to each other, i.e., spatially close enough for the SuFEx reaction to occur. The proximity may occur within a single biomolecule (e.g., protein) or between two different biomolecules (e.g., protein and glycan). The skilled artisan could readily determine whether the reactive species are sufficiently proximal for the reaction to occur, e.g., sulfur-fluoride exchange reaction between the compound of Formula (I) and glycan (e.g., a hydroxyl group on a glycan).

In embodiments, “proximal” means that two compounds (e.g., biomolecules, proteins, peptides, amino acids, glycans) are adjacent (e.g., but not covalently bonded together). In embodiments, “proximal” means up to about 25 angstroms. In embodiments, “proximal” means up to about 20 angstroms. In embodiments, “proximal” means up to about 15 angstroms. In embodiments, “proximal” means up to about 10 angstroms. In embodiments, “proximal” means from about 1 angstrom to about 25 angstroms. In embodiments, “proximal” means from about 1 angstrom to about 20 angstroms. In embodiments, “proximal” means from about 1 angstrom to about 15 angstroms. In embodiments, “proximal” means from about 1 angstrom to about 12 angstroms. In embodiments, “proximal” means from about 1 angstrom to about 10 angstroms. In embodiments, “proximal” means from about 1 angstrom to about 8 angstroms. In embodiments, “proximal” means from about 1 angstrom to about 6 angstroms. In embodiments, “proximal” means from about 1 angstrom to about 5 angstroms. In embodiments, “proximal” means from about 1 angstroms to about 4 angstroms.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds.

The term “alkylene,” by itself or as part of another substituent, means, unless otherwise stated, a divalent radical derived from an alkyl, as exemplified by, e.g., —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH—CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom. A heteroalkyl moiety may include two optionally different heteroatoms. A heteroalkyl moiety may include three optionally different heteroatoms. A heteroalkyl moiety may include four optionally different heteroatoms. A heteroalkyl moiety may include five optionally different heteroatoms. A heteroalkyl moiety may include up to 8 optionally different heteroatoms. The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′- and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl.” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperavinyl, and the like. A “cycloalkylene” and a “heterocycloalkylene,” alone or as part of another substituent, means a divalent radical derived from a cycloalkyl and heterocycloalkyl, respectively.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. In embodiments, bridged monocyclic rings contain a monocyclic cycloalkyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. In embodiments, fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkyl is attached to the parent molecular moiety through any carbon atom contained within the monocyclic cycloalkyl ring. In embodiments, cycloalkyl groups are optionally substituted with one or two groups which are independently oxo or thia. In embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted by one or two groups which are independently oxo or thia. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkyl is attached to the parent molecular moiety through any carbon atom contained within the base ring. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic cycloalkyl groups include, but are not limited to tetradecahydrophenanthrenyl, perhydrophenothiazin-1-yl, and perhydrophenoxazin-1-yl.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. In embodiments, monocyclic cycloalkenyl ring systems are cyclic hydrocarbon groups containing from 3 to 8 carbon atoms, where such groups are unsaturated (i.e., containing at least one annular carbon carbon double bond), but not aromatic. Examples of monocyclic cycloalkenyl ring systems include cyclopentenyl and cyclohexenyl. In embodiments, bicyclic cycloalkenyl rings are bridged monocyclic rings or a fused bicyclic rings. In embodiments, bridged monocyclic rings contain a monocyclic cycloalkenyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic cycloalkenyls include, but are not limited to, norbornenyl and bicyclo[2.2.2]oct 2 enyl. In embodiments, fused bicyclic cycloalkenyl ring systems contain a monocyclic cycloalkenyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkenyl is attached to the parent molecular moiety through any carbon atom contained within the monocyclic cycloalkenyl ring. In embodiments, cycloalkenyl groups are optionally substituted with one or two groups which are independently oxo or thia. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkenyl is attached to the parent molecular moiety through any carbon atom contained within the base ring. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a monocyclic heteroaryl, a monocyclic cycloalkyl, a monocyclic cycloalkenyl, and a monocyclic heterocyclyl.

In embodiments, a heterocycloalkyl is a heterocyclyl. The term “heterocyclyl” as used herein, means a monocyclic, bicyclic, or multicyclic heterocycle. The heterocyclyl monocyclic heterocycle is a 3, 4, 5, 6 or 7 membered ring containing at least one heteroatom independently selected from the group consisting of O, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The heterocyclyl monocyclic heterocycle is connected to the parent molecular moiety through any carbon atom or any nitrogen atom contained within the heterocyclyl monocyclic heterocycle. Representative examples of heterocyclyl monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperavinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The heterocyclyl bicyclic heterocycle is a monocyclic heterocycle fused to either a phenyl, a monocyclic cycloalkyl, a monocyclic cycloalkenyl, a monocyclic heterocycle, or a monocyclic heteroaryl. The heterocyclyl bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-1H-indolyl, and octahydrobenzofuranyl. In embodiments, heterocyclyl groups are optionally substituted with one or two groups which are independently oxo or thia. In certain embodiments, the bicyclic heterocyclyl is a 5 or 6 membered monocyclic heterocyclyl ring fused to a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thia. Multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. The multicyclic heterocyclyl is attached to the parent molecular moiety through any carbon atom or nitrogen atom contained within the base ring. In embodiments, multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic heterocyclyl groups include, but are not limited to 10H-phenothiazin-10-yl, 9,10-dihydroacridin-9-yl, 9,10-dihydroacridin-10-yl, 10H-phenoxazin-10-yl, 10,11-dihydro-5H-dibenzo[b,f]azepin-5-yl, 1,2,3,4-tetrahydropyrido[4,3-g]isoquinolin-2-yl, 12H-benzo[b]phenoxazin-12-yl, and dodecahydro-1H-carbazol-9-yl.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridavinyl, triavinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substituents described herein.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocyclic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” or “-” denotes the point of attachment of a chemical moiety to the remainder of a molecule or chemical formula.

The term “oxo,” as used herein, means an oxygen that is double bonded to a carbon atom.

The term “alkylsulfonyl,” as used herein, means a moiety having the formula —S(O₂)—R′, where R′ is a substituted or unsubstituted alkyl group as defined above. R′ may have a specified number of carbons (e.g., “C₁-C₄ alkylsulfonyl”).

The term “alkylarylene” as an arylene moiety covalently bonded to an alkylene moiety (also referred to herein as an alkylene linker). In embodiments, the alkylarylene group has the formula:

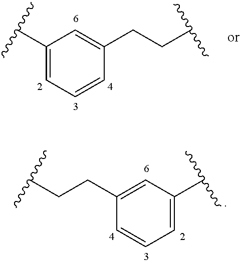 [SMILES: *CCc1cccc(*)c1 ; *CCc1cccc(*)c1]

An alkylarylene moiety may be substituted (e.g. with a substituent group) on the alkylene moiety or the arylene linker (e.g. at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃—SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In embodiments, the ring-forming substituents are attached to adjacent members of the base structure. For example, two ring-forming substituents attached to adjacent members of a cyclic base structure create a fused ring structure. In embodiments, the ring-forming substituents are attached to a single member of the base structure. For example, two ring-forming substituents attached to a single member of a cyclic base structure create a spirocyclic structure. In embodiments, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′— (C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl, heteroalkyl, cycloalkyl, heterocycloalkyl, aryl, heteroaryl, substituted with at least one substituent selected from:
(i) oxo, halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(ii) alkyl, heteroalkyl, cycloalkyl, heterocycloalkyl, aryl, heteroaryl, substituted with at least one substituent selected from: (a) oxo, halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and (b) alkyl, heteroalkyl, cycloalkyl, heterocycloalkyl, aryl, heteroaryl, substituted with at least one substituent selected from: oxo, halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In embodiments, each substituted group described in the compounds herein is substituted with at least one substituent group. More specifically, in embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In embodiments, at least one or all of these groups are substituted with at least one size-limited substituent group. In embodiments, at least one or all of these groups are substituted with at least one lower substituent group.

In embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

Certain compounds of the present disclosure possess asymmetric carbon atoms (optical or chiral centers) or double bonds: the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers. As used herein, the term “isomers” refers to compounds having the same number and kind of atoms, and hence the same molecular weight, but differing in respect to the structural arrangement or configuration of the atoms. The term “tautomer,” as used herein, refers to one of two or more structural isomers which exist in equilibrium and which are readily converted from one isomeric form to another. It will be apparent to one skilled in the art that certain compounds of this disclosure may exist in tautomeric forms, all such tautomeric forms of the compounds being within the scope of the disclosure. Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

“Analog,” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Where a moiety is substituted with an R substituent, the group may be referred to as “R-substituted,” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R³ substituents are present, each R³ substituent may be distinguished as R^{3A}, R^{3B}, wherein each of R^{3A}, R^{3B}, is defined within the scope of the definition of R³ and optionally differently.

A person of ordinary skill in the art will understand when a variable (e.g., moiety or linker) of a compound or of a compound genus (e.g., a genus described herein) is described by a name or formula of a standalone compound with all valencies filled, the unfilled valence(s) of the variable will be dictated by the context in which the variable is used. For example, when a variable of a compound as described herein is connected (e.g., bonded) to the remainder of the compound through a single bond, that variable is understood to represent a monovalent form (i.e., capable of forming a single bond due to an unfilled valence) of a standalone compound (e.g., if the variable is named “methane” in an embodiment but the variable is known to be attached by a single bond to the remainder of the compound, a person of ordinary skill in the art would understand that the variable is actually a monovalent form of methane, i.e., methyl or —CH₃). Likewise, for a linker variable (e.g., L¹, L², or L³ as described herein), a person of ordinary skill in the art will understand that the variable is the divalent form of a standalone compound (e.g., if the variable is assigned to “PEG” or “polyethylene glycol” in an embodiment but the variable is connected by two separate bonds to the remainder of the compound, a person of ordinary skill in the art would understand that the variable is a divalent (i.e., capable of forming two bonds through two unfilled valences) form of PEG instead of the standalone compound PEG).

The term “bond” or “bonded” refers to direct bonds, such as covalent bonds (e.g., direct or a linking group), or indirect bonds, such as non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions, and the like).

The terms “bioconjugate” and “bioconjugate linker” refers to the resulting association between atoms or molecules of “bioconjugate reactive groups” or “bioconjugate reactive moieties”. The association can be direct or indirect. For example, a conjugate between a first bioconjugate reactive group (e.g., —NH2, —C(O)OH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g. a first linker of second linker), or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). In embodiments, bioconjugates or bioconjugate linkers are formed using bioconjugate chemistry (i.e. the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, Advanced Organic Chemistry, 3rd Ed., John Wiley & Sons, New York, 1985; Hermanson, Bioconjugate Techniques, Academic Press, San Diego, 1996; and Feeney et al, Modification of Proteins, Advances in Chemistry Series, Vol. 198, American Chemical Society, Washington, D.C., 1982. In embodiments, the first bioconjugate reactive group (e.g., unnatural amino acid side chain) is covalently attached to the second bioconjugate reactive group (e.g., a hydroxyl group).

“Siglec” or “sialic-acid-binding immunoglobulin-like lectin” refers to a subset of 1-type lectins that bind to sialoglycans and are predominantly expressed on cells of the hematopoietic system in a manner dependent on cell type and differentiation. Whereas sialic acid is ubiquitously expressed, typically at the terminal position of glycoproteins and lipids, only specific, distinct sialoglycan structures are recognized by individual Siglec receptors, depending on identity and linkage to subterminal carbohydrate moieties. Siglecs are generally divided into two groups, a first subset made up of Siglec-1, Siglec-2, Siglec-4 and Siglec-15, and the CD33-related group of Siglecs which includes Siglec-3, Siglec-5, Siglec-6, Siglec-7, Siglec-8, Siglec-9, Siglec-10, Siglec-11, Siglec-12, Siglec-14 and Siglec-16.

“Siglec-7” or “CD328” is a type I trans-membrane protein belonging to the human CD33-related Siglec receptors, is characterized by a sialic acid binding N-terminal V-set Ig domain, two C2-set Ig domains and an intracytoplasmic region containing one immune-receptor tyrosine based inhibitory motif (ITIM) and one ITIM-like motif. Siglec-7 is constitutively expressed on NK cells, dendritic cells, monocytes and neutrophils. The extracellular domain of this receptor preferentially binds a (2,8)-linked disialic acids and branched a 2,6-sialyl residues, such as those displayed by ganglioside GD3.

### Compounds

Provided herein are biomolecules formed through the interaction of latent bioreactive unnatural amino acids with naturally occurring amino acids. The compound of Formula (I), a bioreactive unnatural amino acid, facilitates formation of chemically reactive amino acids with proximal target amino acid residues (e.g., lysine, arginine) by undergoing a click chemistry reaction (e.g., sulfur-fluoride exchange reaction (SuFEx)). For example, the compound of Formula (I) may be inserted into or replace an amino acid in a naturally occurring protein, thereby endowing the protein with the ability to form a chemically reactive amino acid with proximally positioned target functional groups (e.g., a hydroxyl group in a glycan) or amino acid residues (e.g., serine, threonine) with other proteins. The compound of Formula (I) may be used to facilitate the formation of chemically reactive amino acids in proteins and within proteins in both in vitro and in vivo conditions. As such, the bioreactive unnatural amino acid of Formula (I) is useful for forming chemically reactive amino acid residues that can be further chemically modified, as desired.

The compound of Formula (I) has shown excellent chemical functionality (i.e., superior properties) compared to previously described bioreactive unnatural amino acids. For example, the compound of Formula (I) is stable, nontoxic and nonreactive inside cells, yet when placed in proximity to target amino acid residues or reactive moieties (e.g., a hydroxyl group in a glycan) it becomes reactive under cellular conditions. The compound of Formula (I) is able to react with target amino acid residues or other reactive moieties (e.g., a hydroxyl group in a glycan) with great selectivity via proximity-enabled SuFEx reaction within and between proteins and glycans under physiological conditions.

Provided herein are compounds of Formula (I):

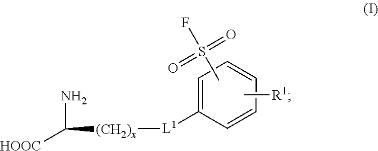

wherein R¹, L¹, and x are as defined herein. In embodiments, the compound of Formula (I) is referred to as an unnatural amino acid.

In embodiments, the compound of Formula (I) is a compound of Formula (IA):

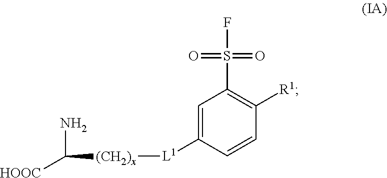

wherein R¹, L¹, and x are as defined herein.

In embodiments, the compound of Formula (I) is a compound of Formula (IB):

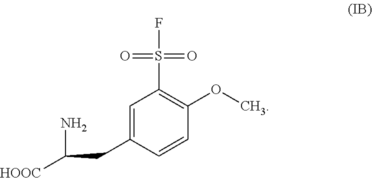 [SMILES: COc1ccc(C[C@H](N)C(=O)O)cc1S(=O)(=O)F]

In embodiments, the compound of Formula (IB) is referred to as SFY.

Provided herein are biomolecules comprising an unnatural amino acid, wherein the unnatural amino comprises a side chain of Formula (II):

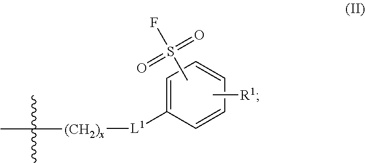

wherein R¹, L¹, and x are as defined herein. In embodiments, the biomolecules are proteins, lipids, RNA, or glycans. In embodiments, the biomolecule is a lipid. In embodiments, the biomolecule is RNA. In embodiments, the biomolecule is a glycan. In embodiments, the biomolecule is a protein.

Provided herein are proteins comprising an unnatural amino acid, wherein the unnatural amino comprises a side chain of Formula (II):

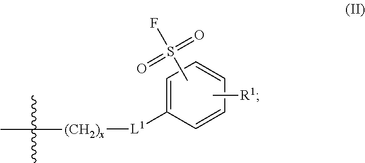

wherein R¹, L¹, and x are as defined herein. In embodiments, the protein comprising the unnatural amino acid comprises a RNA-binding protein. In embodiments, the protein comprising the unnatural amino acid comprises a N⁶-methyladenosine reader protein. In embodiments, the protein comprising the unnatural amino acid comprises a N⁶-methyladenosine demethylase protein. In embodiments, the protein comprising the unnatural amino acid comprises a glycan-binding protein. In embodiments, the protein comprising the unnatural amino acid comprises Siglec. In embodiments, the protein comprising the unnatural amino acid comprises Siglec-1, Siglec-2, Siglec-3, Siglec-4, Siglec-5, Siglec-6, Siglec-7, Siglec-8, Siglec-9, Siglec-10, Siglec-11, Siglec-12, Siglec-14, or Siglec-15. In embodiments, the protein comprising the unnatural amino acid comprises Siglec-1. In embodiments, the protein comprising the unnatural amino acid comprises Siglec-2. In embodiments, the protein comprising the unnatural amino acid comprises Siglec-3 In embodiments, the protein comprising the unnatural amino acid comprises Siglec-4. In embodiments, the protein comprising the unnatural amino acid comprises Siglec-5. In embodiments, the protein comprising the unnatural amino acid comprises Siglec-6. In embodiments, the protein comprising the unnatural amino acid comprises Siglec-8. In embodiments, the protein comprising the unnatural amino acid comprises Siglec-9. In embodiments, the protein comprising the unnatural amino acid comprises Siglec-10. In embodiments, the protein comprising the unnatural amino acid comprises Siglec-11. In embodiments, the protein comprising the unnatural amino acid comprises Siglec-12. In embodiments, the protein comprising the unnatural amino acid comprises Siglec-14. In embodiments, the protein comprising the unnatural amino acid comprises Siglec-15. In embodiments, the protein comprising the unnatural amino acid comprises Siglec-7. In embodiments, the protein comprising the unnatural amino acid comprises Siglec-7 (e.g., SEQ ID NO:1, including embodiments as described herein). In embodiments, the protein comprising the unnatural amino acid comprises a glycan binding V-set domain of a glycan. In embodiments, the protein comprising the unnatural amino acid comprises a sialoglycan binding V-set domain of a Siglec. In embodiments, the protein comprising the unnatural amino acid comprises a sialoglycan binding V-set domain of Siglec-1, Siglec-2, Siglec-3, Siglec-4, Siglec-5, Siglec-6, Siglec-7, Siglec-8, Siglec-9, Siglec-10, Siglec-11, Siglec-12, Siglec-14, or Siglec-15. In embodiments, the protein comprising the unnatural amino acid comprises a sialoglycan binding V-set domain of Siglec-1. In embodiments, the protein comprising the unnatural amino acid comprises a sialoglycan binding V-set domain of Siglec-2. In embodiments, the protein comprising the unnatural amino acid comprises a sialoglycan binding V-set domain of Siglec-3. In embodiments, the protein comprising the unnatural amino acid comprises a sialoglycan binding V-set domain of Siglec-4. In embodiments, the protein comprising the unnatural amino acid comprises a sialoglycan binding V-set domain of Siglec-5. In embodiments, the protein comprising the unnatural amino acid comprises a sialoglycan binding V-set domain of Siglec-6. In embodiments, the protein comprising the unnatural amino acid comprises a sialoglycan binding V-set domain of Siglec-8. In embodiments, the protein comprising the unnatural amino acid comprises a sialoglycan binding V-set domain of Siglec-9. In embodiments, the protein comprising the unnatural amino acid comprises a sialoglycan binding V-set domain of Siglec-10. In embodiments, the protein comprising the unnatural amino acid comprises a sialoglycan binding V-set domain of Siglec-11. In embodiments, the protein comprising the unnatural amino acid comprises a sialoglycan binding V-set domain of Siglec-12. In embodiments, the protein comprising the unnatural amino acid comprises a sialoglycan binding V-set domain of Siglec-14. In embodiments, the protein comprising the unnatural amino acid comprises a sialoglycan binding V-set domain of Siglec-15. In embodiments, the protein comprising the unnatural amino acid comprises a sialoglycan binding V-set domain of Siglec-7 (e.g., SEQ ID NO:2, SEQ ID NO:3, or SEQ ID NO:4, including embodiments as described herein). In embodiments, the term “sialoglycan binding V-set domain” is equivalent to the term “sialoglycan binding domain.”

In embodiments, the unnatural amino comprises a side chain of Formula (II) is an unnatural amino acid side chain of Formula (IIA):

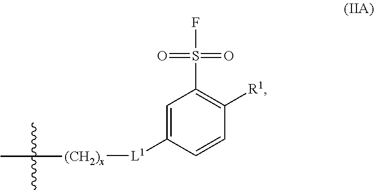

wherein R¹, L¹, and x are as defined herein. In embodiments, the protein is a protein as described for Formula (II), e.g., RNA-binding protein, glycan-binding protein, Siglec, Siglec-1, Siglec-2, Siglec-3, Siglec-4, Siglec-5, Siglec-6, Siglec-7, Siglec-8, Siglec-9, Siglec-10, Siglec-11, Siglec-12, Siglec-14, or Siglec-15; a glycan binding domain of a glycan-binding protein; or a sialoglycan binding V-set domain of Siglec, Siglec-1, Siglec-2, Siglec-3, Siglec-4, Siglec-5, Siglec-6, Siglec-7, Siglec-8, Siglec-9, Siglec-10, Siglec-11, Siglec-12, Siglec-14, or Siglec-15, and all embodiments thereof as described for Formula (II).

In embodiments, the unnatural amino comprises a side chain of Formula (II) is an unnatural amino acid side chain of Formula (IIB):

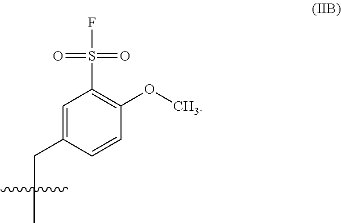 [SMILES: *Cc1ccc(OC)c(S(=O)(=O)F)c1]

In embodiments, the protein is a protein as described for Formula (II), e.g., RNA-binding protein, glycan-binding protein, Siglec, Siglec-1, Siglec-2, Siglec-3, Siglec-4, Siglec-5, Siglec-6, Siglec-7, Siglec-8, Siglec-9, Siglec-10, Siglec-11, Siglec-12, Siglec-14, or Siglec-15; a glycan binding domain of a glycan-binding protein, or a sialoglycan binding V-set domain of Siglec, Siglec-1, Siglec-2, Siglec-3, Siglec-4, Siglec-5, Siglec-6, Siglec-7, Siglec-8, Siglec-9, Siglec-10, Siglec-11, Siglec-12, Siglec-14, or Siglec-15, and all embodiments thereof as described for Formula (II).

Provided herein are biomolecule conjugates of Formula (III):

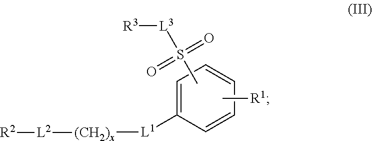

where R¹, R², R³, L¹, L², L³, and x are as defined herein.

In embodiments, the biomolecule conjugate of Formula (III) is a biomolecule conjugate of Formula (IIIA):

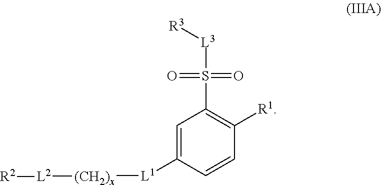

where R¹, R², R³, L¹, L², L³, and x are as defined herein.

In embodiments, the biomolecule conjugate of Formula (III) is a biomolecule conjugate of Formula (IIIB):

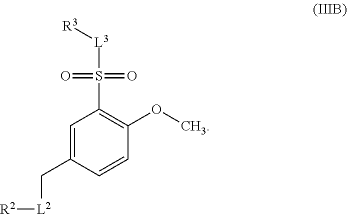

where R², R³, L², and L³ are as defined herein.

Provided herein are compounds of Formula (IV):

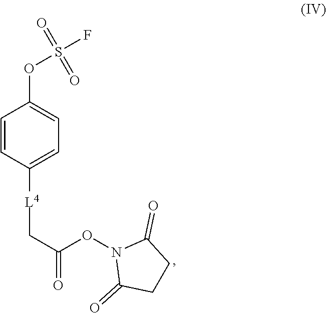

where L⁴ is as defined herein.

In embodiments, the compound of Formula (IV) is a compound of Formula (IVA):

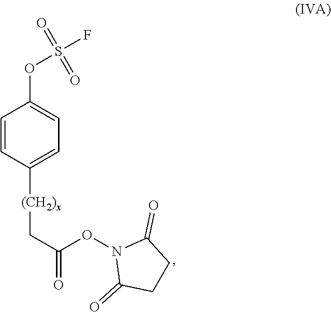

wherein x is as defined herein.

In embodiments, the compound of Formula (IV) is NHFS:

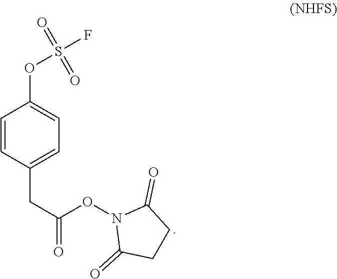 [SMILES: O=C(Cc1ccc(OS(=O)(=O)F)cc1)ON1C(=O)CCC1=O]

Provided herein are compounds of Formula (V):

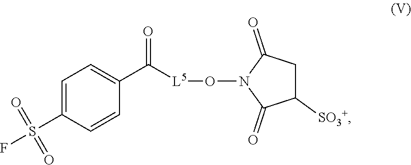

where L⁵ is as defined herein.

With reference to the compounds described herein, x is an integer from 0 to 8. In embodiments, x is an integer from 1 to 8. In embodiments, x is an integer from 1 to 7. In embodiments, x is an integer from 1 to 6. In embodiments, x is an integer from 1 to 5. In embodiments, x is an integer from 1 to 4. In embodiments, x is an integer from 1 to 3. In embodiments, x is an integer of 1 or 2. In embodiments, x is 1. In embodiments, x is 2. In embodiments, x is 3. In embodiments, x is 4. In embodiments, x is 5. In embodiments, x is 6. In embodiments, x is 7. In embodiments, x is 8. In embodiments, x is 0.

With reference to the compounds described herein, R¹ is halogen, —CX¹₃, —CHX¹₂, —CH₂X¹, —OCX¹₃, —OCH₂X¹, —OCHX¹₂, —CN, —SOₙ₁R^{1A}, —SOᵥ₁NR^{1A}R^{1B}, —NHC(O)NR^{1A}R^{1B}, —N(O)ₘ₁, —NR^{1A}R^{1B}, —C(O)R^{1A}, —C(O)—OR^{1A}, —C(O)NR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, substituted or unsubstituted alkyl, or substituted or unsubstituted heteroalkyl. In embodiments, R¹ is halogen, —CX¹₃, —CHX¹₂, —CH₂X¹, —OCX¹₃, —OCH₂X¹, —OCHX¹₂, —CN, —SOₙ₁R^{1A}, —SOᵥ₁NR^{1A}R^{1B}, —NHC(O)NR^{1A}R^{1B}, —N(O)ₘ₁, —NR^{1A}R^{1B}, —C(O)R^{1A}, —C(O)—OR^{1A}, —C(O)NR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, or substituted or unsubstituted heteroalkyl. In embodiments, R¹ is halogen, —CX¹₃, —CHX¹₂, —CH₂X¹, —OCX¹₃, —OCH₂X¹, —OCHX¹₂, —CN, —SOₙ₁R^{1A}, —SOᵥ₁NR^{1A}R^{1B}, —NHC(O)NR^{1A}R^{1B}, —N(O)ₘ₁, —NR^{1A}R^{1B}, —C(O)R^{1A}, —C(O)—OR^{1A}, —C(O)NR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, or unsubstituted heteroalkyl. In embodiments, R¹ is —CN, —SOₙ₁R^{1A}, —SOᵥ₁NR^{1A}R^{1B}, —NHC(O)NR^{1A}R^{1B}, —N(O)ₘ₁, —NR^{1A}R^{1B}, —C(O)R^{1A}, —C(O)—OR^{1A}, —C(O)NR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, or unsubstituted heteroalkyl. In embodiments, R¹ is —CN, —NHC(O)NR^{1A}R^{1B}, —N(O)ₘ₁, —NR^{1A}R^{1B}, —C(O)R^{1A}, —C(O)—OR^{1A}, —C(O)NR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, or unsubstituted heteroalkyl. In embodiments, the alkyl is a C₁₋₄ alkyl. In embodiments, R¹ is substituted or unsubstituted heteroalkyl. In embodiments, R¹ is unsubstituted heteroalkyl. In embodiments, R¹ is unsubstituted 2 to 8 membered heteroalkyl. In embodiments, R¹ is unsubstituted 2 to 6 membered heteroalkyl. In embodiments, R¹ is unsubstituted 2 to 4 membered heteroalkyl. In embodiments, R¹ is —O—(CH₂)ₘCH₃, and m is an integer from 0 to 6. In embodiments, R¹ is —O—(CH₂)ₘCH₃, and m is an integer from 0 to 4. In embodiments, R¹ is —O—(CH₂)ₘCH₃, and m is an integer from 0 to 3. In embodiments, R¹ is —O—(CH₂)ₘCH₃, and m is an integer from 0 to 2. In embodiments, R¹ is —O—(CH₂)ₘCH₃, and m is 0 or 1. In embodiments, R¹ is —O—CH₃. In embodiments. R¹ is —O—CH₂CH₃, In embodiments, R¹ is —O—(CH₂)₂CH₃, In embodiments, R¹ is —O—(CH₂)₃CH₃.

With reference to the compounds described herein, R¹ is ortho, para, or meta to the —S(═O)₂F group. In embodiments, R¹ is ortho to the —S(═O)₂F group. In embodiments, R¹ is para to the —S(═O)₂F group. In embodiments, R¹ is meta to the —S(═O)₂F group.

With reference to the compounds described herein, R^{1A} is hydrogen, substituted or unsubstituted alkyl, or substituted or unsubstituted heteroalkyl. In embodiments, R^{1A} is hydrogen, unsubstituted alkyl, or unsubstituted heteroalkyl. In embodiments, R^{1A} is hydrogen, substituted or unsubstituted C₁₋₄ alkyl, or substituted or unsubstituted 2 to 4 membered heteroalkyl. In embodiments, R^{1A} is hydrogen, unsubstituted C₁₋₄ alkyl, or unsubstituted 2 to 4 membered heteroalkyl. In embodiments, R^{1A} is hydrogen. In embodiments, R^{1A} is unsubstituted C₁₋₄ alkyl. In embodiments, R^{1A} is unsubstituted 2 to 4 membered heteroalkyl.

With reference to the compounds described herein, R^{1B} is hydrogen, substituted or unsubstituted alkyl, or substituted or unsubstituted heteroalkyl. In embodiments, R^{1B} is hydrogen, unsubstituted alkyl, or unsubstituted heteroalkyl. In embodiments, R^{1B} is hydrogen, substituted or unsubstituted C₁₋₄ alkyl, or substituted or unsubstituted 2 to 4 membered heteroalkyl. In embodiments, R^{1B} is hydrogen, unsubstituted C₁₋₄ alkyl, or unsubstituted 2 to 4 membered heteroalkyl. In embodiments, R^{1B} is hydrogen. In embodiments, R^{1B} is unsubstituted C₁₋₄ alkyl. In embodiments, R^{1B} is unsubstituted 2 to 4 membered heteroalkyl.

With reference to the compounds described herein, X¹ is independently —F, —Cl, —Br, or —I. In embodiments, X¹ is independently —F or —Cl. In embodiments, X¹ is —F. In embodiments, X¹ is —Cl. In embodiments, X¹ is —Br. In embodiments, X¹ is —I.

With reference to the compounds described herein, n1 is an integer from 0 to 4. In embodiments n1 is an integer from 0 to 3. In embodiments n1 is an integer from 0 to 2. In embodiments n1 is 0. In embodiments n1 is 1. In embodiments n1 is 2. In embodiments n1 is 3. In embodiments n1 is 4.

With reference to the compounds described herein, m1 is 1 or 2. In embodiments, m1 is 1. In embodiments, m1 is 2.

With reference to the compounds described herein, v1 is 1 or 2. In embodiments, v1 is 1. In embodiments, v1 is 2.

With reference to the compounds described herein, L¹ is a bond, substituted or unsubstituted alkylene, or substituted or unsubstituted heteroalkylene. In embodiments, L¹ is a bond. In embodiments, L¹ is substituted or unsubstituted alkylene. In embodiments, L¹ is substituted or unsubstituted C₁₋₆ alkylene. In embodiments, L¹ is substituted or unsubstituted C₁₋₄ alkylene. In embodiments, L¹ is substituted or unsubstituted heteroalkylene. In embodiments, L¹ is substituted or unsubstituted 2 to 8 membered heteroalkylene. In embodiments, L¹ is substituted or unsubstituted 2 to 6 membered heteroalkylene. In embodiments, L¹ is —NH—C(O)—(CH₂)_{y}— or —NH—C(O)—O—(CH₂)_{y}—, and y is an integer from 0 to 6. In embodiments, L¹ is —NH—C(O)—(CH₂)_{y}— or —NH—C(O)—O—(CH₂)_{y}—, and y is an integer from 0 to 5. In embodiments, L¹ is —NH—C(O)—(CH₂)_{y}— or —NH—C(O)—O—(CH₂)_{y}—, and y is an integer from 0 to 4. In embodiments, L¹ is —NH—C(O)—(CH₂)_{y}— or —NH—C(O)—O—(CH₂)_{y}—, and y is an integer from 0 to 3. In embodiments, L¹ is —NH—C(O)—(CH₂)_{y}— or —NH—C(O)—O—(CH₂)_{y}—, and y is an integer from 0 to 2. In embodiments, L¹ is —NH—C(O)—(CH₂)_{y}—, and y is an integer from 0 to 3. In embodiments, L¹ is —NH—C(O)—. In embodiments, L¹ is —NH—C(O)—(CH₂)—. In embodiments, L¹ is —NH—C(O)—(CH₂)₂—. In embodiments, L¹ is —NH—C(O)—(CH₂)₃—. In embodiments, L¹ is —NH—C(O)—O—(CH₂)_{y}—, and y is an integer from 0 to 3. In embodiments, L¹ is —NH—C(O)—O—. In embodiments, L¹ is —NH—C(O)—O—(CH₂)—. In embodiments, L¹ is —NH—C(O)—O—(CH₂)₂—. In embodiments, L¹ is —NH—C(O)—O—(CH₂)₃—.

With reference to the compounds described herein, L² is a bond, —NR^{2A}—, —S—, —S(O)₂—, —O—, —C(O)—, —C(O)O—, —OC(O)—, —N(R^{2A})C(O)—, —C(O)N(R^{2A})—, —NR^{2A}C(O)NR^{2B}—, —NR^{2A}C(NH)NR^{2B}—, —SO₂N(R^{2A})—, —N(R^{2A})SO₂—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L² is a bond, —NH—, —S—, —S(O)₂—, —O—, —C(O)—, —C(O)O—, —OC(O)—, —NHC(O)—, —C(O)NH—, —NHC(O)NH—, —NHC(NH)NH—, —SO₂NH—, —NHSO₂—, —C(S)—, L¹²-substituted or unsubstituted alkylene, L¹²-substituted or unsubstituted heteroalkylene, L¹²-substituted or unsubstituted cycloalkylene, L¹²-substituted or unsubstituted heterocycloalkylene, L¹²-substituted or unsubstituted arylene, or L¹²-substituted or unsubstituted heteroarylene. In embodiments, L² is a bond, —NH—, —S—, —S(O)₂—, —O—, —C(O)—, —C(O)O—, —OC(O)—, —NHC(O)—, —C(O)NH—, —NHC(O)NH—, —NHC(NH)NH—, —SO₂NH—, —NHSO₂—, —C(S)—, unsubstituted alkylene, unsubstituted heteroalkylene, unsubstituted cycloalkylene, unsubstituted heterocycloalkylene, unsubstituted arylene, or unsubstituted heteroarylene. In embodiments. L² is a bond. In embodiments, the alkylene is a C₁₋₆ alkylene. In embodiments, the alkylene is a C₁₋₄ alkylene. In embodiments, the heteroalkylene is a 2 to 6 membered heteroalkylene. In embodiments, the heteroalkylene is a 2 to 4 membered heteroalkylene. In embodiments, the cycloalkylene is a C₅-C₆ cycloalkylene. In embodiments, the heterocycloalkylene is a 5 or 6 membered heterocycloalkylene. In embodiments, the arylene is a C₅₋₆ arylene. In embodiments, the heteroarylene is a 5 or 6 membered heteroarylene.

With reference to the compounds described herein. R^{2A} and R^{2B} are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, the alkylene is a C₁₋₄ alkylene. In embodiments, the heteroalkylene is a 2 to 6 membered heteroalkylene. In embodiments, the heteroalkylene is a 2 to 4 membered heteroalkylene. In embodiments, the cycloalkylene is a C₅-C₆ cycloalkylene. In embodiments, the heterocycloalkylene is a 5 or 6 membered heterocycloalkylene. In embodiments, the arylene is a C₅₋₆ arylene. In embodiments, the heteroarylene is a 5 or 6 membered heteroarylene.

With reference to the compounds described herein, L¹² is halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —N(O)₂, —NHSO₂H, —NHC(O)H, —NHC(O)OH, —NHOH, —N₃, unsubstituted alkyl, unsubstituted heteroalkyl, unsubstituted cycloalkyl, unsubstituted heterocycloalkyl, unsubstituted aryl, or unsubstituted heteroaryl. In embodiments, the alkylene is a C₁₋₄ alkylene. In embodiments, the heteroalkylene is a 2 to 6 membered heteroalkylene. In embodiments, the heteroalkylene is a 2 to 4 membered heteroalkylene. In embodiments, the cycloalkylene is a C₅-C₆ cycloalkylene. In embodiments, the heterocycloalkylene is a 5 or 6 membered heterocycloalkylene. In embodiments, the arylene is a C₅₋₆ arylene. In embodiments, the heteroarylene is a 5 or 6 membered heteroarylene.

With reference to the compounds described herein, L³ is a bond, —N(R^{3A})—, —S—, —S(O)₂—, —O—, —C(O)—, —C(O)O—, —OC(O)—, —N(R^{3A})C(O)—, —C(O)N(R^{3A})—, —NR^{3A}C(O)NR^{3B}—, —NR^{3A}C(NH)NR^{3B}—, —SO₂N(R^{3A})—, —N(R^{3A})SO₂—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L³ is a bond, —NH—, —S—, —S(O)₂—, —O—, —C(O)—, —C(O)O—, —OC(O)—, —NHC(O)—, —C(O)NH—, —NHC(O)NH—, —NHC(NH)NH—, —SO₂NH—, —NHSO₂—, —C(S)—, L¹³-substituted or unsubstituted alkylene, L¹³-substituted or unsubstituted heteroalkylene, L¹³-substituted or unsubstituted cycloalkylene, L¹³-substituted or unsubstituted heterocycloalkylene, L¹³-substituted or unsubstituted arylene, or L¹³-substituted or unsubstituted heteroarylene. In embodiments, the alkylene is a C₁₋₄ alkylene. In embodiments, the heteroalkylene is a 2 to 6 membered heteroalkylene. In embodiments, the heteroalkylene is a 2 to 4 membered heteroalkylene. In embodiments, the cycloalkylene is a C₅-C₆ cycloalkylene. In embodiments, the heterocycloalkylene is a 5 or 6 membered heterocycloalkylene. In embodiments, the arylene is a C₅₋₆ arylene. In embodiments, the heteroarylene is a 5 or 6 membered heteroarylene.

With reference to the compounds described herein, R^{3A} and R^{3B} are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, the alkylene is a C₁₋₄ alkylene. In embodiments, the heteroalkylene is a 2 to 6 membered heteroalkylene. In embodiments, the heteroalkylene is a 2 to 4 membered heteroalkylene. In embodiments, the cycloalkylene is a C₅-C₆ cycloalkylene. In embodiments, the heterocycloalkylene is a 5 or 6 membered heterocycloalkylene. In embodiments, the arylene is a C₅₋₆ arylene. In embodiments, the heteroarylene is a 5 or 6 membered heteroarylene.

With reference to the compounds described herein, L¹³ is halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —N(O)₂, —NHSO₂H, —NHC(O)H, —NHC(O)OH, —NHOH, —N₃, unsubstituted alkyl, unsubstituted heteroalkyl, unsubstituted cycloalkyl, unsubstituted heterocycloalkyl, unsubstituted aryl, or unsubstituted heteroaryl. In embodiments, the alkylene is a C₁₋₄ alkylene, In embodiments, the heteroalkylene is a 2 to 6 membered heteroalkylene. In embodiments, the heteroalkylene is a 2 to 4 membered heteroalkylene. In embodiments, the cycloalkylene is a C₅-C₆ cycloalkylene. In embodiments, the heterocycloalkylene is a 5 or 6 membered heterocycloalkylene. In embodiments, the arylene is a C₅₋₆ arylene. In embodiments, the heteroarylene is a 5 or 6 membered heteroarylene.

With reference to the compounds described herein, L⁴ is a bond, —N(R^{4A})—, —S—, —S(O)₂—, —C(O)—, —C(O)O—, —O—, —OC(O)—, —N(R^{4A})C(O)—, —C(O)N(R^{4A})—, —NR^{4A}C(O)NR^{4B}, —NR^{4A}C(NH)NR^{4B}—, —SO₂N(R^{4A})—, —N(R^{4A})SO₂—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L⁴ is a bond, substituted or unsubstituted alkyl, or substituted or unsubstituted heteroalkyl. In embodiments, wherein L⁴ is a bond, unsubstituted alkyl, or unsubstituted heteroalkyl. In embodiments, wherein L⁴ is substituted alkyl. In embodiments, wherein L⁴ is substituted C₁₋₈ alkyl. In embodiments, wherein L⁴ is substituted C₁₋₆ alkyl. In embodiments, wherein L⁴ is substituted C₁₋₄ alkyl. In embodiments, wherein L⁴ is unsubstituted alkyl. In embodiments, wherein L⁴ is unsubstituted C₁₋₈ alkyl. In embodiments, wherein L⁴ is unsubstituted C₁₋₆ alkyl. In embodiments, wherein L⁴ is unsubstituted C₁₋₄ alkyl. In embodiments, wherein L⁴ is unsubstituted heteroalkyl. In embodiments, wherein L⁴ is unsubstituted 2 to 8 membered heteroalkyl. In embodiments, wherein L⁴ is unsubstituted 2 to 6 membered heteroalkyl. In embodiments, wherein L⁴ is unsubstituted 2 to 4 membered heteroalkyl. In embodiments, wherein L⁴ is substituted heteroalkyl. In embodiments, wherein L⁴ is substituted 2 to 8 membered heteroalkyl. In embodiments, wherein L⁴ is substituted 2 to 6 membered heteroalkyl. In embodiments, wherein L⁴ is substituted 2 to 4 membered heteroalkyl. In embodiments, L⁴ is a bond or —NH—(CH₂)_{y}—C(═O)—. In embodiments, L⁴ is a bond. In embodiments, L⁴ is —NH—(CH₂)—C(═O)—. In embodiments, L⁴ is —NH—(CH₂)₂—C(═O)—. In embodiments, L⁴ is —NH—(CH₂)_{y}—C(═O)—. In embodiments, L⁴ is —NH—(CH₂)₄—C(═O)—. In embodiments, L⁴ is —NH—(CH₂)₅—C(═O)—. In embodiments, L⁴ is —NH—(CH₂)₆—C(—O)—. In embodiments, L⁴ is —NH—(CH₂)₇—C(═O)—. In embodiments, L⁴ is —NH—(CH₂)₈—C(═O)—.

With reference to the compounds described herein, R^{4A} and R^{4B} are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R^{4A} and R^{4B} are independently hydrogen, substituted or unsubstituted C₁₋₄ alkyl, substituted or unsubstituted 2 to 4 membered heteroalkyl, substituted or unsubstituted C₅₋₆ cycloalkyl, substituted or unsubstituted 5 or 6 membered heterocycloalkyl, substituted or unsubstituted C₅₋₆ aryl, or substituted or unsubstituted 5 or 6 membered heteroaryl. In embodiments, R^{4A} and R^{4B} are independently hydrogen, unsubstituted C₁₋₄ alkyl, unsubstituted 2 to 4 membered heteroalkyl, unsubstituted C₅₋₆ cycloalkyl, unsubstituted 5 or 6 membered heterocycloalkyl, unsubstituted C₅₋₆ aryl, or unsubstituted 5 or 6 membered heteroaryl. In embodiments, R^{4A} and R^{4B} are independently hydrogen, substituted or unsubstituted C₁₋₄ alkyl, or substituted or unsubstituted 2 to 4 membered heteroalkyl. In embodiments, R^{4A} and R^{4B} are hydrogen. In embodiments, R^{4A} and R^{4B} are substituted or unsubstituted C₁₋₄ alkyl. In embodiments, R^{4A} and R^{4B} are unsubstituted C₁₋₄ alkyl. In embodiments, R^{4A} and R^{4B} are substituted or unsubstituted 2 to 4 membered heteroalkyl. In embodiments, R^{4A} and R^{4B} are unsubstituted 2 to 4 membered heteroalkyl.

With reference to the compounds described herein, L⁵ is a bond, —N(R^{5A})—, —S—, —S(O)₂—, —C(O)—, —C(O)O—, —O—, —OC(O)—, —N(R^{5A})C(O)—, —C(O)N(R^{5A})—, —NR^{5A}C(O)NR^{5B}, —NR^{5A}C(NH)NR^{5B}—, —SO₂N(R^{5A})—, —N(R^{5A})SO₂—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L⁵ is a bond, substituted or unsubstituted alkyl, or substituted or unsubstituted heteroalkyl. In embodiments, wherein L⁵ is a bond, unsubstituted alkyl, or unsubstituted heteroalkyl. In embodiments, wherein L⁵ is substituted alkyl. In embodiments, wherein L⁵ is substituted C₁₋₈ alkyl. In embodiments, wherein L⁵ is substituted C₁₋₆ alkyl. In embodiments, wherein L⁵ is substituted C₁₋₄ alkyl. In embodiments, wherein L⁴ is unsubstituted alkyl. In embodiments, wherein L⁵ is unsubstituted C₁₋₈ alkyl. In embodiments, wherein L⁵ is unsubstituted C₁₋₆ alkyl. In embodiments, wherein L⁵ is unsubstituted C₁₋₄ alkyl. In embodiments, wherein L⁵ is unsubstituted heteroalkyl. In embodiments, wherein L⁵ is unsubstituted 2 to 8 membered heteroalkyl. In embodiments, wherein L⁵ is unsubstituted 2 to 6 membered heteroalkyl. In embodiments, wherein L⁵ is unsubstituted 2 to 4 membered heteroalkyl. In embodiments, wherein L⁵ is substituted heteroalkyl. In embodiments, wherein L⁵ is substituted 2 to 8 membered heteroalkyl. In embodiments, wherein L⁵ is substituted 2 to 6 membered heteroalkyl. In embodiments, wherein L⁵ is substituted 2 to 4 membered heteroalkyl. In embodiments, L⁵ is a bond or —NH—(CH₂)_{y}—C(═O)—. In embodiments, L⁵ is a bond. In embodiments, L⁵ is —NH—(CH₂)—C(═O)—. In embodiments, L⁵ is —NH—(CH₂)₂—C(═O)—. In embodiments, L⁵ is —NH—(CH₂)₃—C(═O)—. In embodiments, L⁵ is —NH—(CH₂)₄—C(—O)—. In embodiments, L⁵ is —NH—(CH₂)₅—C(═O)—. In embodiments, L⁵ is —NH—(CH₂)₆—C(═O)—. In embodiments, L⁵ is —NH—(CH₂)₇—C(═O)—. In embodiments, L⁵ is —NH—(CH₂)₈—C(═O)—.

With reference to the compounds described herein, R^{5A} and R^{5B} are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R^{5A} and R^{5B} are independently hydrogen, substituted or unsubstituted C₁₋₄ alkyl, substituted or unsubstituted 2 to 4 membered heteroalkyl, substituted or unsubstituted C₅₋₆ cycloalkyl, substituted or unsubstituted 5 or 6 membered heterocycloalkyl, substituted or unsubstituted C₅₋₆ aryl, or substituted or unsubstituted 5 or 6 membered heteroaryl. In embodiments, R^{5A} and R^{5B} are independently hydrogen, unsubstituted C₁₋₄ alkyl, unsubstituted 2 to 4 membered heteroalkyl, unsubstituted C₅₋₆ cycloalkyl, unsubstituted 5 or 6 membered heterocycloalkyl, unsubstituted C₅₋₆ aryl, or unsubstituted 5 or 6 membered heteroaryl. In embodiments, R^{5A} and R^{5B} are independently hydrogen, substituted or unsubstituted C₁₋₄ alkyl, or substituted or unsubstituted 2 to 4 membered heteroalkyl. In embodiments, R^{5A} and R^{5B} are hydrogen. In embodiments, R^{5A} and R^{5B} are substituted or unsubstituted C₁₋₄ alkyl. In embodiments, R^{5A} and R^{5B} are unsubstituted C₁₋₄ alkyl. In embodiments, R^{5A} and R^{5B} are substituted or unsubstituted 2 to 4 membered heteroalkyl. In embodiments, R^{5A} and R^{5B} are unsubstituted 2 to 4 membered heteroalkyl.

With reference to the compounds described herein, R² is a first biomolecule moiety. In embodiments, R² is a peptidyl moiety, a lipid moiety, an RNA moiety, or a glycan moiety. In embodiments, R² is a lipid moiety. In embodiments, R² is a glycan moiety. In embodiments, R² is an RNA moiety. In embodiments, R² is a peptidyl moiety. In embodiments, the peptidyl moiety comprises a RNA-binding peptidyl moiety. In embodiments, the peptidyl moiety comprises a N⁶-methyladenosine reader peptidyl moiety. In embodiments, the peptidyl moiety comprises a N⁶-methyladenosine demethylase peptidyl moiety. In embodiments, the peptidyl moiety comprises a glycan-binding peptidyl moiety. In embodiments, the peptidyl moiety comprises Siglec-1. Siglec-2. Siglec-3, Siglec-4, Siglec-5, Siglec-6, Siglec-7, Siglec-8, Siglec-9, Siglec-10, Siglec-11, Siglec-12, Siglec-14, or Siglec-15. In embodiments, the protein moiety comprises Siglec-7. In embodiments, the peptidyl moiety comprises Siglec-7 (e.g., SEQ ID NO: 1, including embodiments as described herein). In embodiments, the peptidyl moiety comprises a sialoglycan binding V-set domain of Siglec. In embodiments, the peptidyl moiety comprises a sialoglycan binding V-set domain of Siglec-1, Siglec-2. Siglec-3, Siglec-4, Siglec-5. Siglec-6, Siglec-7, Siglec-8, Siglec-9, Siglec-10, Siglec-11, Siglec-12. Siglec-14, or Siglec-15. In embodiments, the peptidyl moiety comprises a sialoglycan binding V-set domain of Siglec-7 (e.g., SEQ ID NO:2, SEQ ID NO:3, or SEQ ID NO:4, including embodiments as described herein).

In embodiments, R² or the protein comprising an unnatural amino acid comprises a glycan-binding protein. In embodiments, R² or the protein comprising an unnatural amino acid comprises Siglec. In embodiments, R² or the protein comprising an unnatural amino acid comprises Siglec-1, Siglec-2, Siglec-3, Siglec-4, Siglec-5, Siglec-6, Siglec-7, Siglec-8, Siglec-9, Siglec-10, Siglec-11, Siglec-12, Siglec-14, or Siglec-15 In embodiments, R² or the protein comprising an unnatural amino acid comprises Siglec-1. In embodiments, R² or the protein comprising an unnatural amino acid comprises Siglec-2. In embodiments, R² or the protein comprising an unnatural amino acid comprises Siglec-3. In embodiments, R² or the protein comprising an unnatural amino acid comprises Siglec-4. In embodiments, R² or the protein comprising an unnatural amino acid comprises Siglec-5. In embodiments, R² or the protein comprising an unnatural amino acid comprises Siglec-6. In embodiments, R² or the protein comprising an unnatural amino acid comprises Siglec-8. In embodiments, R² or the protein comprising an unnatural amino acid comprises Siglec-9. In embodiments, R² or the protein comprising an unnatural amino acid comprises Siglec-10. In embodiments, R² or the protein comprising an unnatural amino acid comprises Siglec-11. In embodiments, R² or the protein comprising an unnatural amino acid comprises Siglec-12. In embodiments, R² or the protein comprising an unnatural amino acid comprises Siglec-14. In embodiments, R² or the protein comprising an unnatural amino acid comprises Siglec-15.

In embodiments, R² or the protein comprising an unnatural amino acid comprises Siglec-7. In embodiments, Siglec-7 comprises SEQ ID NO:1. In embodiments, Siglec-7 is SEQ ID NO:1. In embodiments, R² or the protein comprising the unnatural amino acid has at least 85% sequence identity to SEQ ID NO: 1. In embodiments, R² or the protein comprising the unnatural amino acid has at least 90% sequence identity to SEQ ID NO: 1. In embodiments, R² or the protein comprising the unnatural amino acid has at least 92% sequence identity to SEQ ID NO:1. In embodiments, R² or the protein comprising the unnatural amino acid has at least 94% sequence identity to SEQ ID NO: 1. In embodiments, R² or the protein comprising the unnatural amino acid has at least 95% sequence identity to SEQ ID NO: 1. In embodiments, R² or the protein comprising the unnatural amino acid has at least 96% sequence identity to SEQ ID NO:1. In embodiments, R² or the protein comprising the unnatural amino acid has at least 98% sequence identity to SEQ ID NO:1. In embodiments, the unnatural amino acid is at a lysine residue or asparagine residue in Siglec-7. In embodiments, the lysine residue is at position 104 or position 127 in SEQ ID NO: 1. In embodiments, the lysine residue is at position 104 in SEQ ID NO:1. In embodiments, the lysine residue is at position 127 in SEQ ID NO:1. In embodiments, the asparagine residue is at position 129 in SEQ ID NO: 1.

In embodiments, R² or the protein comprising an unnatural amino acid comprises the glycan binding domain of a glycan-binding protein. In embodiments, R² or the protein comprising an unnatural amino acid comprises the sialoglycan binding V-set domain of Siglec. In embodiments, R² or the protein comprising an unnatural amino acid comprises the sialoglycan binding V-set domain of Siglec-1, the sialoglycan binding V-set domain of Siglec-2, the sialoglycan binding V-set domain of Siglec-3, the sialoglycan binding V-set domain of Siglec-4, the sialoglycan binding V-set domain of Siglec-5, the sialoglycan binding V-set domain of Siglec-6, the sialoglycan binding V-set domain of Siglec-7, the sialoglycan binding V-set domain of Siglec-8, the sialoglycan binding V-set domain of Siglec-9, the sialoglycan binding V-set domain of Siglec-10, the sialoglycan binding V-set domain of Siglec-11, the sialoglycan binding V-set domain of Siglec-12, the sialoglycan binding V-set domain of Siglec-14, or the sialoglycan binding V-set domain of Siglec-15. In embodiments, R² or the protein comprising an unnatural amino acid comprises the sialoglycan binding V-set domain of Siglec-1. In embodiments, R² or the protein comprising an unnatural amino acid comprises the sialoglycan binding V-set domain of Siglec-2. In embodiments, R² or the protein comprising an unnatural amino acid comprises the sialoglycan binding V-set domain of Siglec-3. In embodiments. R² or the protein comprising an unnatural amino acid comprises the sialoglycan binding V-set domain of Siglec-4. In embodiments, R² or the protein comprising an unnatural amino acid comprises the sialoglycan binding V-set domain of Siglec-5. In embodiments, R² or the protein comprising an unnatural amino acid comprises the sialoglycan binding V-set domain of Siglec-6. In embodiments, R² or the protein comprising an unnatural amino acid comprises the sialoglycan binding V-set domain of Siglec-8. In embodiments, R² or the protein comprising an unnatural amino acid comprises the sialoglycan binding V-set domain of Siglec-9. In embodiments, R² or the protein comprising an unnatural amino acid comprises the sialoglycan binding V-set domain of Siglec-10. In embodiments, R² or the protein comprising an unnatural amino acid comprises the sialoglycan binding V-set domain of Siglec-11. In embodiments, R² or the protein comprising an unnatural amino acid comprises the sialoglycan binding V-set domain of Siglec-14. In embodiments, R² or the protein comprising an unnatural amino acid comprises the sialoglycan binding V-set domain of Siglec-14. In embodiments, R² or the protein comprising an unnatural amino acid comprises the sialoglycan binding V-set domain of Siglec-15.

In embodiments, R² or the protein comprising an unnatural amino acid comprises the sialoglycan binding V-set domain of Siglec-7. In embodiments, the sialoglycan binding V-set domain of Siglec-7 comprises SEQ ID NO:2. In embodiments, the sialoglycan binding V-set domain of Siglec-7 is SEQ ID NO:2. In embodiments, the sialoglycan binding V-set domain of Siglec-7 has at least 85% sequence identity to the amino acid sequence of SEQ ID NO:2. In embodiments, the sialoglycan binding V-set domain of Siglec-7 has at least 90% sequence identity to the amino acid sequence of SEQ ID NO:2. In embodiments, the sialoglycan binding V-set domain of Siglec-7 has at least 92% sequence identity to the amino acid sequence of SEQ ID NO:2. In embodiments, the sialoglycan binding V-set domain of Siglec-7 has at least 94% sequence identity to the amino acid sequence of SEQ ID NO:2. In embodiments, the sialoglycan binding V-set domain of Siglec-7 has at least 95% sequence identity to the amino acid sequence of SEQ ID NO:2. In embodiments, the sialoglycan binding V-set domain of Siglec-7 has at least 96% sequence identity to the amino acid sequence of SEQ ID NO:2. In embodiments, the sialoglycan binding V-set domain of Siglec-7 has at least 98% sequence identity to the amino acid sequence of SEQ ID NO:2. In embodiments, the unnatural amino acid is at a lysine residue or asparagine residue in the sialoglycan binding V-set domain of Siglec-7. In embodiments, the lysine residue is at position 104 or position 127 in SEQ ID NO:2. In embodiments, the lysine residue is at position 104 in SEQ ID NO:2. In embodiments, the lysine residue is at position 127 in SEQ ID NO:2. In embodiments, the asparagine residue is at position 129 in SEQ ID NO:2.

In embodiments, the sialoglycan binding V-set domain of Siglec-7 comprises SEQ ID NO:3. In embodiments, the sialoglycan binding V-set domain of Siglec-7 is SEQ ID NO:3. In embodiments, the sialoglycan binding V-set domain of Siglec-7 has at least 85% sequence identity to the amino acid sequence of SEQ ID NO:3. In embodiments, the sialoglycan binding V-set domain of Siglec-7 has at least 90% sequence identity to the amino acid sequence of SEQ ID NO:3. In embodiments, the sialoglycan binding V-set domain of Siglec-7 has at least 92% sequence identity to the amino acid sequence of SEQ ID NO:3. In embodiments, the sialoglycan binding V-set domain of Siglec-7 has at least 94% sequence identity to the amino acid sequence of SEQ ID NO:3. In embodiments, the sialoglycan binding V-set domain of Siglec-7 has at least 95% sequence identity to the amino acid sequence of SEQ ID NO:3. In embodiments, the sialoglycan binding V-set domain of Siglec-7 has at least 96% sequence identity to the amino acid sequence of SEQ ID NO:3. In embodiments, the sialoglycan binding V-set domain of Siglec-7 has at least 98% sequence identity to the amino acid sequence of SEQ ID NO:3. In embodiments, the unnatural amino acid is at a lysine residue or asparagine residue in the sialoglycan binding V-set domain of Siglec-7. In embodiments, the lysine residue is at position 104 or position 127 in SEQ ID NO:3. In embodiments, the lysine residue is at position 104 in SEQ ID NO:3. In embodiments, the lysine residue is at position 127 in SEQ ID NO:3. In embodiments, the asparagine residue is at position 129 in SEQ ID NO:3.

In embodiments, the sialoglycan binding V-set domain of Siglec-7 comprises SEQ ID NO:4. In embodiments, the sialoglycan binding V-set domain of Siglec-7 is SEQ ID NO:4. In embodiments, the sialoglycan binding V-set domain of Siglec-7 has at least 85% sequence identity to the amino acid sequence of SEQ ID NO:4. In embodiments, the sialoglycan binding V-set domain of Siglec-7 has at least 90% sequence identity to the amino acid sequence of SEQ ID NO:4. In embodiments, the sialoglycan binding V-set domain of Siglec-7 has at least 92% sequence identity to the amino acid sequence of SEQ ID NO:4. In embodiments, the sialoglycan binding V-set domain of Siglec-7 has at least 94% sequence identity to the amino acid sequence of SEQ ID NO:4. In embodiments, the sialoglycan binding V-set domain of Siglec-7 has at least 95% sequence identity to the amino acid sequence of SEQ ID NO:4. In embodiments, the sialoglycan binding V-set domain of Siglec-7 has at least 96% sequence identity to the amino acid sequence of SEQ ID NO:4. In embodiments, the sialoglycan binding V-set domain of Siglec-7 has at least 98% sequence identity to the amino acid sequence of SEQ ID NO:4. In embodiments, the unnatural amino acid is at a lysine residue or asparagine residue in the sialoglycan binding V-set domain of Siglec-7. In embodiments, the lysine residue is at position 104 or position 127 in SEQ ID NO:4. In embodiments, the lysine residue is at position 104 in SEQ ID NO:4. In embodiments, the lysine residue is at position 127 in SEQ ID NO:4. In embodiments, an asparagine residue in the sialoglycan binding V-set domain of Siglec-7 comprises an unnatural amino acid side chain of Formula (II), including embodiments thereof. In embodiments, the asparagine residue is at position 129 in SEQ ID NO:4.

With reference to the compounds described herein. R³ is a second biomolecule moiety. In embodiments, R³ is a peptidyl moiety, a lipid moiety. RNA, or a glycan moiety. In embodiments. R³ is a peptidyl moiety. In embodiments, R³ is a lipid moiety. In embodiments, R³ is a RNA moiety. In embodiments, L³ is bonded to a hydroxyl group within the RNA moiety. In embodiments, L³ is bonded to 2′-hydroxyl group within the RNA moiety. In embodiments, L³ is bonded to 2′-hydroxyl group of a ribose or an amine within the RNA moiety. In embodiments, L³ is bonded to 2′-hydroxyl group of a ribose within the RNA moiety. In embodiments, L³ is bonded to 2′-hydroxyl group of an amine within the RNA moiety. In embodiments, L³ is a bond. By targeting the 2′-hydroxyl group of a ribose, the RNA-binding protein can crosslink with all four nucleotides.

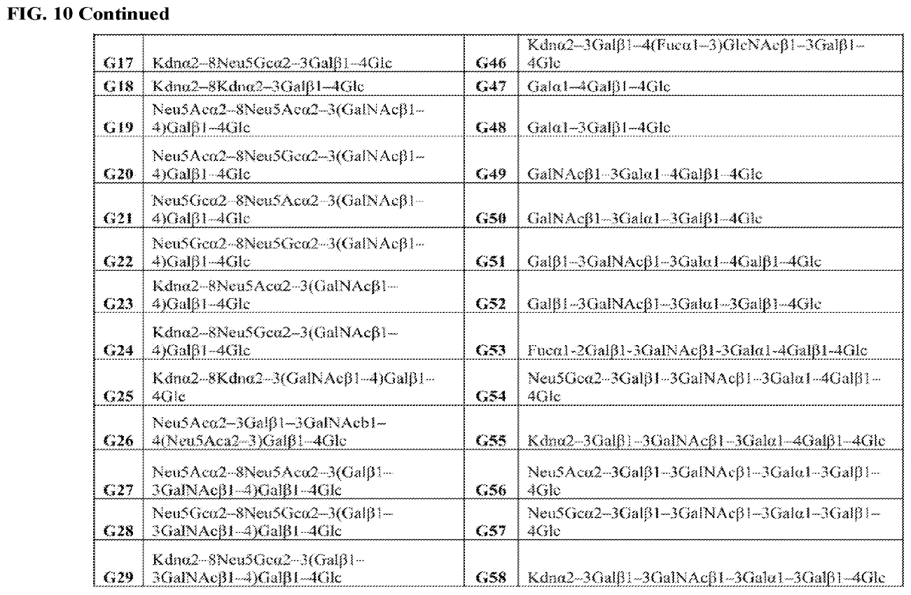

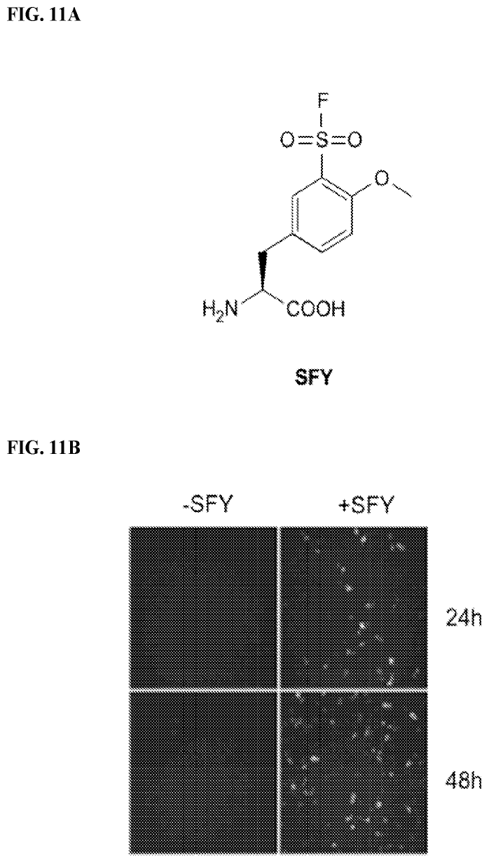

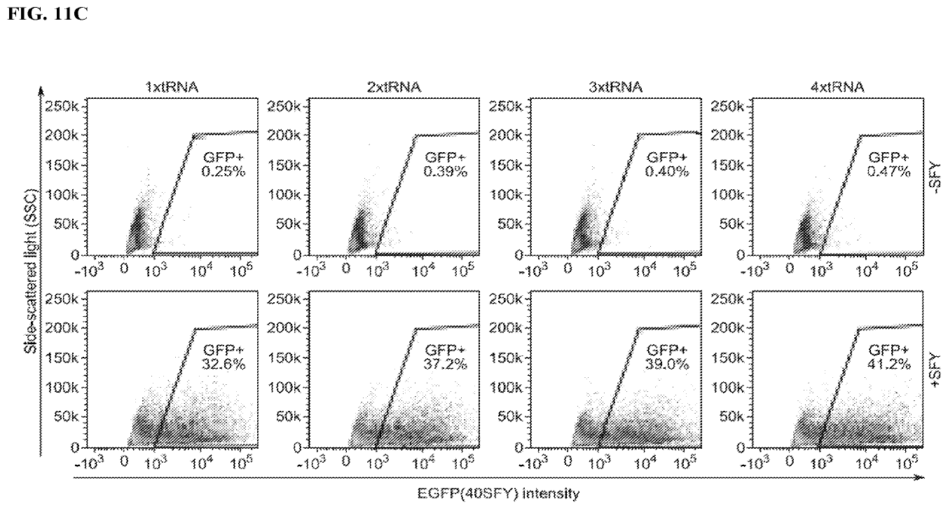

In embodiments of the compounds described herein. R³ is a glycan moiety. In embodiments, R³ is a sialoglycan moiety. In embodiments, a hydroxyl group of the glycan moiety bonds to L³ via an oxygen atom (—O—) within the glycan moiety, represented as —O-L³-. In embodiments, a hydroxyl group of the sialoglycan moiety bonds to L³ via an oxygen atom (—O—) within the sialoglycan moiety, represented as —O-L³-. In embodiments, L³ is a bond, such that the oxygen atom that is part of the structure of the sialoglycan moiety is bonded to the sulfur atom of the unnatural amino acid side chain. In embodiments, L³ is bonded to a sialoglycan containing a terminal 2,8-linked sialic acid (i.e., the unnatural amino acid side chain binds to a 2,8-linked sialic acid in a glycan). In embodiments, L³ is a bond. In embodiments, L³ is bonded to a sialoglycan containing a linear Neu5 Acα2-8Neu5Ac-terminating ligand, e.g., Neu5Acα2-8Neu5Acα2-3Galβ1-4Glc, Neu5 Acα2-8Neu5Gcα2-3Galβ1-4Glc, Neu5Acα2-8Kdncα2-3Galβ1-4Glc, Neu5Gcα2-8Neu5Acα2-3Galβ1-4Glc, or Neu5Gcα2-8Neu5Gcα2-3Galβ1-4Glc, shown in FIG. 10 as G11-G15, respectively. In embodiments, L³ is a bond. In embodiments, L³ is bonded to a sialoglycan containing an asymmetrically branched Neu5Acα2-8Neu5Ac-terminating ligands (e.g., G19-G22 or G27-G31 in FIG. 10). In embodiments, L³ is a bond.

With reference to the compounds described herein, R² is a peptidyl moiety, a lipid moiety, an RNA moiety, or a glycan moiety; and R³ is a peptidyl moiety, a lipid moiety, an RNA moiety, or a glycan moiety. In embodiments, R² is a peptidyl moiety, a lipid moiety, or an RNA moiety; and R³ is a glycan moiety. In embodiments, R² is a peptidyl moiety and R³ is a glycan moiety. In embodiments, R² is a lipid moiety and R³ is a glycan moiety. In embodiments, R² is an RNA moiety and R³ is a glycan moiety. In embodiments, R² is a peptidyl moiety and R³ is a peptidyl moiety.

In embodiments of the biomolecule conjugates described herein, the compound of Formula (III) further comprises a protein, a lipid, or RNA bonded to R³. In embodiments, the compound of Formula (III) further comprises a protein, a lipid, or RNA bonded to R³. In embodiments, the compound of Formula (III) further comprises a protein bonded to R³. In embodiments, the compound of Formula (III) further comprises a lipid bonded to R³. In embodiments, the compound of Formula (III) further comprises RNA bonded to R³. In embodiments, the lipid comprises a lipid membrane of a cell. In embodiments, the lipid comprises a lipid membrane of a cancer cell. In embodiments, the bond is a direct bond. In embodiments, the bond is an indirect bond. In embodiments, the bond is an electrostatic interaction (e.g., ionic bond, hydrogen bond, halogen bond). In embodiments, the bond is a van der Waals interaction (e.g., dipole-dipole, dipole-induced dipole, London dispersion). In embodiments, the bond is ring stacking (pi effects). In embodiments, the bond is a hydrophobic interaction.

In embodiments, the compound of Formula (III) further comprising a protein, a lipid, or RNA bonded to R³ is represented by the compound of Formula (IIIC):

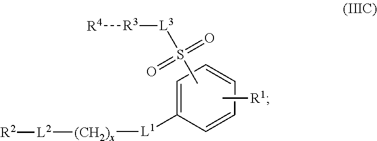

where x, L¹, L², L³, R², and R³ are as defined herein. The dashed line (-----) is a bond. R⁴ is a protein, a lipid, or RNA. In embodiments, R⁴ is a protein, a lipid, or RNA. In embodiments, R⁴ is a protein. In embodiments, R⁴ is a lipid. In embodiments, R⁴ is RNA. In embodiments, the lipid comprises a lipid membrane of a cell. In embodiments, the lipid comprises a lipid membrane of a cancer cell. In embodiments, the bond (-----) is a direct bond. In embodiments, the bond (-----) is an indirect bond. In embodiments, the bond is an electrostatic interaction (e.g., ionic bond, hydrogen bond, halogen bond). In embodiments, the bond is a van der Waals interaction (e.g., dipole-dipole, dipole-induced dipole, London dispersion). In embodiments, the bond is ring stacking (pi effects). In embodiments, the bond is a hydrophobic interaction.

### Cellular Compositions

The disclosure provides cells comprising the compounds, compositions and complexes provided herein, including embodiments thereof. Therefore, in an embodiment is provided a cell including the compound of Formula (I) and embodiments thereof, the compound of Formula (II) and embodiments thereof, the compound of Formula (III) and embodiments thereof, the compound of Formula (IV) and embodiments thereof, or the compound of Formula (V) and embodiments thereof.

In embodiments, the cell further includes a mutant pyrrolysyl-tRNA synthetase as described herein, including embodiments thereof. In embodiments, the cell further includes a vector as described herein, including embodiments thereof. In embodiments, the cell further includes a tRNA^{Pyl}.

In embodiments, the compound of Formula (I) (including embodiments thereof) is biosynthesized inside the cell, thereby generating a cell containing the compound of Formula (I). In embodiments, the compound of Formula (I) is contained in the medium outside the cell and penetrates into the cell, thereby generating a cell containing the compound of Formula (I). In embodiments, the cell comprises the compound of Formula (II) (including embodiments thereof). In embodiments, the cell comprises the compound of Formula (II) that is synthesized inside the cell. In embodiments, the cell comprises the compound of Formula (II) that is synthesized outside a cell, and that penetrates into the cell.

In embodiments, the cell comprises the biomolecule conjugates described herein. In embodiments, the cell comprises biomolecule conjugate of Formula (III), including embodiments thereof.

A cell can be any prokaryotic or eukaryotic cell. In aspects, the cell is prokaryotic. In aspects, the cell is eukaryotic. In aspects, the cell is a bacterial cell, a fungal cell, a plant cell, an archael cell, or an animal cell. In aspects, the animal cell is an insect cell or a mammalian cell. In aspects, the cell is a bacterial cell. In aspects, the cell is a fungal cell. In aspects, the cell is a plant cell. In aspects, the cell is an archael cell. In aspects, the cell is an animal cell. In aspects, the cell is an insect cell. In aspects, the cell is a mammalian cell. In aspects, the cell is a human cell. For example, any of the compositions described herein can be expressed in bacterial cells such as E. coli, insect cells, yeast or mammalian cells (such as Hela cells, Chinese hamster ovary cells (CHO) or COS cells). In aspects, the cell is a premature mammalian cell, i.e., a pluripotent stem cell. In aspects, the cell is derived from other human tissue. Other suitable cells are known to those skilled in the art.

### Pyrrolysyl-tRNA Synthetase

As described herein, an unnatural amino acid (e.g., of Formula (I) and embodiments thereof) may be inserted into or replace a naturally occurring amino acid in a biomolecule (e.g., protein). In order for the unnatural amino acid to be inserted or replace an amino acid in a biomolecule (e.g., protein), it must be capable of being incorporated during proteinogenesis. Thus, the unnatural amino acid must be present on a transfer RNA molecule (tRNA) such that it may be used in translation. Loading of amino acids occurs via an aminoacyl-tRNA synthetase, which is an enzyme that facilitates the attachment of appropriate amino acids to tRNA molecules. However, the attachment of unnatural amino acids to tRNA may not necessarily be accomplished by the naturally occurring aminoacyl-tRNA synthetase. Engineered aminoacyl-tRNA synthetases (e.g., mutant pyrrolysyl-tRNA synthetase (PyIRS)) may be useful for attaching unnatural amino acids to tRNA. A PyIRS mutant library was generated. Compared to previously described PyIRS mutant library, the PyIRS mutant library generated herein was constructed using the new small-intelligent mutagenesis approach that allows a greater number of amino acid residues to be mutated simultaneously (e.g., 10 amino acid residues).

The disclosure provides a mutant pyrrolysyl-tRNA synthetase, including at least 5 amino acid residues substitutions within the substrate-binding site of the mutant pyrrolysyl-tRNA synthetase. In embodiments, the mutant pyrrolysyl-tRNA synthetase is a mutant Methanosarcina mazei PylRS (e.g., SEQ ID NO:5). In embodiments, the mutant pyrrolysyl-tRNA synthetase comprises at least 5 amino acid residues substitutions in the amino acid sequence of SEQ ID NO:5. In embodiments, the substrate-binding site includes residues tyrosine at position 306, leucine at position 309, asparagine at position 346, cysteine at position 348, and tryptophan at position 417 as set forth in the amino acid sequence of SEQ ID NO:5. In embodiments, the at least 5 amino acid residues substitutions are leucine for tyrosine at position 306 (Y306L), alanine for leucine at position 309 (L309A), alanine for asparagine at position 346 (N346A), methionine for cysteine at position 348 (C348M), and threonine for tryptophan at position 417 (W417T) as set forth in the amino acid sequence of SEQ ID NO:5.

In embodiments, the mutant pyrrolysyl-tRNA synthetase has the amino acid sequence of SEQ ID NO:6. In embodiments, the mutant pyrrolysyl-tRNA synthetase includes an amino acid sequence of SEQ ID NO:6. In embodiments, the mutant pyrrolysyl-tRNA synthetase has an amino acid sequence that has at least 80%, 85%, 90%. 91%, 92%, 93%, 94%. 95%, 96%, 97%, 98%, 99%, or 100% identity to SEQ ID NO:6. In embodiments, the mutant pyrrolysyl-tRNA synthetase has an amino acid sequence that has at least 80% identity to SEQ ID NO:6. In embodiments, the mutant pyrrolysyl-tRNA synthetase has an amino acid sequence that has at least 85% identity to SEQ ID NO:6. In embodiments, the mutant pyrrolysyl-tRNA synthetase has an amino acid sequence that has at least 90% identity to SEQ ID NO:6. In embodiments, the mutant pyrrolysyl-tRNA synthetase has an amino acid sequence that has at least 92% identity to SEQ ID NO:6. In embodiments, the mutant pyrrolysyl-tRNA synthetase has an amino acid sequence that has at least 94% identity to SEQ ID NO:6. In embodiments, the mutant pyrrolysyl-tRNA synthetase has an amino acid sequence that has at least 95% identity to SEQ ID NO:6. In embodiments, the mutant pyrrolysyl-tRNA synthetase has an amino acid sequence that has at least 96% identity to SEQ ID NO:6. In embodiments, the mutant pyrrolysyl-tRNA synthetase has an amino acid sequence that has at least 98% identity to SEQ ID NO:6.

The disclosure provides a mutant pyrrolysyl-tRNA synthetase, including at least 5 amino acid residues substitutions within the substrate-binding site of the mutant pyrrolysyl-tRNA synthetase. In embodiments, the mutant pyrrolysyl-tRNA synthetase is a mutant Methanomethylophilus alvus PylRS (e.g., SEQ ID NO:7). In embodiments, the mutant pyrrolysyl-tRNA synthetase comprises at least 5 amino acid residues substitutions in the amino acid sequence of SEQ ID NO:7. In embodiments, the substrate-binding site includes residues tyrosine at position 126, leucine at position 309, methionine at position 129, asparagine at position 166, valine at position 168, and tryptophan at position 239 as set forth in the amino acid sequence of SEQ ID NO:7. In embodiments, the at least 5 amino acid residues substitutions are leucine for tyrosine at position 126 (Y126L), alanine for methionine at position 129 (M129A), alanine for asparagine at position 166 (N166A), methionine for valine at position 168 (V168M), and threonine for tryptophan at position 239 (W239T) as set forth in the amino acid sequence of SEQ ID NO:7.

In embodiments, the mutant pyrrolysyl-tRNA synthetase has the amino acid sequence of SEQ ID NO:8. In embodiments, the mutant pyrrolysyl-tRNA synthetase includes an amino acid sequence of SEQ ID NO:8. In embodiments, the mutant pyrrolysyl-tRNA synthetase has an amino acid sequence that has at least 80%, 85%, 90%, 91%, 92%, 93%, 94%, 95%, 96%, 97%, 98%, 99%, or 100% identity to SEQ ID NO:8. In embodiments, the mutant pyrrolysyl-tRNA synthetase has an amino acid sequence that has at least 80% identity to SEQ ID NO:8. In embodiments, the mutant pyrrolysyl-tRNA synthetase has an amino acid sequence that has at least 85% identity to SEQ ID NO:8. In embodiments, the mutant pyrrolysyl-tRNA synthetase has an amino acid sequence that has at least 90% identity to SEQ ID NO:8. In embodiments, the mutant pyrrolysyl-tRNA synthetase has an amino acid sequence that has at least 92% identity to SEQ ID NO:8. In embodiments, the mutant pyrrolysyl-tRNA synthetase has an amino acid sequence that has at least 94% identity to SEQ ID NO:8. In embodiments, the mutant pyrrolysyl-tRNA synthetase has an amino acid sequence that has at least 95% identity to SEQ ID NO:8. In embodiments, the mutant pyrrolysyl-tRNA synthetase has an amino acid sequence that has at least 96% identity to SEQ ID NO:8. In embodiments, the mutant pyrrolysyl-tRNA synthetase has an amino acid sequence that has at least 98% identity to SEQ ID NO:8.

### Vectors

The compositions (e.g., mutant pyrrolysyl-tRNA synthetase, tRNA^{Pyl}) provided herein may be delivered to cells using methods well known in the art. Thus, in an embodiment is provided a vector including a nucleic acid sequence encoding a mutant pyrrolysyl-tRNA synthetase as described herein, including embodiments thereof. In embodiments, the vector comprises a nucleic acid sequence encoding a mutant pyrrolysyl-tRNA synthetase that comprises at least 5 amino acid residues substitutions within the substrate-binding site of the mutant pyrrolysyl-tRNA synthetase. In embodiments, the vector further includes a nucleic acid sequence encoding tRNA^{Pyl}. In embodiments, the vector comprises a nucleic acid sequence encoding a mutant pyrrolysyl-tRNA synthetase as described herein. In embodiments, the vector further includes a nucleic acid sequence encoding tRNA^{Pyl}.

### Methods of Forming a Biomolecule or Biomolecule Conjugate

The compositions provided herein are useful for forming a biomolecule or biomolecule conjugate. Thus, in an embodiment is provided method of forming a biomolecule (e.g., protein) by contacting a biomolecule (e.g., protein such as Siglec-7 or a fragment thereof), a mutant pyrrolysyl-tRNA synthetase, a tRNA^{Pyl}, and a compound of Formula (I) (including embodiments thereof), thereby producing the biomolecule, i.e., a biomolecule comprising the unnatural amino acid of Formula (I) (including embodiments thereof). The biomolecule produced by the method will comprise the unnatural amino acid side chain of Formula (II) (including embodiments thereof). The mutant pyrrolysyl-tRNA synthetase used in the method of producing the biomolecule is any described herein. The tRNA^{Pyl} used in the method of producing the biomolecule is any described herein. In embodiments, the biomolecule is a protein. In embodiments, the biomolecule is a glycan. In embodiments, the reaction is performed in vitro. In embodiments, the reaction is performed in vivo. In embodiments, the reaction is performed in one or more living cells. In embodiments, the reaction is performed in one or more living bacterial cells. In embodiments, the reaction is performed in one or more living mammalian cells.

### Compositions

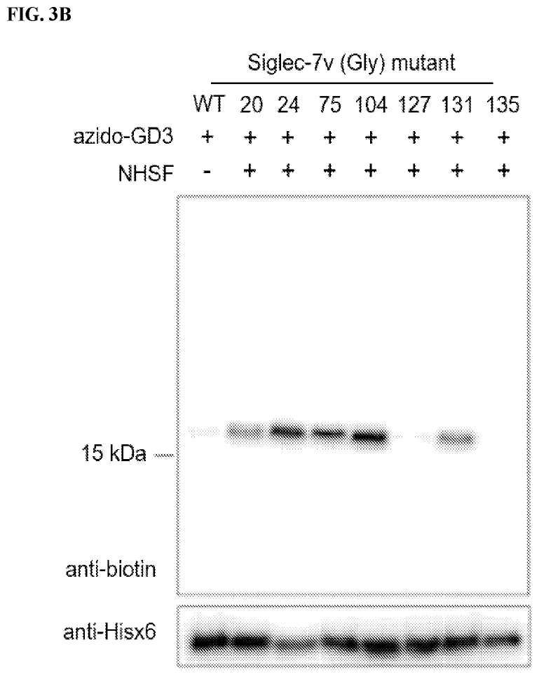

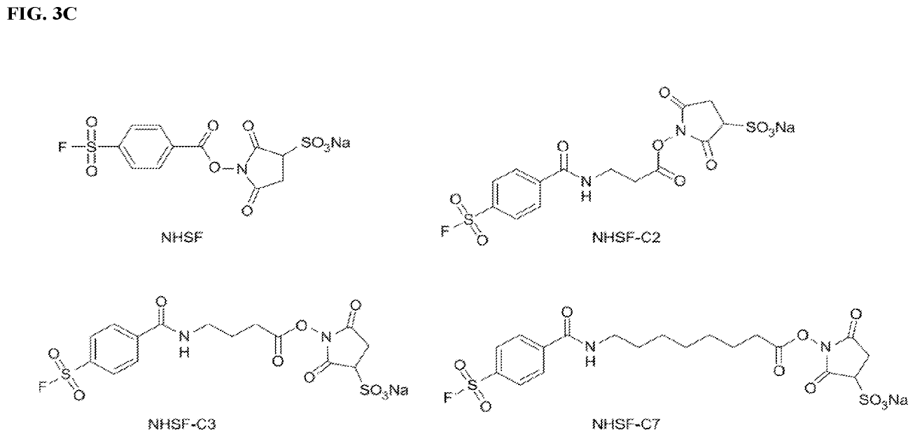

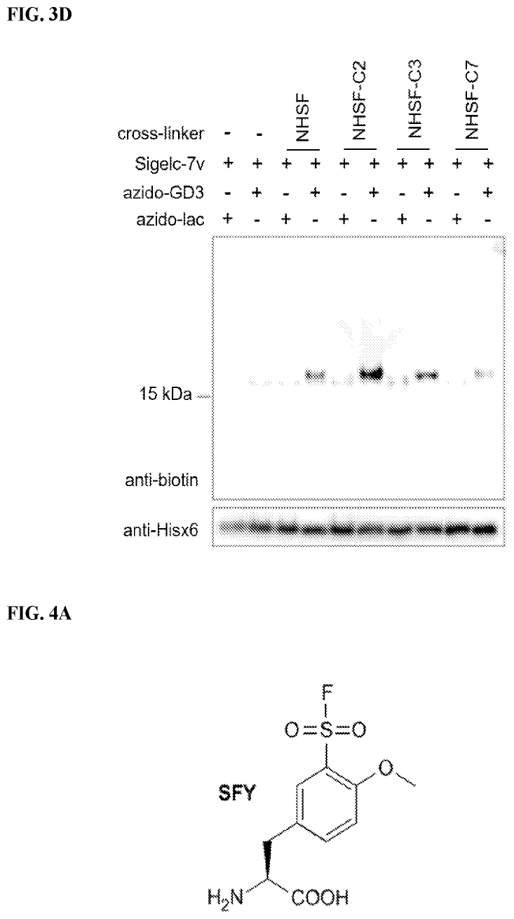

As shown in FIG. 3 and described in the examples, the compound of Formula (V) covalently targets specific protein-glycan interactions via proximity-enabled reactivity. When the protein, glycan, and compound of Formula (V) were placed in proximity, the compound of Formula (V) provided cross-linking between the protein and the glycan. Thus, the disclosure provides a composition comprising a protein, a glycan, and the compound of Formula (V), including embodiments thereof. The disclosure provides a composition comprising a protein and the compound of Formula (V), including embodiments thereof. The disclosure provides a composition comprising a glycan and the compound of Formula (V), including embodiments thereof. In embodiments, the disclosure provides a composition comprising Siglec-7 (e.g., SEQ ID NO: 1 and all embodiments thereof), a sialoglycan, and the compound of Formula (V) (and all embodiments thereof). In embodiments, the disclosure provides a composition comprising a sialoglycan binding V-set domain of Siglec-7 (e.g., SEQ ID NO:2 and all embodiments thereof), a sialoglycan, and the compound of Formula (V) (and all embodiments thereof). In embodiments, the disclosure provides a composition comprising a sialoglycan binding V-set domain of Siglec-7 (e.g., SEQ ID NO:3 and all embodiments thereof), a sialoglycan, and the compound of Formula (V) (and all embodiments thereof). In embodiments, the disclosure provides a composition comprising a sialoglycan binding V-set domain of Siglec-7 (e.g., SEQ ID NO:4 and all embodiments thereof), a sialoglycan, and the compound of Formula (V) (and all embodiments thereof). In embodiments, the disclosure provides a composition comprising a RNA-binding protein. RNA, and the compound of Formula (V) (and all embodiments thereof). In embodiments, the disclosure provides a composition comprising a N⁶-methyladenosine reader protein. RNA comprising N⁶-methyladenosine, and the compound of Formula (V) (and all embodiments thereof). In embodiments, the disclosure provides a composition comprising a N⁶-methyladenosine demethylase protein. RNA comprising N⁶-methyladenosine, and the compound of Formula (V) (and all embodiments thereof).

### Pharmaceutical Compositions

Provided herein are pharmaceutical compositions comprising: (i) a biomolecule which comprises an unnatural amino acid and (ii) a pharmaceutically acceptable excipient. In embodiments, the pharmaceutical compositions comprise (i) a lipid which comprises an unnatural amino acid and (ii) a pharmaceutically acceptable excipient. In embodiments, the pharmaceutical compositions comprise (i) RNA which comprises an unnatural amino acid and (ii) a pharmaceutically acceptable excipient. In embodiments, the pharmaceutical compositions comprise (i) a protein which comprises an unnatural amino acid and (ii) a pharmaceutically acceptable excipient. In embodiments, the pharmaceutical compositions comprise (i) a nucleic acid capable of encoding a protein which comprises an unnatural amino acid and (ii) a pharmaceutically acceptable excipient. In embodiments, the pharmaceutical compositions comprise (i) a vector which comprises a nucleic acid capable of encoding a protein which comprises an unnatural amino acid and (ii) a pharmaceutically acceptable excipient. In embodiments, the protein is a glycan binding protein or a fragment thereof. In embodiments, the protein is a sialoglycan binding protein or a fragment thereof. In embodiments, the pharmaceutical compositions comprise (i) the compound of Formula (II), wherein the protein comprises Siglec or a fragment thereof, and (ii) a pharmaceutically acceptable excipient. In embodiments, the pharmaceutical compositions comprise (i) the compound of Formula (II), wherein the protein comprises a sialoglycan binding V-set domain of Siglec or a fragment thereof, and (ii) a pharmaceutically acceptable excipient. In embodiments, the Siglec is Siglec-1, Siglec-2, Siglec-3, Siglec-4, Siglec-5, Siglec-6, Siglec-7, Siglec-8, Siglec-9, Siglec-10, Siglec-11, Siglec-12, Siglec-14, or Siglec-15. The compositions are suitable for formulation and administration in vitro or in vivo. Suitable carriers and excipients and their formulations are described in Remington: The Science and Practice of Pharmacy. 21st Edition. David B. Troy, ed., Lippicott Williams & Wilkins (2005).

In embodiments, the pharmaceutical compositions comprise (i) the compound of Formula (II), wherein the protein comprises Siglec-7 or a fragment thereof, and (ii) a pharmaceutically acceptable excipient. In embodiments, the pharmaceutical composition comprises (i) a nucleic acid encoding the compound of Formula (II), wherein the protein comprises Siglec-7 (or a fragment thereof), and (ii) a pharmaceutically acceptable excipient. In embodiments, the pharmaceutical composition comprises (i) a vector which comprises a nucleic acid encoding the compound of Formula (II), wherein the protein comprises Siglec-7 (or a fragment thereof), and (ii) a pharmaceutically acceptable excipient. In embodiments, the pharmaceutical compositions comprise (i) the compound of Formula (II), wherein the protein comprises SEQ ID NO: 1 (or any embodiment thereof), and (ii) a pharmaceutically acceptable excipient. In embodiments, the pharmaceutical composition comprises (i) a nucleic acid encoding the compound of Formula (II), wherein the protein comprises SEQ ID NO:1 (or any embodiment thereof), and (ii) a pharmaceutically acceptable excipient. In embodiments, the pharmaceutical composition comprises (i) a vector which comprises a nucleic acid encoding the compound of Formula (II), wherein the protein comprises SEQ ID NO: 1 (or any embodiment thereof), and (ii) a pharmaceutically acceptable excipient.

In embodiments, the pharmaceutical compositions comprise (i) the compound of Formula (II), wherein the protein comprises a sialoglycan binding V-set domain of Siglec-7, and (ii) a pharmaceutically acceptable excipient. In embodiments, the pharmaceutical composition comprises (i) a nucleic acid encoding the compound of Formula (II), wherein the protein comprises a sialoglycan binding V-set domain of Siglec-7, and (ii) a pharmaceutically acceptable excipient. In embodiments, the pharmaceutical composition comprises (i) a vector which comprises a nucleic acid encoding the compound of Formula (II), wherein the protein comprises a sialoglycan binding V-set domain of Siglec-7, and (ii) a pharmaceutically acceptable excipient. In embodiments, the pharmaceutical compositions comprise (i) the compound of Formula (II), wherein the protein comprises SEQ ID NO:2 (or any embodiment thereof), and (ii) a pharmaceutically acceptable excipient. In embodiments, the pharmaceutical composition comprises (i) a nucleic acid encoding the compound of Formula (II), wherein the protein comprises SEQ ID NO:2 (or any embodiment thereof), and (ii) a pharmaceutically acceptable excipient. In embodiments, the pharmaceutical composition comprises (i) a vector which comprises a nucleic acid encoding the compound of Formula (II), wherein the protein comprises SEQ ID NO:2 (or any embodiment thereof), and (ii) a pharmaceutically acceptable excipient.

In embodiments, the pharmaceutical compositions comprise (i) the compound of Formula (II), wherein the protein comprises SEQ ID NO:3 (or any embodiment thereof), and (ii) a pharmaceutically acceptable excipient. In embodiments, the pharmaceutical composition comprises (i) a nucleic acid encoding the compound of Formula (II), wherein the protein comprises SEQ ID NO:3 (or any embodiment thereof), and (ii) a pharmaceutically acceptable excipient. In embodiments, the pharmaceutical composition comprises (i) a vector which comprises a nucleic acid encoding the compound of Formula (II), wherein the protein comprises SEQ ID NO:3 (or any embodiment thereof), and (ii) a pharmaceutically acceptable excipient.

In embodiments, the pharmaceutical compositions comprise (i) the compound of Formula (II), wherein the protein comprises SEQ ID NO:4 (or any embodiment thereof), and (ii) a pharmaceutically acceptable excipient. In embodiments, the pharmaceutical composition comprises (i) a nucleic acid encoding the compound of Formula (II), wherein the protein comprises SEQ ID NO:4 (or any embodiment thereof), and (ii) a pharmaceutically acceptable excipient. In embodiments, the pharmaceutical composition comprises (i) a vector which comprises a nucleic acid encoding the compound of Formula (II), wherein the protein comprises SEQ ID NO:4 (or any embodiment thereof), and (ii) a pharmaceutically acceptable excipient.

In embodiments, the pharmaceutical composition comprises: (i) a RNA-binding protein comprising the compound of Formula (II) and (ii) a pharmaceutically acceptable excipient.

“Pharmaceutically acceptable excipient” and “pharmaceutically acceptable carrier” refer to a substance that aids the administration of an active agent to and absorption by a subject and can be included in the compositions of the disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful.

Solutions of the active compounds as free base or pharmacologically acceptable salt can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations can contain a preservative to prevent the growth of microorganisms.

Pharmaceutical compositions can be delivered via intranasal or inhalable solutions or sprays, aerosols or inhalants. Nasal solutions can be aqueous solutions designed to be administered to the nasal passages in drops or sprays. Nasal solutions can be prepared so that they are similar in many respects to nasal secretions. Thus, the aqueous nasal solutions usually are isotonic and slightly buffered to maintain a pH of 5 to 7. In addition, antimicrobial preservatives, similar to those used in ophthalmic preparations and appropriate drug stabilizers, if required, may be included in the formulation. Various commercial nasal preparations are known and can include, for example, antibiotics and antihistamines.

Oral formulations can include excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. In embodiments, oral pharmaceutical compositions will comprise an inert diluent or edible carrier, or they may be enclosed in hard or soft shell gelatin capsule, or they may be compressed into tablets, or they may be incorporated directly with the food. For oral therapeutic administration, the active compounds may be incorporated with excipients and used in the form of ingestible tablets, buccal tablets, troches, capsules, elixirs, suspensions, syrups, wafers, and the like. The percentage of the compositions and preparations may, of course, be varied and may conveniently be between about 1 to about 99% of the weight of the unit. The amount of active compounds in such compositions is such that a suitable dosage can be obtained.

For parenteral administration in an aqueous solution, for example, the solution should be suitably buffered and the liquid diluent first rendered isotonic with sufficient saline or glucose. Aqueous solutions, in particular, sterile aqueous media, are especially suitable for intravenous, intramuscular, subcutaneous and intraperitoneal administration. For example, one dosage could be dissolved in 1 ml of isotonic NaCl solution and either added to 1000 ml of hypodermoclysis fluid or injected at the proposed site of infusion.

Sterile injectable solutions can be prepared by incorporating the active compounds in the required amount in the appropriate solvent followed by filtered sterilization. Generally, dispersions are prepared by incorporating the various sterilized active ingredients into a sterile vehicle which contains the basic dispersion medium. Vacuum-drying and freeze-drying techniques, which yield a powder of the active ingredient plus any additional desired ingredients, can be used to prepare sterile powders for reconstitution of sterile injectable solutions. The preparation of more, or highly, concentrated solutions for direct injection is also contemplated. Organic solvents can be used for rapid penetration, delivering high concentrations of the active agents to a small area.

The formulations of compounds can be presented in unit-dose or multi-dose sealed containers, such as ampules and vials. Thus, the composition can be in unit dosage form. In such form the preparation is subdivided into unit doses containing appropriate quantities of the active component. Thus, the compositions can be administered in a variety of unit dosage forms depending upon the method of administration. For example, unit dosage forms suitable for oral administration include, but are not limited to, powder, tablets, pills, capsules and lozenges.

The dosage and frequency (single or multiple doses) of the pharmaceutical compositions comprising a protein which comprises an unnatural amino acid (e.g., a compound of Formula (II) and embodiments thereof) administered to a subject can vary depending upon a variety of factors, for example, whether the mammal suffers from another disease, and its route of administration; size, age, sex, health, body weight, body mass index, and diet of the recipient; nature and extent of symptoms of the disease being treated (e.g., symptoms of cancer and severity of such symptoms), kind of concurrent treatment, complications from the disease being treated or other health-related problems. Other therapeutic regimens or agents can be used in conjunction with the methods and compounds described herein. Adjustment and manipulation of established dosages (e.g., frequency and duration) are well within the ability of those skilled in the art.

For any composition and compound of Formula (V) (and embodiments thereof) described herein, the effective amount can be initially determined from cell culture assays. Target concentrations will be those concentrations that are capable of achieving the methods described herein, as measured using the methods described herein or known in the art. As is known in the art, effective amounts of the compounds and pharmaceutical compositions for use in humans can also be determined from animal models. For example, a dose for humans can be formulated to achieve a concentration that has been found to be effective in animals. The dosage in humans can be adjusted by monitoring effectiveness and adjusting the dosage upwards or downwards, as described above. Adjusting the dose to achieve maximal efficacy in humans based on the methods described above and other methods is well within the capabilities of the ordinarily skilled artisan.

Dosages of the compounds and pharmaceutical compositions may be varied depending upon the requirements of the patient. The dose administered to a patient should be sufficient to affect a beneficial therapeutic response in the patient over time. The size of the dose also will be determined by the existence, nature, and extent of any adverse side-effects. Determination of the proper dosage for a particular situation is within the skill of the art. Dosage amounts and intervals can be adjusted individually to provide levels of the compounds effective for the particular clinical indication being treated. This will provide a therapeutic regimen that is commensurate with the severity of the individual's disease state.

In embodiments, the compounds are administered to a patient at an amount of about 0.01 mg/kg to about 500 mg/kg. It is understood that where the amount is referred to as “mg/kg,” the amount is milligram per kilogram body weight of the subject being administered with the compounds described herein. In embodiments, the compound is administered to a patient in an amount from about 1 mg to about 500 mg per day, as a single dose, or in a dose administered two or three times per day.

### Methods

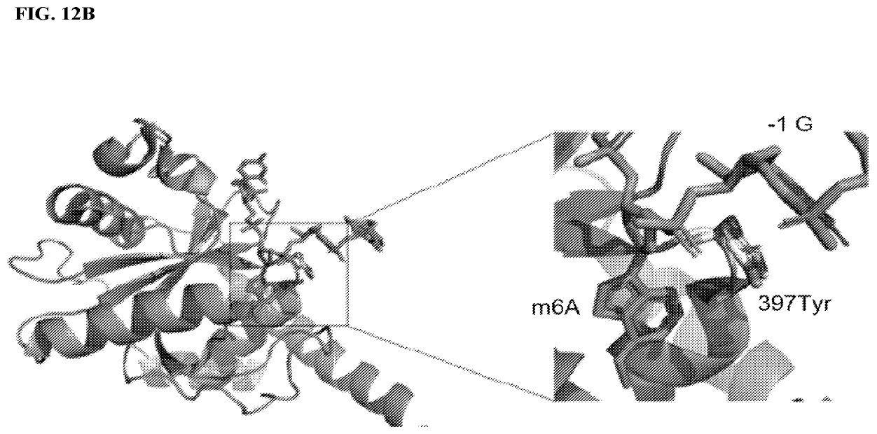

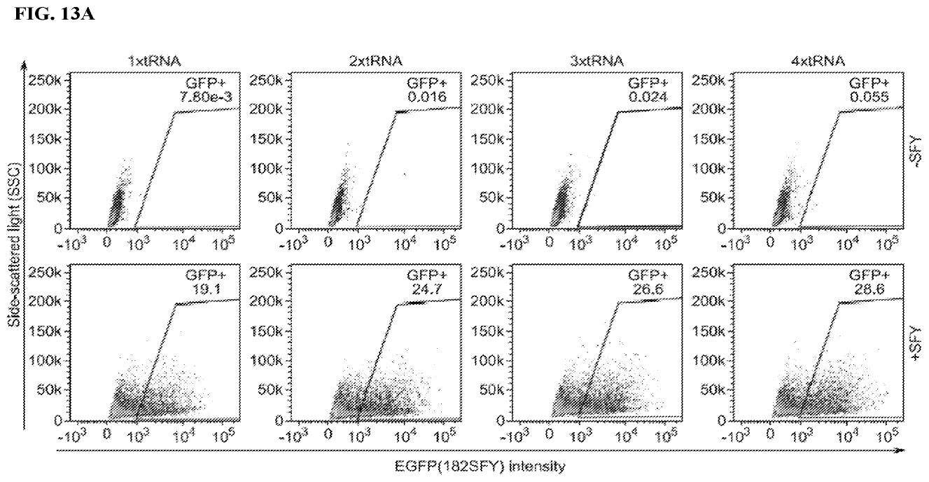

Provided herein are methods of identifying N⁶-methyladenosine (m⁶A) sites on RNA. e.g., by contacting an N⁶-methyladenosine reader protein which comprises the compound of Formula (I) (or an embodiment thereof) or Formula (II) (or an embodiment thereof) with RNA. Provided herein are in vivo methods of identifying N⁶-methyladenosine (m6A) sites on RNA in the transcriptome. Provided herein are in vivo methods of identifying N⁶-methyladenosine (m6A) sites on RNA in the transcriptome comprising incorporating the compound of Formula (I) (or an embodiment thereof) or Formula (II) (or an embodiment thereof) into the YTH domain in mammalian cells, and identifying N⁶-methyladenosine (m6A) sites through high-throughput sequencing. Provided herein are in vivo methods of identifying N⁶-methyladenosine (m6A) sites on RNA in the transcriptome comprising genetically incorporating the compound of Formula (I) (or an embodiment thereof) or Formula (II) (or an embodiment thereof) into the YTH domain in mammalian cells, and identifying N⁶-methyladenosine (m6A) sites through high-throughput sequencing. In embodiments, the method of identifying N⁶-methyladenosine (m6A) sites in RNA comprises contacting an N⁶-methyladenosine reader protein which comprises the compound of Formula (I) (or an embodiment thereof) or Formula (II) (or an embodiment thereof) with RNA. In embodiments, the N⁶-methyladenosine (m6A) sites in RNA are endogenous m6A sites in cells. In embodiments, the N⁶-methyladenosine (m⁶A) sites in RNA are endogenous m6A sites in mammalian cells. In embodiments, the RNA is in the transcriptome. In embodiments, the N⁶-methyladenosine (m6A) sites in RNA are endogenous m6A sites in the transcriptome in cells. In embodiments, the N⁶-methyladenosine (m6A) sites in RNA are endogenous m6A sites in the transcriptome in mammalian cells. The disclosure provides methods of detecting endogenous m6A sites in cells throughout the transcriptome comprising contacting an N⁶-methyladenosine reader protein which comprises the compound of Formula (I) (or an embodiment thereof) or Formula (II) (or an embodiment thereof) with RNA using high-throughput sequencing. In embodiments, the N⁶-methyladenosine reader protein comprises the compound of Formula (I) (or an embodiment thereof) or Formula (II) (or an embodiment thereof) at an N⁶-methyladenosine binding site of the N⁶-methyladenosine reader protein. Expression of the N⁶-methyladenosine reader protein comprises the compound of Formula (I) (or an embodiment thereof) or Formula (II) (or an embodiment thereof) crosslinks at N⁶-methyladenosine sites in RNA (FIG. 12A). Immunoprecipitation of the N⁶-methyladenosine reader protein followed with protease K digestion releases the captured RNAs for reverse transcription, adaptor ligation, and sequencing (FIG. 12A). In embodiments, the RNA is mRNA. The identified Formula (I)/Formula (II)-crosslinked nucleotides thus reveal the N⁶-methyladenosine site to be immediately adjacent. N⁶-methyladenosine reader proteins are known in the art and include Class I (e.g., proteins that contain a YTH domain). Class II (e.g., proteins that use an m⁶A-switch mechanism to bind m6A-containing transcripts, such as hnRNPC and hnRNPG), and Class III (e.g., proteins using a common RNA binding domain in a flanking region to recognize m6A-containing transcripts, such as IGF2BP). In embodiments, the N⁶-methyladenosine reader protein which comprises the compound of Formula (I) (or an embodiment thereof) or Formula (II) (or an embodiment thereof) is a YTH family protein (e.g., a protein that contains a YTH domain). In embodiments, the N⁶-methyladenosine reader protein is YTHDC1. YTHDC2. YTHDF1. YTHDF2, or YTHDF3. In embodiments, the N⁶-methyladenosine reader protein is YTHDC1. In embodiments, the N⁶-methyladenosine reader protein is YTHDC2. In embodiments, the N⁶-methyladenosine reader protein is YTHDF1. In embodiments, the N⁶-methyladenosine reader protein is YTHDF2. In embodiments, the N⁶-methyladenosine reader protein is YTHDF3. In embodiments, the N⁶-methyladenosine reader protein is hnRNPC or hnRNPG. In embodiments, the N⁶-methyladenosine reader protein is hnRNPC. In embodiments, the N⁶-methyladenosine reader protein is hnRNPG. In embodiments, the N⁶-methyladenosine reader protein is IGF2BP. In embodiments, the N⁶-methyladenosine reader protein is IGF2BP1. IGF2BP2, or IGF2PB3. In embodiments, the N⁶-methyladenosine reader protein is IGF2BP1. In embodiments, the N⁶-methyladenosine reader protein is IGF2BP2. In embodiments, the N⁶-methyladenosine reader protein is IGF2BP3. The method described herein provides an antibody-free approach for identifying m6A with single-nucleotide resolution in vivo, which will reflect m6A physiological status more closely. The methods described herein provide for high-throughput sequence mapping of all m6A in the transcriptome. In addition, the present methods can be generalized to map other RNA modifications in vivo for which a reader or binder exists.

Provided herein are methods of identifying N⁶-methyladenosine (m⁶A) sites on RNA. e.g., by contacting a N⁶-methyladenosine (m⁶A) demethylase (eraser) protein which comprises the compound of Formula (I) (or an embodiment thereof) or Formula (II) (or an embodiment thereof) with RNA. Provided herein are in vivo methods of identifying N⁶-methyladenosine (m⁶A) sites on RNA in the transcriptome. Provided herein are in vivo methods of identifying N⁶-methyladenosine (m⁶A) sites on RNA in the transcriptome comprising incorporating the compound of Formula (I) (or an embodiment thereof) or Formula (II) (or an embodiment thereof) into the YTH domain in mammalian cells, and identifying N⁶-methyladenosine (m⁶A) sites through high-throughput sequencing. Provided herein are in vivo methods of identifying N⁶-methyladenosine (m⁶A) sites on RNA in the transcriptome comprising genetically incorporating the compound of Formula (I) (or an embodiment thereof) or Formula (II) (or an embodiment thereof) into the YTH domain in mammalian cells, and identifying N⁶-methyladenosine (m⁶A) sites through high-throughput sequencing. In embodiments, the method of identifying N⁶-methyladenosine (m⁶A) sites in RNA comprises contacting a m⁶A demethylase protein which comprises the compound of Formula (I) (or an embodiment thereof) or Formula (II) (or an embodiment thereof) with RNA. In embodiments, the N⁶-methyladenosine (m⁶A) sites in RNA are endogenous m⁶A sites in cells. In embodiments, the N⁶-methyladenosine (m⁶A) sites in RNA are endogenous m⁶A sites in mammalian cells. In embodiments, the RNA is in the transcriptome. In embodiments, the N⁶-methyladenosine (m⁶A) sites in RNA are endogenous m⁶A sites in the transcriptome in cells. In embodiments, the N⁶-methyladenosine (m⁶A) sites in RNA are endogenous m⁶A sites in the transcriptome in mammalian cells. The disclosure provides methods of detecting endogenous m⁶A sites in cells throughout the transcriptome comprising contacting a m⁶A demethylase protein which comprises the compound of Formula (I) (or an embodiment thereof) or Formula (II) (or an embodiment thereof) with RNA using high-throughput sequencing. In embodiments, the m⁶A demethylase protein comprises the compound of Formula (I) (or an embodiment thereof) or Formula (II) (or an embodiment thereof) at an N⁶-methyladenosine binding site of the m⁶A demethylase protein. In embodiments, the m⁶A demethylase protein is FTO or ALKBH5. In embodiments, the m⁶A demethylase protein is FTO. In embodiments, the m⁶A demethylase protein is ALKBH5. The method described herein provides an antibody-free approach for identifying m⁶A with single-nucleotide resolution in vivo, which will reflect m⁶A physiological status more closely. The methods described herein provide for high-throughput sequence mapping of all m⁶A in the transcriptome. In addition, the present methods can be generalized to map other RNA modifications in vivo for which a reader or binder exists.

### Methods of Treatment

The disclosure provides methods of treating a disease in a patient in need thereof by administering to the patient an effective amount of the compounds or compositions described herein to treat the disease. In embodiments, the disease comprises an elevated level of sialoglycan relative to a control. In embodiments, the disclosure provides methods of treating cancer in a patient in need thereof by administering to the patient an effective amount of the compounds or compositions described herein to treat the cancer. In embodiments, the disclosure provides methods of treating cancer in a patient in need thereof by administering to the patient an effective amount of the compounds or compositions described herein to treat the cancer, wherein the cancer has an elevated level of sialoglycan relative to a control (e.g., an elevated level of sialoglycan on the cancer cells relative to a control). In embodiments, the disclosure provides methods of treating cancer in a patient in need thereof by administering to the patient an effective amount of the compounds or compositions described herein to treat the cancer, wherein the cancer comprises sialoglycan (e.g., sialoglycan on the cancer cells). In embodiments, the methods further comprise detecting an elevated level of sialoglycan in a biological sample obtained from the patient. In embodiments, the cancer is melanoma or breast cancer. In embodiments, the cancer is melanoma. In embodiments, the cancer is breast cancer. In embodiments, the breast cancer is breast carcinoma. In embodiments, the breast cancer is breast adenocarcinoma.

The disclosure provides methods of treating cancer in a patient in need thereof by detecting an elevated level of sialoglycan in a biological sample obtained from the patient, and administering to the patient an effective amount of the compounds or compositions described herein. In embodiments, the cancer is melanoma or breast cancer. In embodiments, the cancer is melanoma. In embodiments, the cancer is breast cancer. In embodiments, the breast cancer is breast carcinoma. In embodiments, the breast cancer is breast adenocarcinoma.

“Disease” or “condition” refer to a state of being or health status of a patient or subject capable of being treated with a compound, pharmaceutical composition, or method provided herein. The disease may be a cancer (e.g., ovarian cancer, bladder cancer, head and neck cancer, brain cancer, breast cancer, lung cancer, cervical cancer, liver cancer, colorectal cancer, pancreatic cancer, glioblastoma, neuroblastoma, rhabdomyosarcoma, osteosarcoma, renal cancer, renal cell carcinoma, non-small cell lung cancer, uterine cancer, testicular cancer, anal cancer, bile duct cancer, biliary tract cancer, gastrointestinal carcinoid tumors, esophageal cancer, gall bladder cancer, appendix cancer, small intestine cancer, stomach (gastric) cancer, urinary bladder cancer, genitourinary tract cancer, endometrial cancer, nasopharyngeal cancer, head and neck squamous cell carcinoma, or prostate cancer).

The term “cancer” refers to all types of cancer, neoplasm or malignant tumors found in mammals, including leukemia, carcinomas and sarcomas. Exemplary cancers that may be treated with a compound or method provided herein include brain cancer, glioma, glioblastoma, neuroblastoma, prostate cancer, colorectal cancer, pancreatic cancer, medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with a compound or method provided herein include cancer of the thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer. In embodiments, the cancer or tumor type is adrenalcortical cancer, bladder/urothelial cancer, breast cancer, cervical cancer, cholangiocarcinoma, colorectal adenocarcinoma, diffuse large B-cell lymphoma, glioma, head and neck squamous cell carcinoma, renal cancer, renal clear cell cancer, papillary cell cancer, hepatocellular cancer, lung cancer, mesothelioma, ovarian cancer, pancreatic cancer, pheochromocytoma, paraganglioma, prostate cancer, rectal cancer, sarcoma, melanoma, stomach or esophageal cancer, testicular cancer, thyroid cancer, thymoma, uterine cancer, and/or uveal melanoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The terms “treating”, or “treatment” refers to any indicia of success in the therapy or amelioration of an injury, disease, pathology or condition, including any objective or subjective parameter such as abatement: remission: diminishing of symptoms or making the injury, pathology or condition more tolerable to the patient: slowing in the rate of degeneration or decline: making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms (e.g., ocular pain, seeing halos around lights, red eye, very high intraocular pressure), fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In instances, chronic administration may be required. For example, the compositions are administered to the subject in an amount and for a duration sufficient to treat the patient. In embodiments, the treating or treatment is not prophylactic treatment.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In embodiments, a patient is human.

A “effective amount”, as used herein, is an amount sufficient for a compound to accomplish a stated purpose relative to the absence of the compound (e.g., achieve the effect for which it is administered, treat a disease, reduce enzyme activity, increase enzyme activity, reduce a signaling pathway, or reduce one or more symptoms of a disease or condition). In these methods, the effective amount of the compound is an amount effective to accomplish the stated purpose of the method. An example of an “effective amount” is an amount sufficient to contribute to the treatment, prevention, or reduction of a symptom or symptoms of a disease, which could also be referred to as a “therapeutically effective amount,” A “reduction” of a symptom or symptoms (and grammatical equivalents of this phrase) means decreasing of the severity or frequency of the symptom(s), or elimination of the symptom(s). The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “−fold” increase or decrease. For example, a therapeutically effective amount can have at least a 1.2-fold, 1.5-fold, 2-fold, 5-fold, or more effect over a control.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

“Biological sample” is used in accordance with its plain and ordinary meaning and encompasses any sample type that can be used in a diagnostic, prognostic, or treatment method described herein. The biological sample may be any bodily fluid, tissue or any other sample obtained from a subject or subject's body from which clinically relevant protein marker levels or antibody levels may be determined. The definition encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents, solubilization, or enrichment for certain components, such as polypeptides or proteins. The term “biological sample” encompasses a clinical sample, but also, in embodiments, includes cells in culture, cell supernatants, cell lysates, blood, serum, plasma, urine, cerebral spinal fluid, biological fluid, and tissue samples. The sample may be pretreated as necessary by dilution in an appropriate buffer solution or concentrated, if desired. In embodiments, the biological sample is a blood sample. In embodiments, the biological sample is whole blood, plasma, or serum. In embodiments, the biological sample is a cancer cell. In embodiments, the biological sample is a cancer tumor.

“Control,” “suitable control,” or “control experiment” is used in accordance with its plain ordinary meaning and refers to an experiment in which the subjects or reagents of the experiment are treated as in a parallel experiment except for omission of a procedure, reagent, or variable of the experiment. In embodiments, the control is used as a standard of comparison in evaluating experimental effects. In embodiments, a control is the measurement of the activity of a protein in the absence of a compound as described herein (including embodiments and examples). For example, a test sample can be taken from a patient suspected of having a given disease (e.g., cancer) and compared to samples from a known cancer patient, or a known normal (non-disease) individual. A control can also represent an average value gathered from a population of similar individuals, e.g., cancer patients or healthy individuals with a similar medical background, same age, weight, etc. A control value can also be obtained from the same individual, e.g., from an earlier-obtained sample, prior to disease, or prior to treatment. One of skill will recognize that controls can be designed for assessment of any number of parameters. In embodiments, a control is a negative control. One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

### EMBODIMENTS

Embodiment 1. A compound of Formula (I):

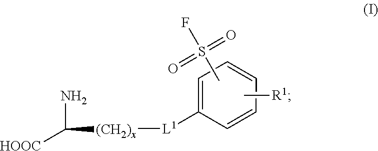

wherein: x is an integer from 0 to 8; L¹ is a bond, substituted or unsubstituted alkylene, or substituted or unsubstituted heteroalkylene; R¹ is halogen, —CX¹₃, —CHX¹₂, —CH₂X¹, —OCX¹₃, —OCH₂X¹, —OCHX¹₂, —CN, —SOₙ₁R^{1A}, —SOᵥ₁NR^{1A}R^{1B}, —NHC(O)NR^{1A}R^{1B}, —N(O)ₘ₁, —NR^{1A}R^{1B}, —C(O)R^{1A}, —C(O)—OR^{1A}, —C(O)NR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, substituted or unsubstituted alkyl, or substituted or unsubstituted heteroalkyl; X¹ is independently —F, —Cl, —Br, or —I; R^{1A} is hydrogen, substituted or unsubstituted alkyl, or substituted or unsubstituted heteroalkyl; R^{1B} is hydrogen, substituted or unsubstituted alkyl, or substituted or unsubstituted heteroalkyl; n1 is an integer from 0 to 4; m¹ is 1 or 2; and v1 is 1 or 2.
Embodiment 2. The compound of Embodiment 1, wherein x is an integer from 1 to 4.
Embodiment 3. The compound of Embodiment 1 or 2, wherein L¹ is a bond.
Embodiment 4. The compound of Embodiment 1 or 2, wherein L¹ is substituted or unsubstituted 2 to 6 membered heteroalkylene.
Embodiment 5. The compound of Embodiment 4, wherein L¹ is —NH—C(O)—(CH₂)_{y}— or —NH—C(O)—O—(CH₂)_{y}—, and y is an integer from 0 to 3.
Embodiment 6. The compound of any one of Embodiments 1 to 5, wherein R¹ is substituted or unsubstituted heteroalkyl.
Embodiment 7. The compound of Embodiment 6, wherein R¹ is unsubstituted 2 to 8 membered heteroalkyl.
Embodiment 8. The compound of Embodiment 7, wherein R¹ is —O—(CH₂)ₘCH₃, and m is an integer from 0 to 4.
Embodiment 9. The compound of any one of Embodiments 1 to 8, wherein R¹ is ortho to —S(═O)₂F.
Embodiment 10. The compound of any one of Embodiments 1 to 9, wherein the compound of Formula (I) is a compound of Formula (IA):

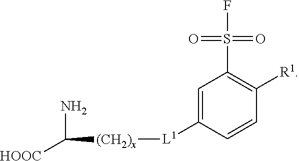

Embodiment 11. The compound of Embodiment 1, wherein the compound of Formula (I) is a compound of Formula (IB):

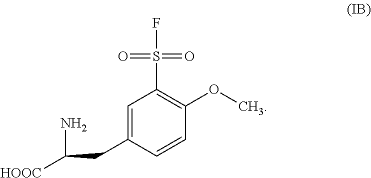 [SMILES: COc1ccc(C[C@H](N)C(=O)O)cc1S(=O)(=O)F]

Embodiment 12. A biomolecule comprising an unnatural amino acid, wherein the unnatural amino comprises a side chain of Formula (II):

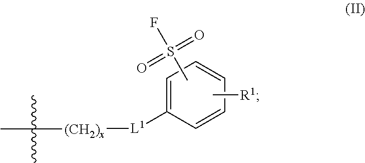

wherein: x is an integer from 1 to 8; L¹ is a bond, substituted or unsubstituted alkylene, or substituted or unsubstituted heteroalkylene; R¹ is halogen, —CX¹₃, —CHX¹₂, —CH₂X¹, —OCX¹₃, —OCH₂X¹, —OCHX¹₂, —CN, —SOₙ₁R^{1A}, —SOᵥ₁NR^{1A}R^{1B}, —NHC(O)NR^{1A}R^{1B}, —N(O)ₘ₁, —NR^{1A}R^{1B}, —C(O)R^{1A}, —C(O)—OR^{1A}, —C(O)NR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, substituted or unsubstituted alkyl, or substituted or unsubstituted heteroalkyl; X¹ is independently —F, —Cl, —Br, or —I; R^{1A} is hydrogen, substituted or unsubstituted alkyl, or substituted or unsubstituted heteroalkyl; R^{1B} is hydrogen, substituted or unsubstituted alkyl, or substituted or unsubstituted heteroalkyl; n1 is an integer from 0 to 4; m¹ is 1 or 2; and v1 is 1 or 2.
Embodiment 13. The biomolecule of Embodiment 12, wherein x is an integer from 1 to 4.
Embodiment 14. The biomolecule of Embodiment 12 or 13, wherein L¹ is a bond.
Embodiment 15. The biomolecule of Embodiment 12 or 13, wherein L¹ is substituted or unsubstituted 2 to 6 membered heteroalkylene.
Embodiment 16. The biomolecule of Embodiment 15, wherein L¹ is —NH—C(O)—(CH₂)_{y}— or —NH—C(O)—O—(CH₂)_{y}—, and y is an integer from 0 to 2.
Embodiment 17. The biomolecule of any one of Embodiments 12 to 16, wherein R¹ is substituted or unsubstituted heteroalkyl.
Embodiment 18. The biomolecule of Embodiment 17, wherein R¹ is unsubstituted 2 to 8 membered heteroalkyl.
Embodiment 19. The biomolecule of Embodiment 18, wherein R¹ is —O—(CH₂)ₘCH₃, and m is an integer from 0 to 4.
Embodiment 20. The biomolecule of any one of Embodiments 12 to 19, wherein R¹ is ortho to —S(—O)₂F.
Embodiment 21. The biomolecule of any one of Embodiments 12 to 20, wherein the side chain of Formula (II) has the structure of Formula (IIA):

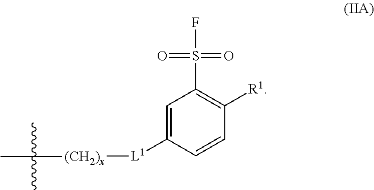

Embodiment 22. The biomolecule of Embodiment 12, wherein the side chain of Formula (II) has the structure of Formula (IIB):

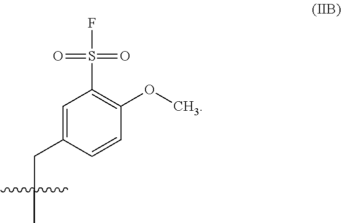 [SMILES: *Cc1ccc(OC)c(S(=O)(=O)F)c1]

Embodiment 23. The biomolecule of any one of Embodiments 12 to 22, wherein the biomolecule comprises a lipid or RNA.
Embodiment 24. The biomolecule of any one of Embodiments 12 to 22, wherein the biomolecule comprises a protein.
Embodiment 25. The biomolecule of Embodiment 24, wherein the protein comprises a glycan-binding protein which comprises the unnatural amino acid.
Embodiment 26. The biomolecule of Embodiment 25, wherein the glycan-binding protein is a sialic acid-binding immunoglobulin-type lectin (Siglec) which comprises the unnatural amino acid or a sialoglycan binding V-set domain of sialic acid-binding immunoglobulin-type lectin (Siglec) which comprises the unnatural amino acid.
Embodiment 27. The biomolecule of Embodiment 26, wherein the Siglec is Siglec-1, Siglec-2, Siglec-3, Siglec-4, Siglec-5, Siglec-6, Siglec-7, Siglec-8, Siglec-9, Siglec-10, Siglec-11, Siglec-12, Siglec-14, or Siglec-15.
Embodiment 28. The biomolecule of Embodiment 27, wherein the Siglec is Siglec-7.
Embodiment 29. The biomolecule of Embodiment 28, wherein the Siglec-7 has at least 85% sequence identity to SEQ ID NO:1, SEQ ID NO:2, SEQ ID NO:3, or SEQ ID NO:4.
Embodiment 30. The biomolecule of any one of Embodiments 26 or 29, wherein the side chain is at a lysine residue at a position corresponding to position 104 or position 127; or wherein the side chain is at an asparagine residue at a position corresponding to position 129.
Embodiment 31. The biomolecule of Embodiment 24, wherein the protein comprises a RNA-binding protein which comprises the unnatural amino acid.
Embodiment 32. The biomolecule of Embodiment 24, wherein the protein comprises a N⁶-methyladenosine reader protein which comprises the unnatural amino acid.
Embodiment 33. A nucleic acid encoding the biomolecule of any one of Embodiments 12 to 32.
Embodiment 34. A vector comprising the nucleic acid sequence of Embodiment 33.
Embodiment 35. A biomolecule conjugate of Formula (III):

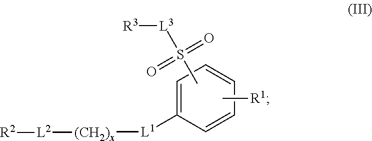

wherein: R² is a first biomolecule moiety; R³ is a second biomolecule moiety; L¹ is a bond, substituted or unsubstituted alkylene, or substituted or unsubstituted heteroalkylene; x is an integer from 1 to 8; R¹ is halogen, —CX¹₃, —CHX¹₂, —CH₂X¹, —OCX¹₃, —OCH₂X¹, —OCHX¹₂, —CN, —SOₙ₁R^{1A}, —SOₙ₁NR^{1A}R^{1B}, —NHC(O)NR^{1A}R^{1B}. —N(O)ₘ₁, —NR^{1A}R^{1B}, —C(O)R^{1A}, —C(O)—OR^{1A}, —C(O)NR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, substituted or unsubstituted alkyl, or substituted or unsubstituted heteroalkyl; X¹ is independently —F, —Cl, —Br, or —I; R^{1A} is hydrogen, substituted or unsubstituted alkyl, or substituted or unsubstituted heteroalkyl; R^{1B} is hydrogen, substituted or unsubstituted alkyl, or substituted or unsubstituted heteroalkyl; n1 is an integer from 0 to 4; m¹ is 1 or 2; v1 is 1 or 2; L² is a bond, —NR^{2A}—, —S—, —S(O)₂—, —O—, —C(O)—, —C(O)O—, —OC(O)—, —N(R^{2A})C(O)—, —C(O)N(R^{2A})—, —NR^{2A}C(O)NR^{2B}—, —NR^{2A}C(NH)NR^{2B}—, —SO₂N(R^{2A})—, —N(R^{2A})SO₂—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; L³ is a bond, —N(R^{3A})—, —S—, —S(O)₂—, —O—, —C(O)—, —C(O)O—, —OC(O)—, —N(R^{3A})C(O)—, —C(O)N(R^{3A})—, —NR^{3A}C(O)NR^{3B}—, —NR^{3A}C(NH)NR^{3B}—, —SO₂N(R^{3A})—, —N(R^{3A})SO₂—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and R^{2A}, R^{2B}, R^{3A}, and R^{3B} are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.
Embodiment 36. The biomolecule conjugate of Embodiment 35, wherein x is an integer from 1 to 4.
Embodiment 37. The biomolecule conjugate of Embodiment 35 or 36, wherein L¹ is a bond.
Embodiment 38. The biomolecule conjugate of Embodiment 35 or 36, wherein L¹ is substituted or unsubstituted 2 to 6 membered heteroalkylene.
Embodiment 39. The biomolecule conjugate of Embodiment 38, wherein L¹ is —NH—C(O)—(CH₂)_{y}— or —NH—C(O)—O—(CH₂)_{y}—, and y is an integer from 0 to 2.
Embodiment 40. The biomolecule conjugate of any one of Embodiments 35 to 39, wherein R¹ is substituted or unsubstituted heteroalkyl.
Embodiment 41. The biomolecule conjugate of Embodiment 40, wherein R¹ is unsubstituted 2 to 8 membered heteroalkyl.
Embodiment 42. The biomolecule conjugate of Embodiment 41, wherein R¹ is —O—(CH₂)ₘCH₃, and m is an integer from 0 to 4.
Embodiment 43. The biomolecule conjugate of any one of Embodiments 35 to 42, wherein R¹ is ortho to —S(═O)₂F.
Embodiment 44. The biomolecule conjugate of any one of Embodiments 35 to 43, wherein: L² is a bond, —NH—, —S—, —S(O)₂—, —O—, —C(O)—, —C(O)O—, —OC(O)—, —NHC(O)—, —C(O)NH—, —NHC(O)NH—, —NHC(NH)NH—, —SO₂NH—, —NHSO₂—, —C(S)—, L¹²-substituted or unsubstituted alkylene, L¹²-substituted or unsubstituted heteroalkylene, L¹²-substituted or unsubstituted cycloalkylene, L¹²-substituted or unsubstituted heterocycloalkylene, L¹²-substituted or unsubstituted arylene, or L¹²-substituted or unsubstituted heteroarylene; L¹² is halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —N(O)₂, —NHSO₂H, —NHC(O)H, —NHC(O)OH, —NHOH, —N₃, unsubstituted alkyl, unsubstituted heteroalkyl, unsubstituted cycloalkyl, unsubstituted heterocycloalkyl, unsubstituted aryl, or unsubstituted heteroaryl; L³ is a bond, —NH—, —S—, —S(O)₂—, —O—, —C(O)—, —C(O)O—, —OC(O)—, —NHC(O)—, —C(O)NH—, —NHC(O)NH—, —NHC(NH)NH—, —SO₂NH—, —NHSO₂—, —C(S)—, L¹³-substituted or unsubstituted alkylene, L¹³-substituted or unsubstituted heteroalkylene, L¹³-substituted or unsubstituted cycloalkylene, L¹³-substituted or unsubstituted heterocycloalkylene, L¹³-substituted or unsubstituted arylene, or L¹²-substituted or unsubstituted heteroarylene; and L¹³ is halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —N(O)₂, —NHSO₂H, —NHC(O)H, —NHC(O)OH, —NHOH, —N₃, unsubstituted alkyl, unsubstituted heteroalkyl, unsubstituted cycloalkyl, unsubstituted heterocycloalkyl, unsubstituted aryl, or unsubstituted heteroaryl.
Embodiment 45. The biomolecule conjugate of any one of Embodiments 35 to 44 wherein the biomolecule conjugate of Formula (III) is a biomolecule conjugate of Formula (IIIA):

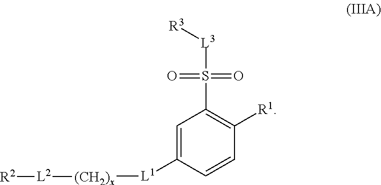

Embodiment 46. The biomolecule conjugate of Embodiment 45, wherein the biomolecule conjugate of Formula (IIIA) is a biomolecule conjugate of Formula (IIIB):

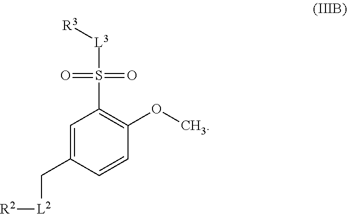

Embodiment 47. The biomolecule conjugate of any one of Embodiments 35 to 46, wherein L² is a bond and L³ is a bond.
Embodiment 48. The biomolecule conjugate of any one of Embodiments 35 to 47, wherein R² is a peptidyl moiety, a lipid moiety, an RNA moiety, or a glycan moiety, and R³ is a peptidyl moiety, a lipid moiety, a RNA moiety, or a glycan moiety.
Embodiment 49. The biomolecule conjugate of any one of Embodiments 35 to 47, wherein R² is a peptidyl moiety, a lipid moiety, or a glycan moiety, and R³ is a RNA moiety.
Embodiment 50. The biomolecule conjugate of any one of Embodiments 35 to 47, wherein R² is a peptidyl moiety, and R³ is a RNA moiety.
Embodiment 51. The biomolecule conjugate of Embodiment 50, wherein the peptidyl moiety is a RNA-binding peptidyl moiety.
Embodiment 52. The biomolecule conjugate of Embodiment 50, wherein the peptidyl moiety is a N⁶-methyladenosine reader protein moiety.
Embodiment 53. The biomolecule conjugate of any one of Embodiments 50 to 52, wherein L³ is bonded to a N⁶-methyladenosine residue on the RNA moiety.
Embodiment 54. The biomolecule conjugate of Embodiment 53, wherein L³ is a bond.
Embodiment 55. The biomolecule conjugate of any one of Embodiments 35 to 47, wherein R² is a peptidyl moiety, a lipid moiety, or an RNA moiety, and R³ is a glycan moiety.
Embodiment 56. The biomolecule conjugate of any one of Embodiments 35 to 47, wherein R² is a peptidyl moiety and R³ is a glycan moiety.
Embodiment 57. The biomolecule conjugate of Embodiment 56, wherein R² is a glycan-binding peptidyl moiety and R³ is a glycan moiety.
Embodiment 58. The biomolecule conjugate of Embodiment 57, wherein the glycan-binding peptidyl moiety comprises a sialic acid-binding immunoglobulin-type lectin (Siglec) which comprises the unnatural amino acid; and wherein the glycan moiety comprises a sialoglycan.
Embodiment 59. The biomolecule conjugate of Embodiment 57, wherein the peptidyl moiety comprises a sialoglycan binding V-set domain of sialic acid-binding immunoglobulin-type lectin (Siglec) which comprises the unnatural amino acid; and wherein the glycan moiety comprises a sialoglycan.
Embodiment 60. The biomolecule conjugate of Embodiment 58 or 59, wherein the Siglec is Siglec-1, Siglec-2, Siglec-3, Siglec-4, Siglec-5, Siglec-6, Siglec-7, Siglec-8, Siglec-9, Siglec-10, Siglec-11, Siglec-12, Siglec-14, or Siglec-15.
Embodiment 61. The biomolecule conjugate of Embodiment 60, wherein the Siglec is Siglec-7.
Embodiment 62. The biomolecule conjugate of Embodiment 61, wherein Siglec-7 has at least 85% sequence identity to SEQ ID NO: 1, SEQ ID NO:2, SEQ ID NO:3, or SEQ ID NO:4.
Embodiment 63. The biomolecule conjugate of Embodiment 62, wherein R² is bonded to L² at a lysine residue at a position corresponding to position 104 or position 127; or wherein R² is bonded to L² at an asparagine residue at a position corresponding to position 129.
Embodiment 64. The biomolecule conjugate of any one of Embodiments 58 to 63, wherein the sialoglycan is bonded to L³ via an oxygen atom within the sialoglycan.
Embodiment 65. The biomolecule conjugate of Embodiment 64, wherein L³ is a bond.
Embodiment 66. The biomolecule conjugate of any one of Embodiments 55 to 65, wherein the glycan moiety is further bonded to a lipid, a protein, or RNA.
Embodiment 67. The biomolecule conjugate of Embodiment 66, wherein the glycan moiety is bonded to a cell membrane lipid.
Embodiment 68. The biomolecule conjugate of Embodiment 67, wherein the cell membrane lipid is a cancer cell membrane lipid.
Embodiment 69. A pyrrolysyl-tRNA synthetase comprising at least 6 amino acid residues substitutions within the substrate-binding site of the pyrrolysyl-tRNA synthetase having at least 85% sequence identity to the amino acid sequence of SEQ ID NO:5; wherein the substrate-binding site comprises residues tyrosine at position 306, leucine at position 309, asparagine at position 346, cysteine at position 348, and tryptophan at position 417 as set forth in the amino acid sequence of SEQ ID NO:5.
Embodiment 70. The pyrrolysyl-tRNA synthetase of Embodiment 69, wherein the at least 6 amino acid residues substitutions in the amino acid sequence of SEQ ID NO:5 are: (i) Y306L; (ii) L309A; (iii) N346A; (iv) C348M; and (v) W417T.
Embodiment 71. A pyrrolysyl-tRNA synthetase comprising at least 6 amino acid residues substitutions within the substrate-binding site of the pyrrolysyl-tRNA synthetase having at least 85% sequence identity to the amino acid sequence of SEQ ID NO:7; wherein the substrate-binding site comprises residues tyrosine at position 126, methionine at position 129, asparagine at position 166, valine at position 168, and tryptophan at position 239 as set forth in the amino acid sequence of SEQ ID NO:7.
Embodiment 72. The pyrrolysyl-tRNA synthetase of Embodiment 71, wherein the at least 6 amino acid residues substitutions in the amino acid sequence of SEQ ID NO:7 are: (i) Y126L; (ii) M129A; (iii) N166A; (iv) V168M; and (v) W239T.
Embodiment 73. A nucleic acid encoding the pyrrolysyl-tRNA synthetase of any one of Embodiments 69 to 72.
Embodiment 74. A vector comprising the nucleic acid of Embodiment 73.
Embodiment 75. The vector of Embodiment 74, further comprising a nucleic acid encoding tRNA^{Pyl}.
Embodiment 76. A complex comprising the pyrrolysyl-tRNA synthetase of any one of Embodiments 69 to 72 and the compound of any one of Embodiments 1 to 11.
Embodiment 77. The complex of Embodiment 76, further comprising a tRNA^{Pyl}.
Embodiment 78. A cell comprising: (i) the compound of any one of Embodiments 1 to 11; (ii) the biomolecule of any one of Embodiments 12 to 32; (iii) the nucleic acid of Embodiment 33 or 73; (iv) the vector of Embodiment 34, 74, or 75; (v) the biomolecule conjugate of any one of Embodiments 35 to 68; (vi) the pyrrolysyl-tRNA synthetase of any one of Embodiments 69 to 72; or (vii) the complex of Embodiment 76 or 77.
Embodiment 79. The cell of Embodiment 78, wherein the cell is a bacterial cell or a mammalian cell.
Embodiment 80. A pharmaceutical composition comprising the biomolecule of any one of Embodiments 12 to 32, the nucleic acid of Embodiment 33, or the vector of Embodiment 34, and a pharmaceutically acceptable excipient.
Embodiment 81. A method of treating cancer in a patient in need thereof, the method comprising administering to the patient an effective amount of the biomolecule of any one of Embodiments 12 to 32, the nucleic acid of Embodiment 33, the vector of Embodiment 34, or the pharmaceutical composition of Embodiment 80.
Embodiment 82. The method of Embodiment 77, comprising administering to the patient an effective amount of the biomolecule of any one of Embodiments 26 to 32.
Embodiment 83. The method of Embodiment 81 or 82, wherein the cancer is melanoma or breast cancer.
Embodiment 84. The method of any one of Embodiments 81 to 83, wherein the cancer comprises a sialoglycan.
Embodiment 85. The method of any one of Embodiments 81 to 84, wherein the cancer comprises an elevated level of sialoglycan relative to a control.
Embodiment 86. The method of any one of Embodiments 81 to 85, further comprising detecting an elevated level of sialoglycan in a biological sample obtained from the patient.
Embodiment 87. A method of identifying a N⁶-methyladenosine site on RNA, the method comprising contacting the biomolecule of Embodiment 32 with the RNA, thereby identify the N⁶-methyladenosine site.
Embodiment 88. A method of identifying a N⁶-methyladenosine site on RNA, the method comprising contacting the biomolecule of Embodiment 24 with the RNA, wherein the protein is a N⁶-methyladenosine demethylase protein, thereby identifying the N⁶-methyladenosine site.
Embodiment 89. The method of Embodiment 87 or 88, wherein the RNA is in the transcriptome.
Embodiment 90. The biomolecule of Embodiment 24, wherein the protein comprises a N⁶-methyladenosine demethylase protein which comprises the unnatural amino acid.
Embodiment 91. The biomolecule conjugate of Embodiment 50, wherein the peptidyl moiety is a N⁶-methyladenosine demethylase protein moiety.

### EXAMPLES

The following examples are intended to further illustrate certain embodiments of the disclosure. The examples are put forth so as to provide one of ordinary skill in the art and are not intended to limit its scope.

Recent success in creating covalent linkages between proteins in vivo has enabled the capture of elusive protein-protein interactions as well as the development of covalent protein drugs for cancer immunotherapy. (Refs. 9-12). However, glycans contain mainly the weak nucleophilic hydroxyl group, which are difficult to react with under mild aqueous conditions. Unlike amino acid residues of proteins, many of which have distinct functional groups in their side chains to distinguish, the functional groups of monosaccharides are more or less the same. Efficient differentiation between the multiple hydroxyl groups of an unprotected glycan is also difficult without enzyme catalysis. (Ref. 13). These chemical features of glycan not only make them challenging to synthesize but also to selectively target with biocompatible chemistry. (Ref. 14).

Sialic acid-binding immunoglobulin-like lectin 7 (Siglec-7) is an inhibitory transmembrane receptor mainly expressed on human natural killer (NK) cells. (Ref. 15). Siglec-7 recognizes sialic acid via its extracellular V-set immunoglobulin domain and signals through its cytosolic immunoreceptor tyrosine-based inhibitory motif (ITIM) to attenuate NK cell activation. (Ref. 16). The preferred glycan ligand for Siglec-7 is Neu5 Acα2-8Neu5Ac-containing glycans with generally low binding affinity. (Refs. 17-18). Siglec-7 natively contributes to the discrimination between self and non-self, but some pathogens and cancers can up-regulate sialoglycan to evade immune surveillance and NK cell-mediated killing. (Ref. 19). New strategies to block such exploitation would be valuable for developing glycan-based immunotherapy.

### Example 1

Here we developed a biocompatible method, genetically encoded chemical cross-linking of proteins with sugar (GECX-sugar), to generate covalent linkages between proteins and glycans with residue specificity. We identified that sulfonyl fluoride was able to cross-link sugar via proximity-enabled reactivity, and genetically encoded into proteins a novel bioreactive unnatural amino acid (Uaa) SFY containing the sulfonyl fluoride. The SFY-incorporated Siglec-7 covalently and specifically cross-linked its substrate sialoglycan in vitro and on cancer cell surface. Moreover, through covalent binding with sialoglycan on cancer cell surface, SFY-incorporated Siglec-7 enhanced the killing of cancer cells by natural killer (NK) cells. The site-specific covalent linkage between protein and glycan enabled by GECX-sugar will facilitate the study of protein-glycan interactions and open new avenues for engineering covalent protein-glycan complex for research and therapeutic purposes.

### Results

### Identification of Sulfonyl Fluoride to Cross-Link Glycan Using Plant-and-Cast Cross-Linkers

It is challenging to covalently target glycan under mild physiological conditions. The dominant functional group, the hydroxyl group, is a weak nucleophile and difficult to chemically differentiate from water. Recently we have succeeded in covalently targeting amino acid side chains and RNA nucleotides in vivo via proximity-enabled reactivity (Ref. 9), we thus expect that glycan could also be covalently targeted via this mechanism.

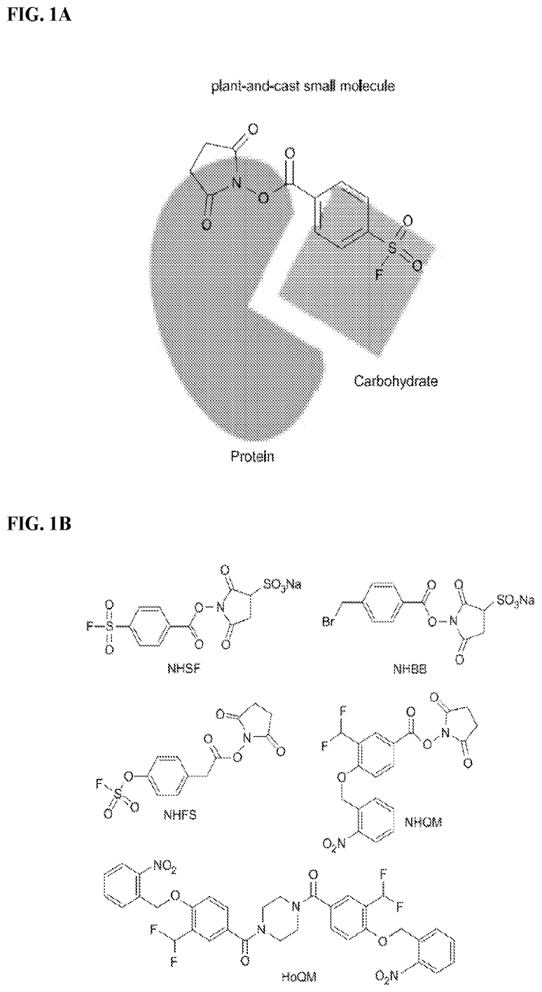

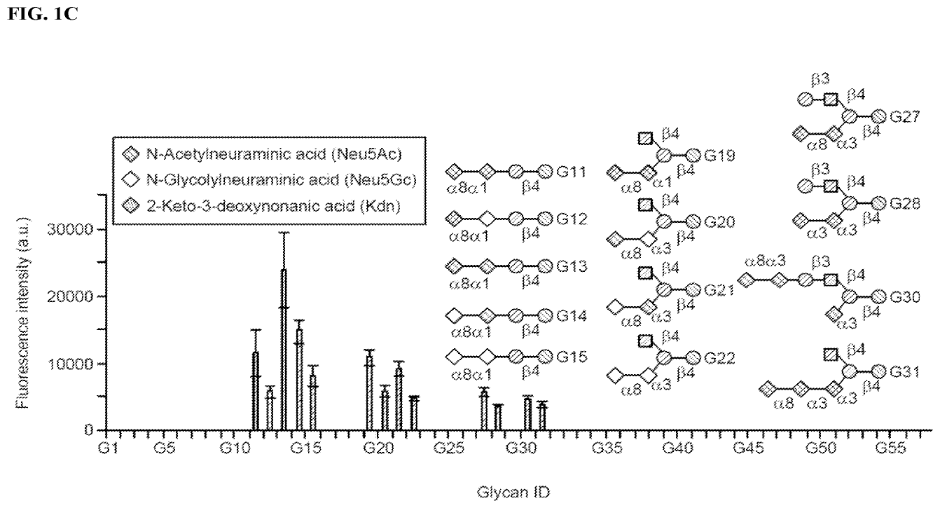

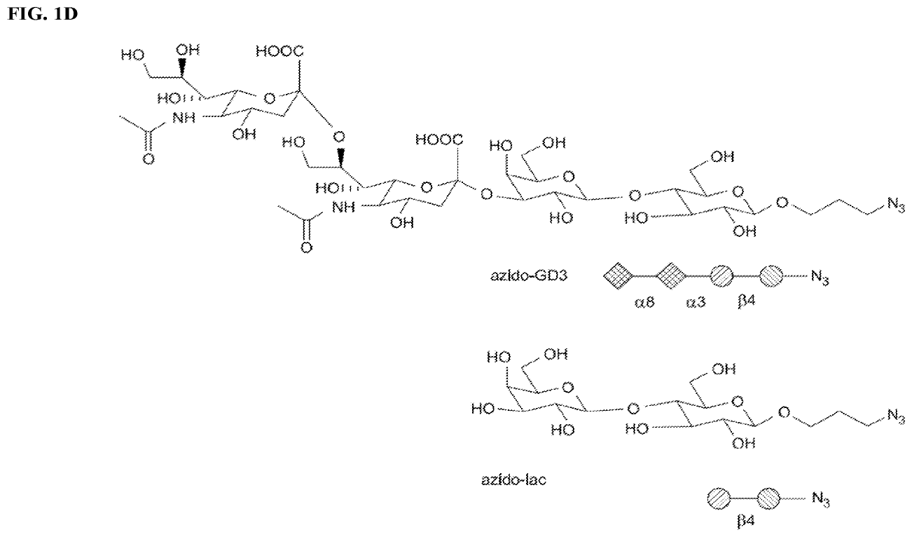

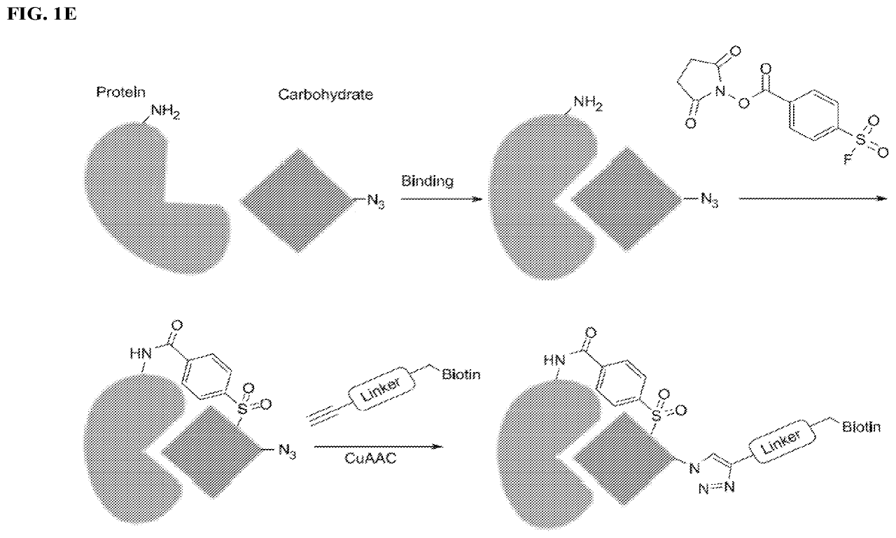

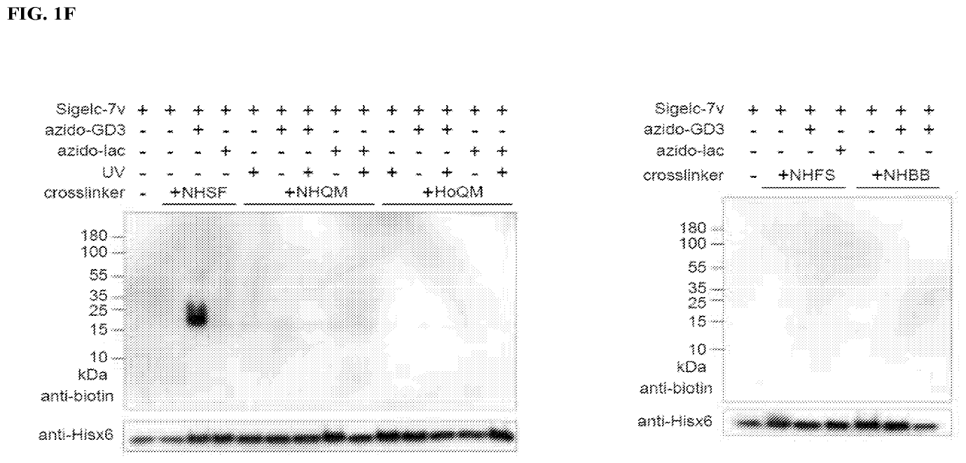

To identify functional groups that react with glycan with reactivity driven by proximity effect, we developed a strategy involving the use of plant-and-cast small molecule cross-linkers to cross-link protein-glycan complex (FIG. 1A). The plant-and-cast small molecule cross-linkers contain a highly reactive succinimide ester, which reacts with Lys side chain and plants the cross-linker on proteins. (Ref. 20). The other end of the cross-linker contains a less reactive functional group and is cast to react with nearby functional groups of protein or glycan. The covalent cross-link of protein with the bound glycan can be readily determined with Western blot under denatured conditions, indicating that the functional group on the cross-linker is able to react with glycan.

Based on our experience with targeting amino acid side chains via proximity-enabled reactivity, we designed and synthesized five plant-and-cast cross-linkers containing sulfonyl fluoride, benzyl bromide, fluorosulfate, and photocaged quinone methide (QM), respectively (FIG. 1B). (Refs. 20-27). Functional groups of NHSF, NHBB, and NHFS are relatively inert but can react with nucleophilic side chains of Lys, His, or Tyr in close proximity. NHQM, upon photo-activation, generates QM, which is able to react with nine nucleophilic amino acid side chains including the very weak ones of Gln and Asn. (Ref. 26). The fifth cross-linker, HoQM, was also tested since it has photocage QM at both ends (Ref. 28), which may increase the planting sites on protein and thus casting more QM to the glycan for reaction.

To cross-link protein-glycan interactions, we chose to work with Siglec-7, a transmembrane receptor expressed on human immune cells to regulate immune function through recognizing sialoglycans. We cloned and expressed the extracellular, sialoglycan binding V-set domain of Siglec-7 in E. coli, referred to herein as Siglec-7v or SEQ ID NO:3. The Siglec-7v was purified from inclusion bodies in high concentrations of guanidine and refolded using step-wise dialysis. The intact Siglec-7v was analyzed with electrospray ionization time-of-flight mass spectrometry (ESI-TOF MS). A major peak was observed at 15937.5 Da, corresponding to intact Siglec-7v with disulfide bond formed. A glycosphingolipid glycan microarray featuring 58 glycan epitopes was used for functional analysis of the refolded Siglec-7v (FIG. 10). Consistent with Siglec-7v expressed from mouse cells, the refolded Siglec-7v bound with glycan ligands containing terminal 2,8-linked sialic acid only, and not with other glycan ligands lacking terminal sialic acid (FIG. 1C). In addition, among these bound glycans, higher binding affinity was measured with the linear Neu5Acα2-8Neu5 Ac-terminating ligands (FIGS. 1C, 10, G11-G15) over the asymmetrically branched Neu5 Acα2-8Neu5Ac-terminating ligands (FIGS. 1C, 10, G19-G22, G27-G31). These results confirmed that the Siglec-7v expressed as inclusion bodies in E. coli was properly refolded and functional, i.e., had the correct binding properties.

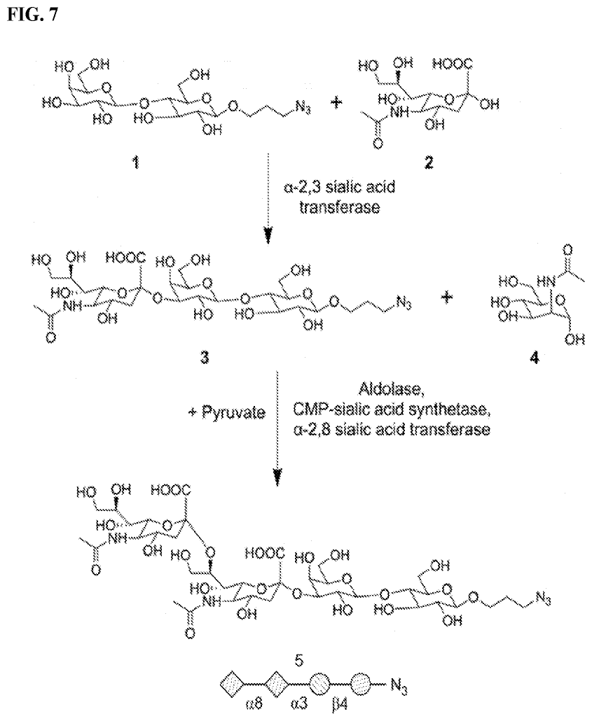

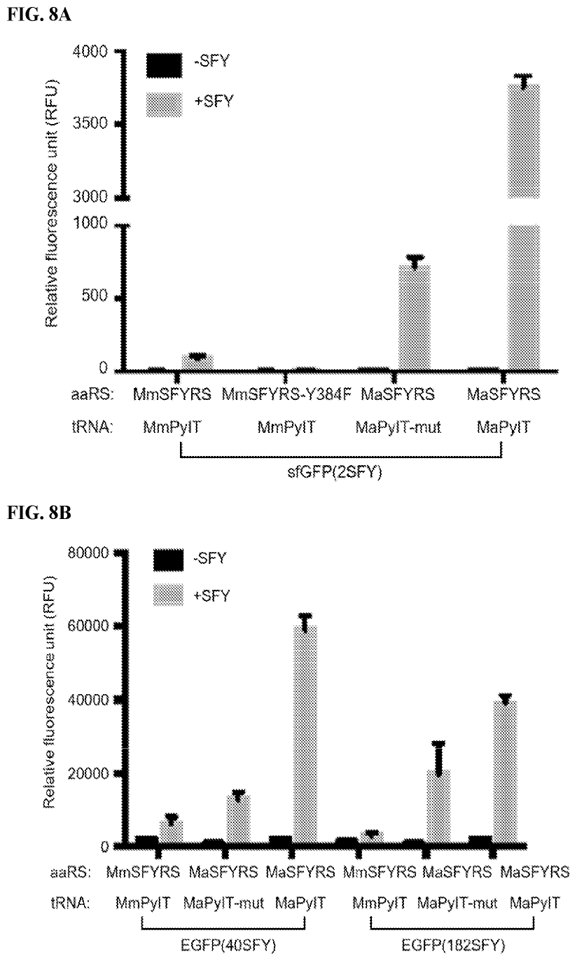

From the binding assay of Siglec-7v, we chose G11 as the model ligand for Siglec-7v cross-linking study. G11 was called GD3 ganglioside sugar, which is a tumor-associated glycan antigen. (Refs. 29-31). To facilitate identification of GD3, we added an azido group at the lactose terminal, referred to as azido-GD3 (FIG. 1D), which was synthesized with a chemo-enzymatic method (FIG. 7), purified using HPLC, and verified with ESI-MS. As a negative control for binding with Siglec-7v, an azido-lactose (azido-lac) was also synthesized, which lacked the two terminal Neu5Acα2-8Neu5Ac compared with azido-GD3 (FIG. 1D).

We then investigated if the various function groups synthesized onto the cross-linkers (FIG. 1B) could achieve protein-glycan cross-linking using Siglec-7v and azido-GD3 as the model system. Siglec-7v (60 μM) was incubated with 2 mM azido-GD3 for 1 h at room temperature, followed by the addition of different cross-linkers to the solution. For samples treated with cross-linker NHQM or HoQM, UV light was applied to activate the cross-linker. Alkyne-biotin was subsequently clicked with azido-GD3 in the samples, and the biotin signal was detected with Western blot using streptavidin-HRP (FIG. 1E). A strong band with molecular weight slightly above Siglec-7v was clearly detected only in samples treated with NHSF (FIG. 1F), but not with the other four cross-linkers (i.e., NHBB, NHFS, NHQM, HoQM). We previously demonstrated that NHSF could form cross-links between Lys residues and His, Ser, Thr, Tyr, and Lys side chains within and between proteins. (Ref. 20). The results here indicate that NHSF can be applied to cross-link protein-glycan interactions via the sulfonyl fluoride group.

### NHSF Covalently Targets Specific Protein-Glycan Interactions Via Proximity-Enabled Reactivity

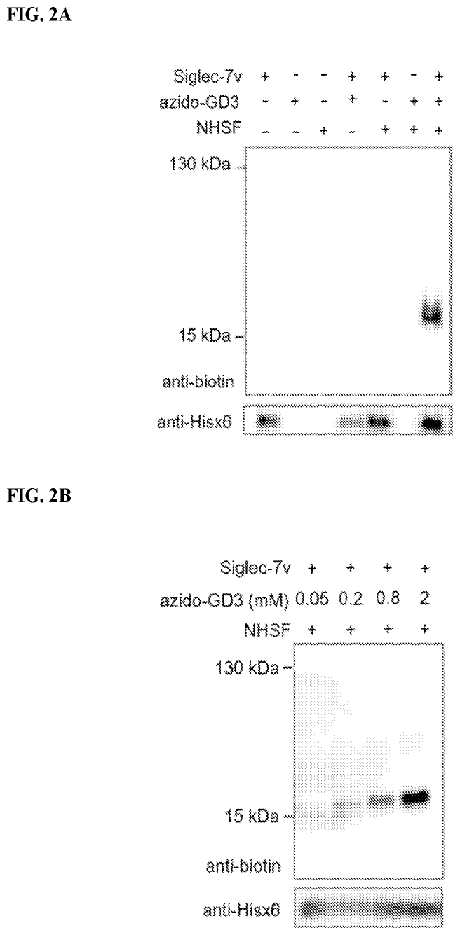

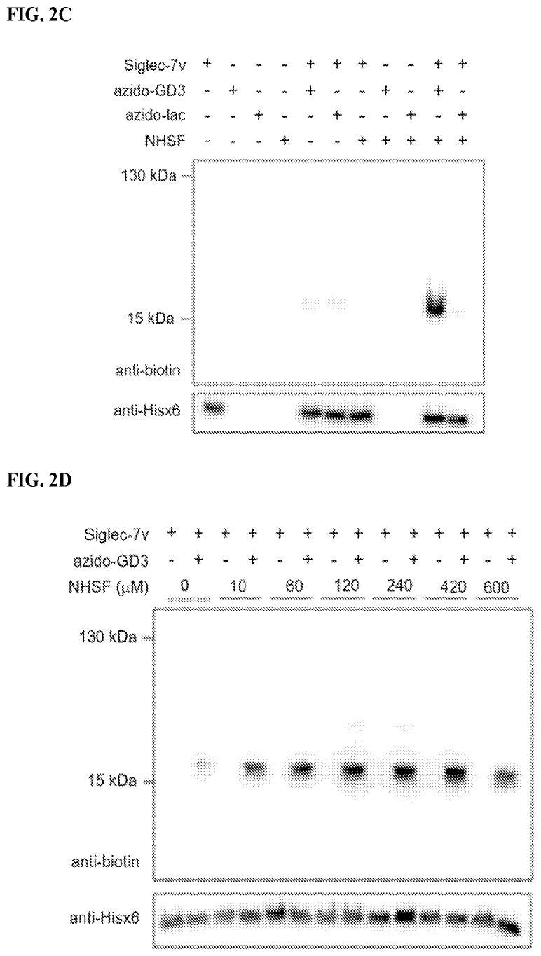

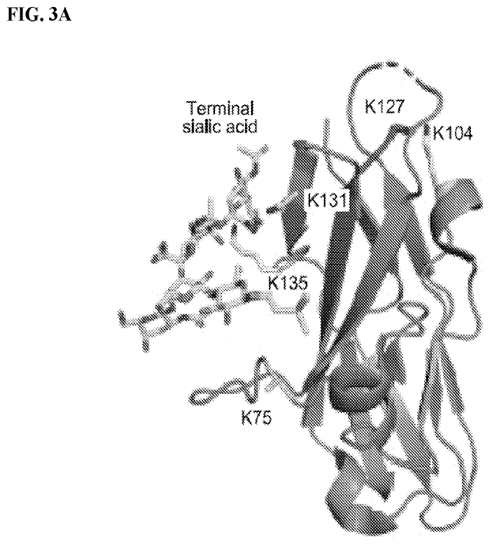

To study the specificity of NHSF-mediated glycan cross-linking, we investigated the cross-linking of Siglec-7v with azido-GD3 by NHSF in more detail. The cross-linking band was observed only when Siglec-7v, azido-GD3, and NHSF were all present: no cross-linking was detected with either one or two components withdrawn from incubation (FIG. 2A). The minimum azido-GD3 concentration for cross-linking with 60 μM Siglec-7v was around 0.8 mM (FIG. 2B), consistent with the reported low binding affinity of Siglec-7 for sialoglycan. In addition, when the negative control glycan ligand, azido-lac, was used in place of azido-GD3, the cross-linking band with Siglec-7v was no longer detected (FIG. 2C). These results indicated that cross-linking by NHSF would only occur when Siglec-7v was bound with its glycan substrate. Therefore. NHSF cross-linked specific protein-glycan interactions, presumably because sulfonyl fluoride is a relatively weak electrophile and protein-glycan interactions, which put the sulfonyl fluoride and glycan in close proximity, triggered the cross-linking reaction. Various concentrations of NHSF ranging from 10 μM to 600 μM enable the specific cross-linking of Siglec-7v with azido-GD3 (FIG. 2D), and cross-linking efficiency was dependent on NHSF concentration applied.

To assess if NHSF cross-linking of glycan was distance dependent, we first determined which Lys residue of Siglec-7v was NHSF planted on via the succinimide ester to cross-link azido-GD3. We individually mutated all Lys residues on the Siglec-7v to Gly (FIG. 3A)(Ref. 32), and tested cross-linking of these Siglec-7v mutants with azido-GD3 by NHSF. Mutation of Lys to Gly at the right site would abolish the NHSF planting on Siglec-7v to reach and cross-link with azido-GD3. Mutations at sites Lys20, 24, 75, 104, or 129 did not affect cross-linking of azido-GD3 to the Siglec-7v, while mutations at Lys127 and Lys135 showed negligible cross-linking (FIG. 3B). Previously, Lys135 has been shown to directly interact with the glycan ligand via hydrogen bonding, and mutation of Lys135 to Ala abolishes glycan ligand binding to Siglec-7. (Ref. 32). Our results of Siglec-7v(Lys135Gly) mutant consistently confirm the direct binding role of Lys135 and further support NHSF cross-linking of azido-GD3 is dependent on specific binding of the glycan to the receptor. Excluding Lys135. Lys127 thus should be the site on which NHSF was planted to cross-link azido-GD3. NHSF has been shown to cross-link Cα-Cα of Lys and Ser on BSA protein at a distance of 8.9 to 21.2 Å, and Lys127 on Siglec-7v showed reachable distance to the terminal sialic acid, which was about 14 Å (FIG. 3A). (Ref. 20). Therefore, these results indicate that NHSF cross-linked azido-GD3 in close distance via proximity-enabled reactivity.

To further evaluate distance dependence of NHSF cross-linking and to optimize cross-linking efficiency, we next altered the length of the cross-linker. Three additional analogs of NHSF were synthesized with increasing numbers of methylene in the linker to study the effects of linker length and flexibility on the cross-linking efficiency (FIG. 3C). Siglec-7v was incubated with either azido-GD3 or azido-lac with different cross-linkers. Western blot analysis of the incubation products showed that NHSF—C₂ increased the cross-linking band intensity 1.5-fold in comparison with NHSF (FIG. 3D). However, further increase of the linker length in NHSF—C₃ and NHSF—C₇ lowered the cross-linking efficiency. These results further corroborate the distance dependence and proximity-driven nature of NHSF cross-linking of azido-GD3.

Design and Genetic Incorporation of SFY into Proteins in E. coli

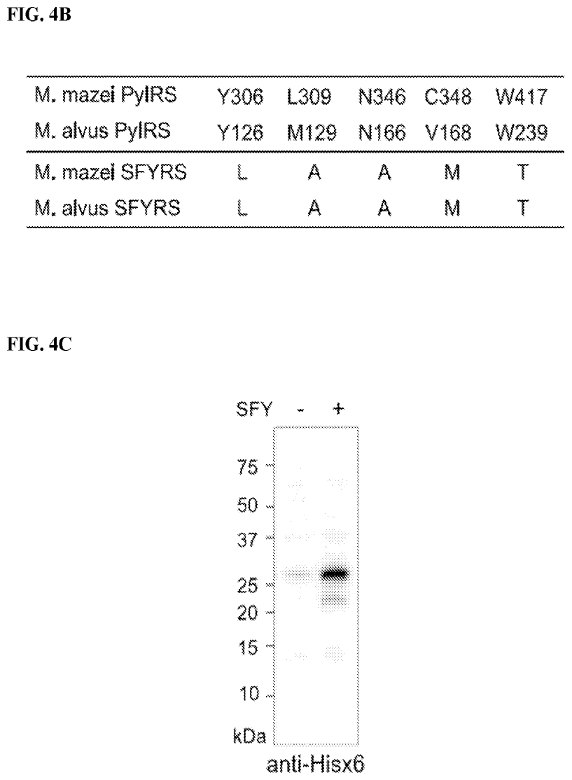

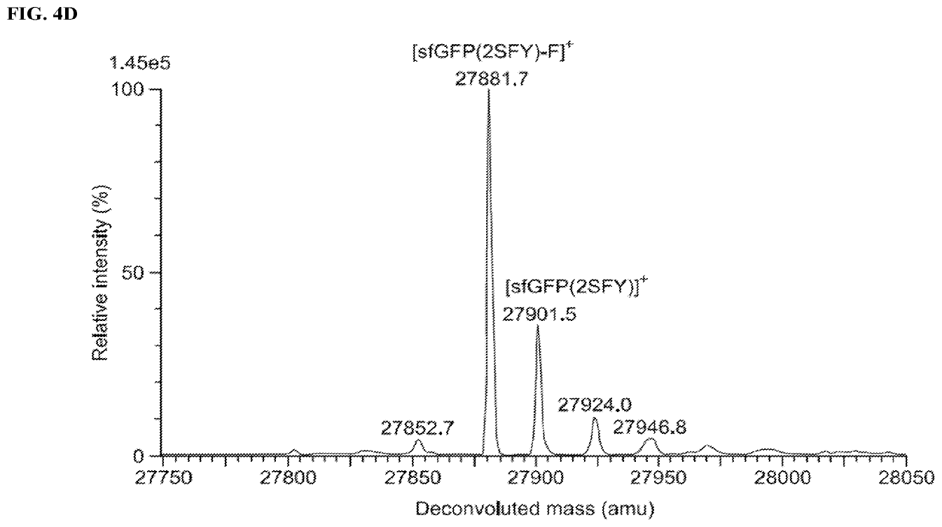

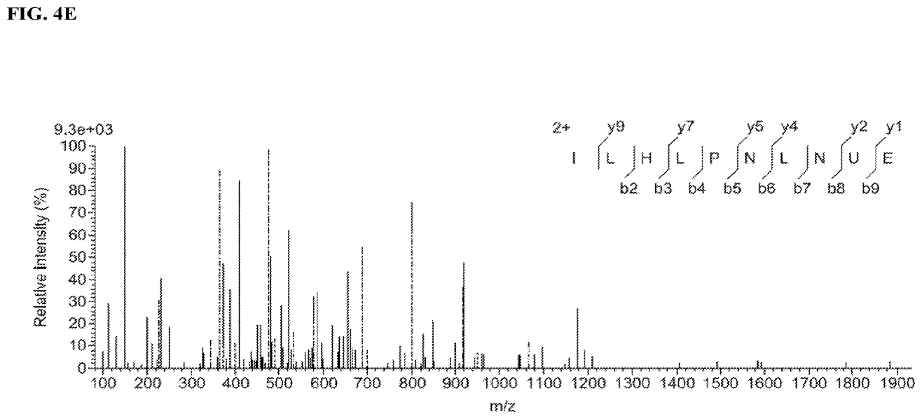

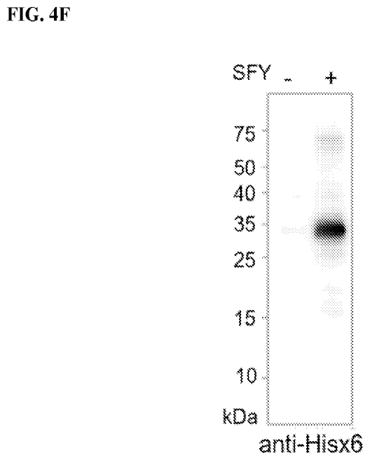

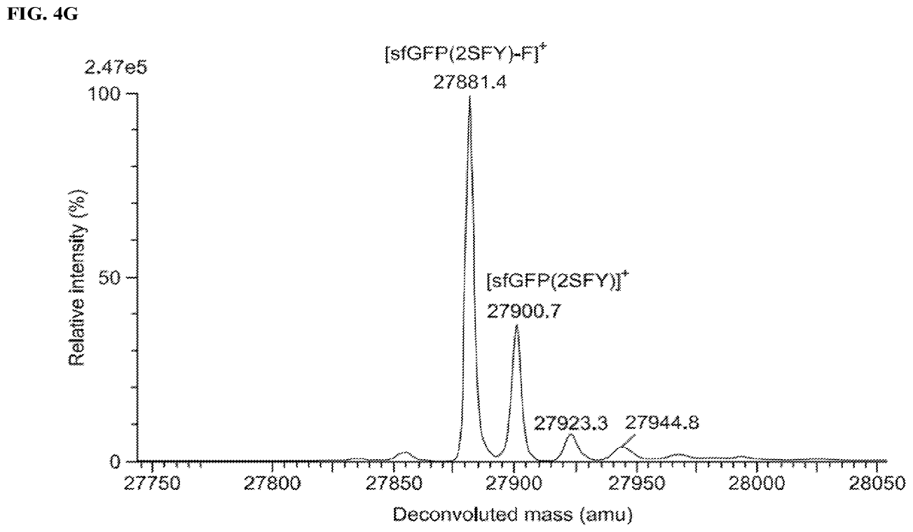

To introduce the identified sulfonyl fluoride group into proteins, we designed and synthesized the unnatural amino acid (Uaa) o-sulfonyl fluoride-O-methyltyrosine (SFY) (FIG. 4A). The sulfonyl fluoride group was placed at the meta rather than the para position of the phenyl ring, because we previously found a functional group introduced at the meta position has larger reaction area than at the para position possibly due to the rotation of the phenyl ring. (Ref. 21). The methoxy group was included to reduce the reactivity of sulfonyl fluoride, which would avoid potential cytotoxicity and increase reaction specificity. SFY contains sulfonyl chloride, which is more reactive than fluorosulfate in the previously genetically encoded latent bioreactive Uaa fluorosulfate-L-tyrosine (FSY).

We next evolved a mutant pyrrolysyl-tRNA synthetase (PylRS) specific for SFY to genetically incorporate it into proteins. A PylRS mutant library was generated by mutating residues Ala302, Leu305, Tyr306, Leu309, Ile322, Asn346, Cys348, Tyr384, Val401, and Trp417 of the Methanosarcina mazei PylRS using the small-intelligent mutagenesis approach, and subjected to selection as described. (Refs. 33-35). A hit showing SFY-dependent phenotype was identified, which contained the following mutations (306L/309A/346A/348M/417T) and was named as MmSFYRS (FIG. 4B).

To evaluate the incorporation specificity of SFY into proteins in E. coli, we expressed the superfold green fluorescent protein (sfGFP) gene containing a TAG codon at position 2 (sfGFP-2TAG) with the tRNA^{Pyl}/MmSFYRS pair in E. coli. When 1 mM SFY was added in growth media, full-length sfGFP(2SFY) was significantly produced (FIG. 4C). The purified sfGFP(2SFY) was analyzed with ESI-TOF MS (FIG. 4D). A peak observed at 27901.5 corresponds to intact sfGFP containing SFY at site 2 (expected 27900.9 Da). Another peak measured at 27881.7 Da corresponds to sfGFP(2SFY) lacking F (expected 27881.9 Da), suggesting some F elimination during MS measurement. (Ref. 25). No peaks corresponding to sfGFP containing other amino acids at site 2 were observed. We also incorporated SFY at position 24 of the Z protein in E. coli, and analyzed the purified Z protein with tandem MS (FIG. 4E). A series of b and y ions unambiguously indicate that SFY was incorporated at site 24 of the Z protein. These results indicate that the tRNA^{Pyl}/MmSFYRS pair incorporated SFY into proteins with high specificity in E. coli.

We further transplanted the mutations of MmSFYRS into Methanomethylophilus alvus PylRS to generate MaSFYRS, as transplanting mutations from Methanosarcina barkeri PylRS mutant into M, alvus PylRS has been shown to increase the Uaa incorporation efficiency. (Ref. 27). Expression of sfGFP-2TAG gene together with the Ma-tRNA^{Pyl}/MaSFYRS pair in E. coli also showed SFY dependent production of full-length sfGFP (FIG. 4F). ESI-TOF MS analysis of the purified sfGFP(2SFY) yielded similar peaks as observed in FIG. 4D, confirming that Ma-tRNA^{Pyl}/MaSFYRS also had high specificity in incorporating SFY into proteins in E. coli (FIG. 4G). To compare incorporation efficiency, we then incorporated SFY into GFP at permissive sites 2, 40, and 182, respectively, using the Ma-tRNA^{Pyl}/MaSFYRS pair or the Mm-RNA^{Pyl}/MmSFYRS. Through quantifying the fluorescence intensity of the expressed GFP in E. coli cells, we found that the Ma-tRNA^{Pyl}/MaSFYRS increased the SFY incorporation efficiency in E. coli over the Mm-tRNA^{Pyl}/MmSFYRS more than 10 fold, reaching a yield of over 50 mg/L GFP (FIG. S5). The Ma-tRNA^{Pyl}/MaSFYRS pair was thus used in subsequent experiments.

### Siglec-7v(SFY) Enables Cross-Linking of Sialoglycan In Vitro and on Mammalian Cell Surface

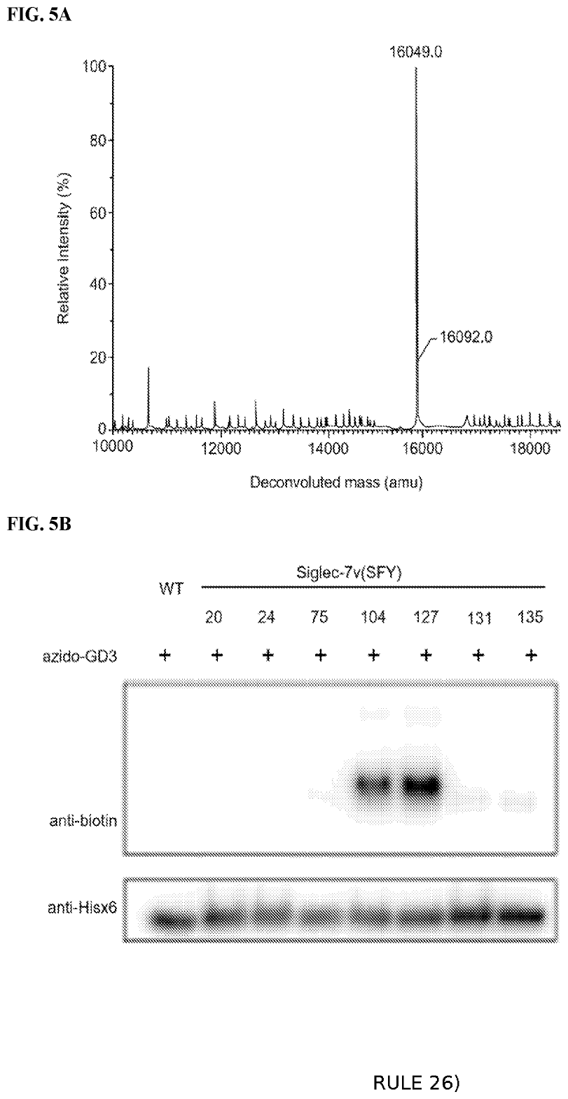

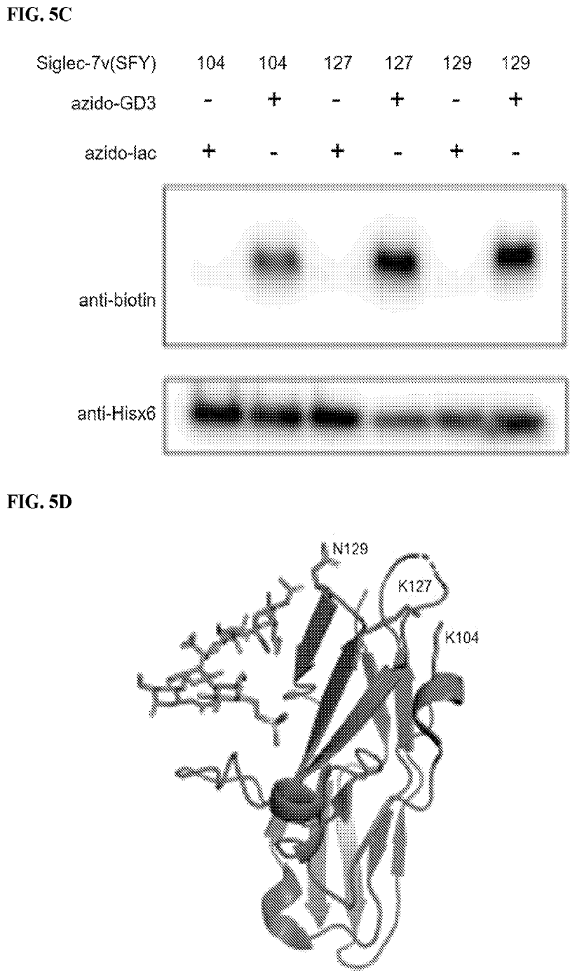

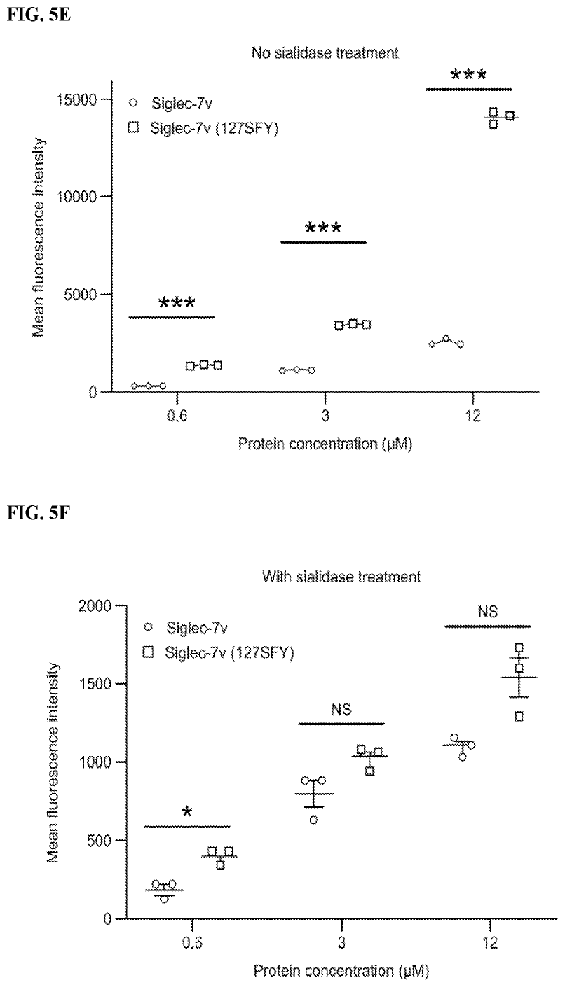

To prepare SFY incorporated Siglec-7v proteins, we expressed Siglec-7v(104TAG) gene with the Ma-tRNA^{Pyl}/MaSFYRS in E. coli in the presence of 1 mM of SFY. The expressed Siglec-7v(104SFY) protein was purified and refolded similarly as WT Siglec-7v. The Siglec-7v(104SFY) protein was produced with a yield of 5 mg/L, and the WT Siglec-7v yielded 20 mg/L. The intact mass of the purified Siglec-7v(104SFY) was analyzed with ESI-TOF MS (FIG. 5A). A major peak was measured at 16049.0 Da, corresponding to the intact Siglec-7v(104SFY) protein lacking F (expected 16049.6 Da), suggesting loss of F during MS measurement.

We then determined if SFY could enable Siglec-7v to cross-link the bound glycan ligand. Inspired by NHSF-mediated cross-linking of azido-GD3 through Lys on Siglec-7v protein, we incorporated SFY into individual Lys sites of Siglec-7v, including sites 20, 24, 75, 104, 127, 131, and 135. Each SFY incorporated Siglec-7v mutant was incubated with 2 mM azido-GD3 for 1 h at room temperature, followed with click labeling of alkyne-biotin and Western blot analysis using streptavidin-HRP (FIG. 5B). Cross-linking of azido-GD3 was detected for Siglec-7v(104SFY) and Siglec-7v(127SFY). Since site 127 showed positive cross-linking of azido-GD3 in both NHSF- and SFY-mediated cross-linking, we also tested its two adjacent sites 129 and 130. Indeed. Siglec-7v(129SFY) also cross-linked azido-GD3 (FIG. 5C). When the control ligand azido-lac was instead used in binding, these three Siglec-7v mutants (104SFY. 127SFY, and 129SFY) all showed a background band which also appeared in WT Siglec-7v. Moreover, addition of 3′-sialyllactose, another control ligand lacking the very terminal Neu5 Ac, did not reduce the crosslinking of siglec-7v(127SFY) with azido-GD3. On the basis of the crystal structure of Siglec-7 in complex with α(2.8)-disialylated ganglioside GT1b, sites 127 and 129 are both in close proximity to the terminal sialic acid Neu5 Ac of the bound GT1b, accounting for the observed SFY cross-linking of azido-GD3. (Ref. 32). On the contrary, site 104 is located at the opposite side of the GT1b binding site, which seemingly has no contact with the glycan ligand. However, a recent in silico analysis and mutagenesis study suggests a new sialic acid-binding region of Siglec-7 containing Arg67. (Ref. 36). Siglec-7v(104SFY) cross-linking of azido-GD3 is consistent with this proposal. Together, these results indicate that Siglec-7v mutants with SFY incorporated at the appropriate sites were able to cross-link the bound glycan in a proximity based manner.

We further explored if SFY incorporated Siglec-7v could cross-link sialoglycan on mammalian cell surface. SK-MEL-5 is a human melanoma cell line with a high level of sialylation on cell surface. (Ref. 37). We incubated SK-MEL-5 cells with different concentrations of WT Siglec-7v or Siglec-7v(127SFY), followed with washing. Siglec-7v proteins bound to cell surface were stained with a fluorescently labeled antibody specific for the His×6 tag appended at the C-terminus of Siglec-7v, and quantified with flow cytometry. Remarkably, cells incubated with Siglec-7v(127SFY) showed higher mean fluorescence intensity (MFI) over those incubated with WT Siglec-7v in all protein concentrations tested (FIG. 5E). The cell MFI difference reached 5.6-fold when 12 μM of protein was used. This binding difference indicated that WT Siglec-7v which bound in non-covalent mode would dissociate from cell surface sialoglycan during washing, whereas Siglec-7v(127SFY) could remain bound due to the covalent cross-linking. To confirm that the strong fluorescence signal of Siglec-7v(127SFY) was mainly due to its cross-linking with sialoglycan on cell surface, we pre-treated the cells with sialidase to remove the terminal sialic acids on cell surface, which Siglec-7v proteins prefer binding with. (Ref. 38). The pretreated SK-MEL-5 cells were then incubated with WT Siglec-7v or Siglec-7v(127SFY), washed, and stained for quantification of bound Siglec-7v. No significant MFI difference was measured between cells incubated with WT Siglec-7v or Siglec-7v (127SFY) (FIG. 5F). Therefore, after sialidase treatment. Siglec-7v(127SFY) had similar binding on cell surface as WT Siglec-7v. Taken together, these results demonstrate that Siglec-7v(127SFY) was able to cross-link the sialoglycan on mammalian cell surface.

### Siglec-7v(127SFY) Enhances NK Cell Killing of Cancer Cells

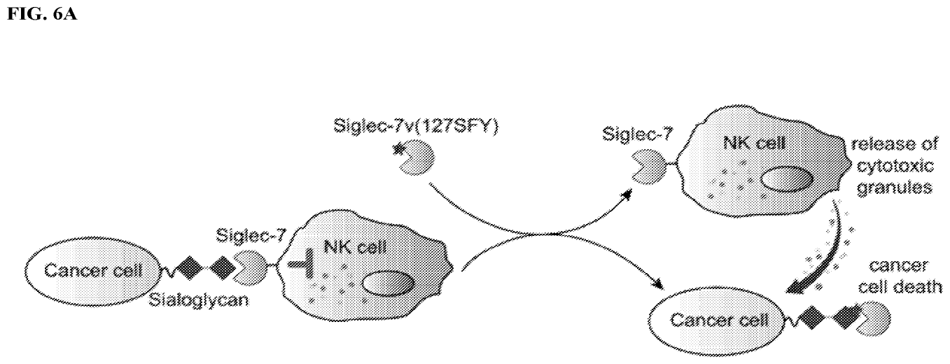

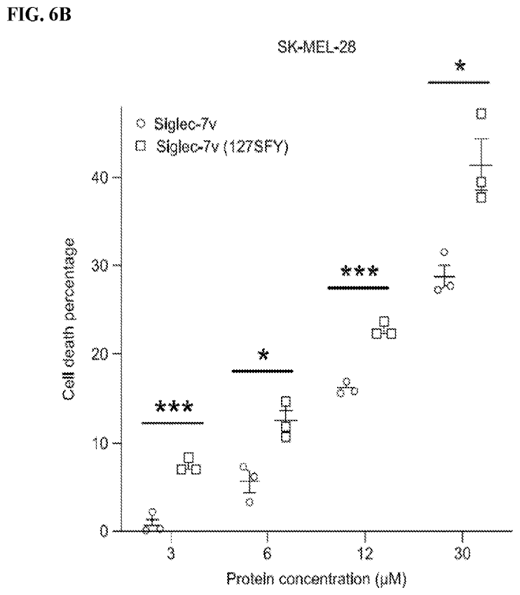

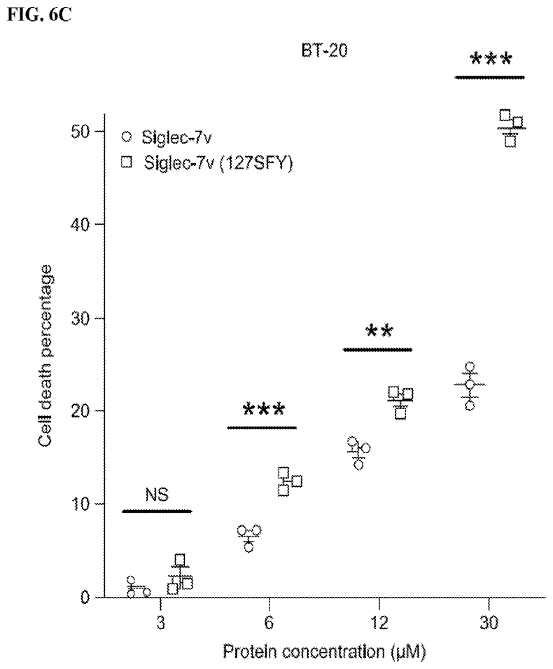

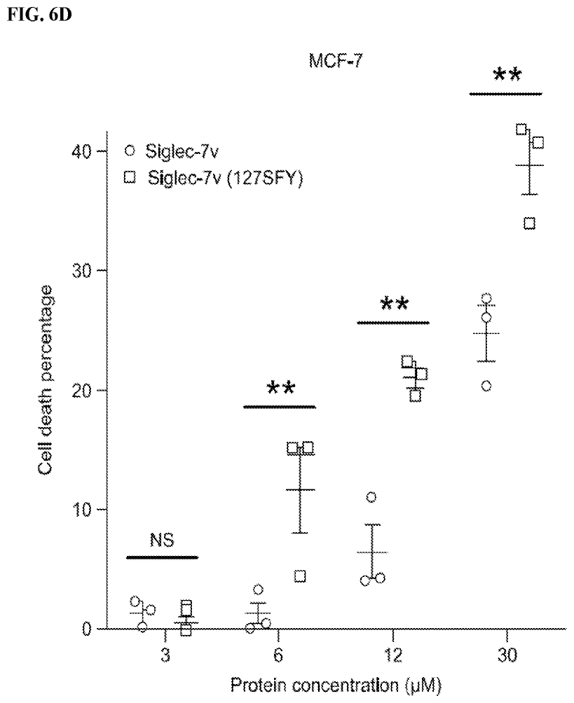

Many tumors upregulate cell surface sialic acids, which bind with Siglec-7 on human NK cells, inhibiting NK cell cytotoxicity and evading immune-surveillance. Since Siglec-7v(127SFY) could irreversibly cross-link with cell surface sialoglycan, we reasoned that it would competitively block the interaction of tumor cell surface sialoglycan with Siglec-7 of NK cells, thus enhancing NK cell killing of tumor cells (FIG. 6A).

To test this hypothesis, we incubated Siglec-7v(127SFY) with three hypersialylated human cancer cell lines, SK-MEL-28 (melanoma), BT-20 (breast carcinoma), and MCF-7 (breast adenocarcinoma), respectively for 2 h to allow binding and cross-linking, using WT Siglec-7v as the control. (Ref. 39). The cells were washed and then subjected to incubation with human NK-92 cells. NK-92 is a cytotoxic human NK cell line that is currently in clinical trials for cancer treatment. (Ref. 40). Cancer cell viability was evaluated with propidium iodide staining and quantified with flow cytometry. The percent of cancer cells killed by NK-92 cells was calculated (FIGS. 6B-6D). For all three cancer cell lines tested, Siglec-7v(127SFY) enhanced NK-92 killing of cancer cells over WT Siglec-7v in the same concentration. The percent of dead cancer cells increased with the concentration of Siglec-7v applied. Siglec-7v(127SFY) was thus more potent than WT Siglec-7v, requiring the latter in higher concentration to reach similar level of cancer cell killing. These results indicate that covalent binding of Siglec-7v(127SFY) with sialoglycan on cancer cell surface more effectively enhanced NK killing of cancer cells than the WT Siglec-7v.

A major advantage of genetically encoding SFY is the ability to introduce the sulfonyl fluoride into the Siglec-7v protein site specifically. Although sulfonyl fluoride could be installed on Siglec-7v through pretreating Siglec-7v with NHSF, this approach resulted in the installation of suflonyl fluoride at multiple Lys sites nonselectively. Consequently, NHSF-pretreated Siglec-7v failed to bind with azido-GD3 in vitro and with sialoglycan on BT20 or SK-MEL-28 cells. As expected, NHSF-pretreated Siglec-7v also had no effect in enhancing NK cell killing of cancer cells. These results indicate the importance of site specificity enabled by genetic encoding.

### DISCUSSION

We identified sulfonyl fluoride to cross-link glycan via proximity-enabled reactivity by applying plant-and-cast cross-linkers onto protein-glycan complex. A novel bioreative Uaa SFY bearing sulfonyl fluoride was then designed and genetically incorporated into proteins via genetic code expansion. SFY-incorporated Siglec-7v specifically cross-linked its sialoglycan ligand in vitro and on cancer cell surface. Moreover, through covalently cloaking sialoglyan on cancer cell surface. Siglec-7v(SFY) significantly enhanced NK cell killing of cancer cells over the noncovalent WT Siglec-7v.

Protein-glycan interactions are noncovalent in nature. Through developing the GECX-sugar technology, here we changed this paradigm and enabled the site-specific introduction of covalent linkages into interacting protein-glycan for the first time. The latent bioreactive Uaa SFY is genetically encoded into the protein to achieve residue specificity for the covalent linkage. SFY remains stable inside cells and in the protein. The reaction of SFY with glycan is enabled by the close proximity of SFY side chain to the glycan hydroxyl group when protein binds to glycan. Therefore, through strategically placing SFY into different sites of the protein, monosaccharide selectivity for the bound glycan can also be achieved for the covalent linkage. This site-specificity for both protein and glycan of GECX-sugar will enable the precise engineering of covalent linkages to cross-link protein to the interacting glycan. Such irreversible cross-linking fundamentally overcomes the general low affinity of glycan toward protein. Similar to how covalent cross-linking of proteins by GECX has enabled the identification of weak protein-protein interactions. GECX-sugar should provide a new route to the identification of the weak and transient protein-glycan interactions. (Ref. 11). In addition, in contrast and complementary to metabolic pathway engineering which modifies the glycan. GECX-sugar is able to covalently target endogenous glycans and thus suitable for in vivo studies and therapeutic applications. (Refs. 41-42). Cross-linking of protein to the unmodified glycan converts the binding protein into an irreversible inhibitor for the native protein-glycan interaction, which can be exploited for glycan-based diagnostic and therapeutic applications, such as enhancing NK cell killing of cancer cells demonstrated here. In essence. GECX-sugar is able to transform a glycan binding protein into a non-antibody binder for specific glycan with high affinity.

Siglec-7v(SFY) covalently cross-linked to its sialoglycan ligand specifically. Based on the SuFEx reactivity of sulfonyl fluoride and SFY incorporation site in Siglec-7v. SFY should have reacted with the hydroxyl group of sialic acid. As all monosaccharides contain the hydroxyl group, we expect that SFY can be incorporated into other glycan binding proteins to covalently target various glycans, which will be verified experimentally in the future. Siglec-7v(SFY) significantly increased NK killing of cancer cells in vitro, but its anti-tumor effect in vivo awaits demonstration. Since covalent PD-1 containing SuFEx bioreactive SFY shows dramatically enhanced anti-tumor effect than the noncovalent WT PD-1 in multiple xenograft mouse models, primarily due to decoupling of the pharmacodynamics and pharmacokinetics via the covalent mechanism, we expect that the difference in enhancing NK killing between Siglec-7v(SFY) and WT Siglec-7v would be similarly more drastic in vivo than in vitro. (Ref. 12).

In summary, GECX-sugar enables site-specific introduction of covalent linkages between proteins and glycans, providing a solution to the long-standing challenge of low affinity and weak interaction. GECX-sugar will thus advance the basic study of glycobiology and inspire new avenues for protein diagnostics and therapeutics via effectively targeting glycan.

### Experimental Procedures

### Molecular Cloning

Primers were synthesized by Integrated DNA Technologies (IDT), and all plasmids were sequenced by GENEWIZ. All reagents were obtained from New England Biolabs.

Siglec-7v
(SEQ ID NO: 1)
MQKSNRKDYSLTMQSSVTVQEGMSVHVRCSFSYPVDSQTDSDPVHGYWF
RAGNDISWKAPVATNNPAWAVQEETRDRFHLLGDPQTKNCTLSIRDARM
SDAGRYFFRMEKGNIKWNYKYDQLSVNVTALTHHHHHHH

Positions K20, K24, K75, K104, K127, N129, I130, K131, K135 are in bold and underlined.

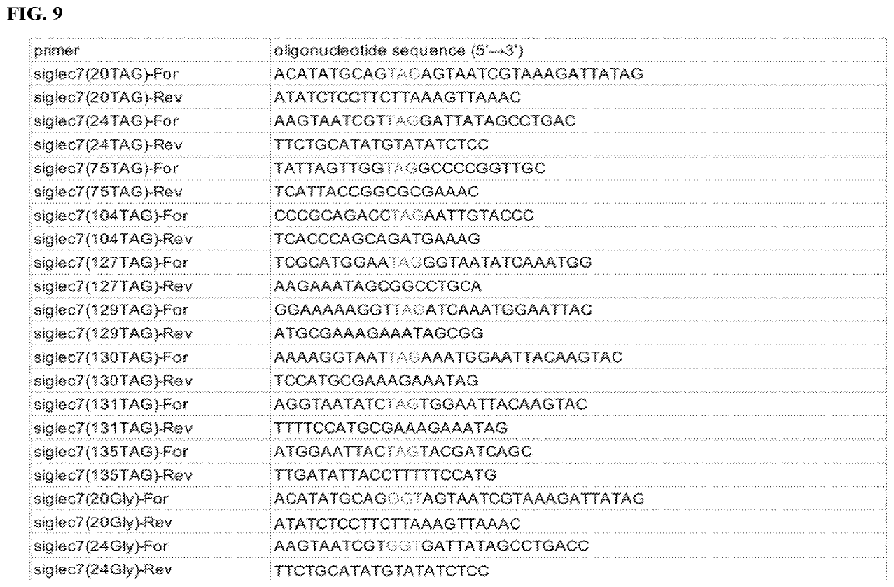

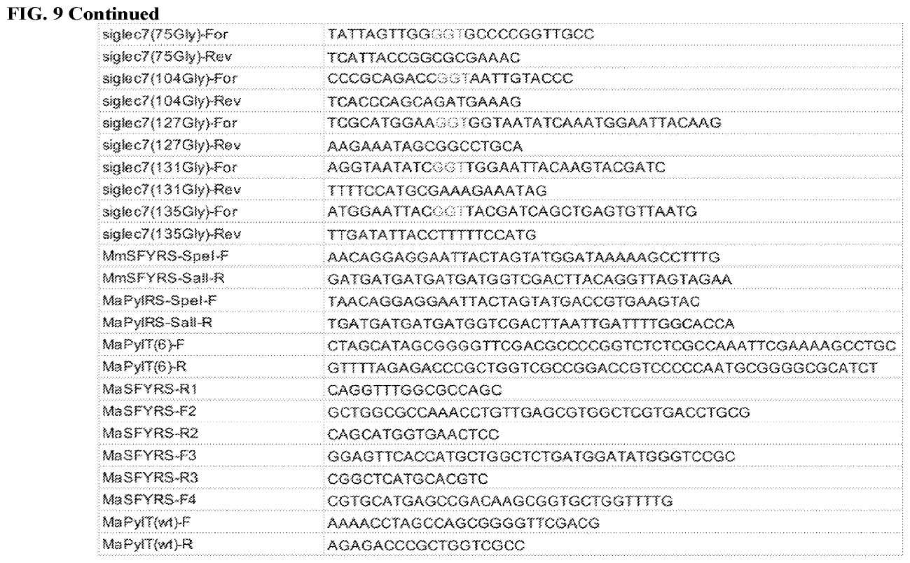

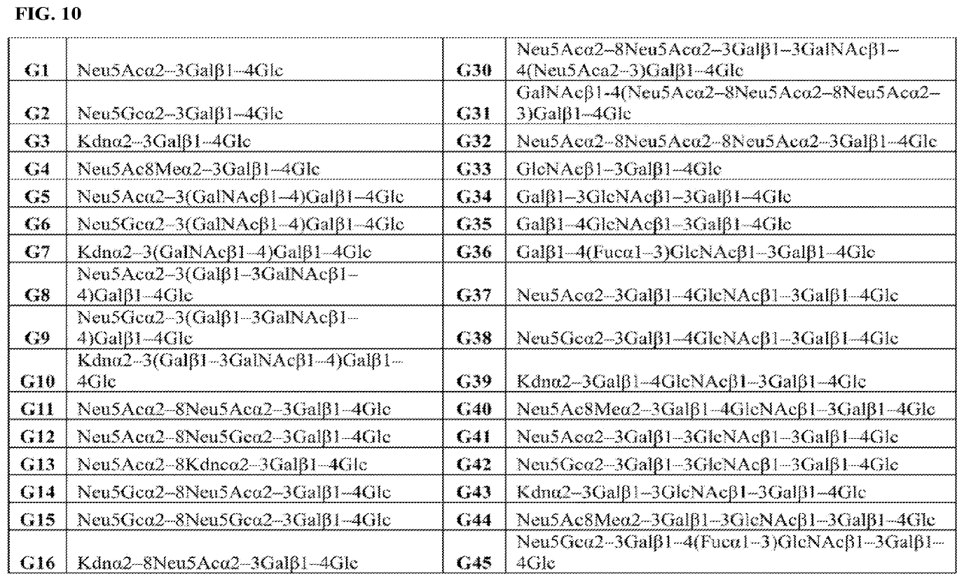

The Siglec-7v gene was synthesized by IDT. Residue 20, 24, 75, 104, 127, 129, 130, 131 or 135 of Siglec-7v was mutated to an amber stop codon TAG, respectively, via site-directed mutagenesis using primers in FIG. 9.

sfGFP (2SFY)
(SEQ ID NO: 2)
MUKGEELFTGVVPILVELDGDVNGHKFSVRGEGEGDATNGKLTLKFICT
TGKLPVPWPTLVTTLTYGVQCFSRYPDHMKRHDFFKSAMPEGYVQERTI
SFKDDGTYKTRAEVKFEGDTLVNRIELKGIDFKEDGNILGHKLEYNFNS
HNVYITADKQKNGIKANFKIRHNVEDGSVQLADHYQQNTPIGDGPVLLP
DNHYLSTQSVLSKDPNEKRDHMVLLEFVTAAGITHGMDELYKGSHHHHH
H

Bold Underlined: amber codon TAG at 2nd position

pEvol-MmSFYRS, pEvol-MmSFYRS plasmid was generated by introducing the MmSFYRS encoding gene into pEvol vector via homologous recombination. Briefly, the SFYRS gene was amplified with primers MmSFYRS-SpeI-F and MmSFYRS-SalI-R, purified, and ligated into pEvol vector (linearized with SpeI and SalI) with Exnase™ II.

pEvol-MaPylRS-wt. According to gene alignment, the active sites of Methanosarcina mazei PylRS (MmPylRS) and Methanomethylophilus alvus PylRS (MaPylRS) are highly conserved. However, MaPylRS and its derivatives usually present better solubility than those synthetases originated from MmPylRS, which may lead to higher incorporation efficiency. In order to enhance the incorporation efficiency of SFY, we decided to examine the incorporation of SFY using the Ma-tRNA^{Pyl}/PylRS pair. To achieve this goal, a pEvol-MaPylRS plasmid encoding an orthogonal pair of wt-MapylRS and evolved MaPylT was first constructed. Briefly, the wild-type MaPylRS gene (Supp Ref. 1) was chemically synthesized, amplified with MaSFYRS-SpeI-F/MaSFYRS-SalI-R primers, and introduced into the pEvol vector via homologous recombination. Then an evolved Ma-pyrrolysyl-tRNA gene MaPylT(6) (Supp Ref 2) was introduced into pEvol vector via site-directed mutagenesis with MaPylT(6)-F/R primers. The resultant plasmid was named as pEvol-MaPylRS-wt and used as the template to generate pEvol-MaSFYRS.

pEvol-MaSFYRS. Mutations carried by MmSFYRS were directly transplanted into MaPylRS via PCR-amplification with primers (MaSFYRS-R1, —F2, —R2, —F3, —R3,) and then ligated into the pEvol vector via multiple-fragment homologous recombination. To further improve the incorporation efficiency of SFY, the evolved MaPylT(6) was swapped with the wild-type MaPylT by using site-directed mutagenesis with MaPylT(wt)-F/R primers to afford the pEvol-MaSFYRS plasmid. As shown in Figure S5, the WT-MapylT afforded much higher incorporation efficiency than the evolved MaPylT(6) (indicated as MaPylT-mut in the figure).

### Chemo-Enzymatic Synthesis of Azido-GD3 and Azido-Lac

The scheme for chemo-enzymatic synthesis of azido-GD3 and azido-lac was shown in FIG. 7. Azidolactose (compound 1) was chemically synthesized as described α-2,3 sialic acid transferase was used for the addition of sialic acid to get the azido-trisaccharide, compound 3. Compound 5 was synthesized via enzymatic catalysis with compound 3, compound 4, and pyruvate in the presence of aldolase, CMP-sialic acid synthetase and α-2,8 sialic acid transferase. The final product azido-GD3 was purified using HPLC and characterized with ESI-MS. [M+H], [M+Na] peaks of azido-GD3 were observed.

### Chemical Synthesis

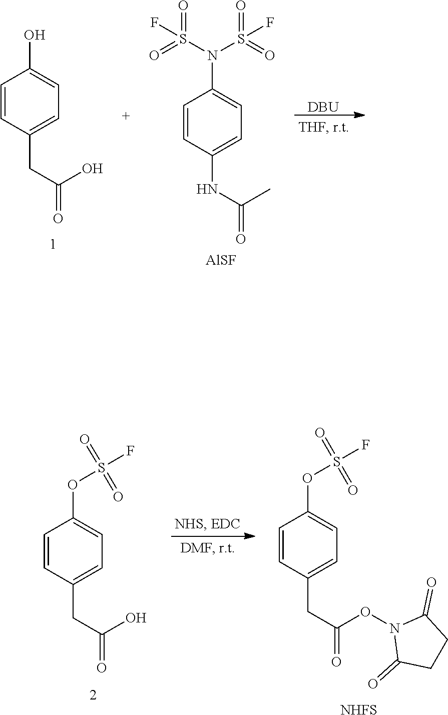

Synthesis of 2-(4-((fluorosulfonyl)oxy)phenyl)acetic acid (2). 2-(4-hydroxyphenyl)acetic acid (1) was converted to fluorosulfate using [4-(acetylamino)phenyl]imidodisulfuryl difluoride (AISF). (Supp Ref. 3) 1.5 g compound 1 (1.5 g, 9.9 mmol) and AISF (3.4 g, 10.8 mmol) was dissolved in 50 mL anhydrous THF. Then 1,8-Diazabicyclo[5.4.0]undec-7-ene (DBU, 3.2 g, 22 mmol) was added dropwise at room temperature (r.t.). The mixture was stirred at r.t, for 10 min. Then 200 mL EtOAc was added to dilute the reaction mixture and the organic phrase was washed sequentially by H₂O (100 mL) and brine (100 mL). The organic phase was dried over anhydrous Na₂SO₄ and evaporated under reduced pressure to give the crude product, which was then purified by column chromatography (silica gel, DCM:MeOH=50:1) to give a white solid (1.2 g, 53%).

Synthesis of 2,5-dioxopyrrolidin-1-yl 2-(4-((fluorosulfonyl)oxy)-phenyl)acetate (NHFS). To a stirred solution of compound 2 (500 mg, 2.1 mmol) and N-hydroxysuccinimide (NHS, 358 mg, 3.1 mmol) in 4 mL anhydrous DMF was added N-(3-Dimethylaminopropyl)-N′-ethylcarbodiimide hydrochloride (EDC·HCl, 611 mg, 3.2 mmol). The mixture was stirred at r.t. for 24 h. Then the reaction was quenched with the addition of H₂O (30 mL) and the mixture was extracted with EtOAc (2×30 mL). The combined organic phase was dried over anhydrous Na₂SO₄ and evaporated under reduced pressure to give the crude product, which was purified by column chromatography (silica gel, DCM:EtOAc=50:1) to give a white solid (452 mg, 65%). ¹H NMR (CDCl₃): δ 7.46 (d, J=8.8 Hz, 2H), 7.34 (d, J=8.8 Hz, 2H), 3.98 (s, 2H), 2.84 (s, 4H). ¹³C NMR (CDCl₃): δ 169.0, 166.2, 149.6, 132.4, 131.5, 121.5, 37.0, 25.7. NHFS itself have poor signal during mass spectrum analysis. NHFS was converted to compound 4 for mass analysis. Briefly, 20 mM compound NHFS, 20 mM tert-butyl (3-aminopropyl)carbamate (3) and 20 mM NaOH was incubated in H₂O at r.t, for 2 h. Then the solution was subject to mass spectrum analysis. HRMS calcd for C₁₆H₂₃FN₂Na₂O₆S [M+Na]⁺413.1153, found: 413.1158.

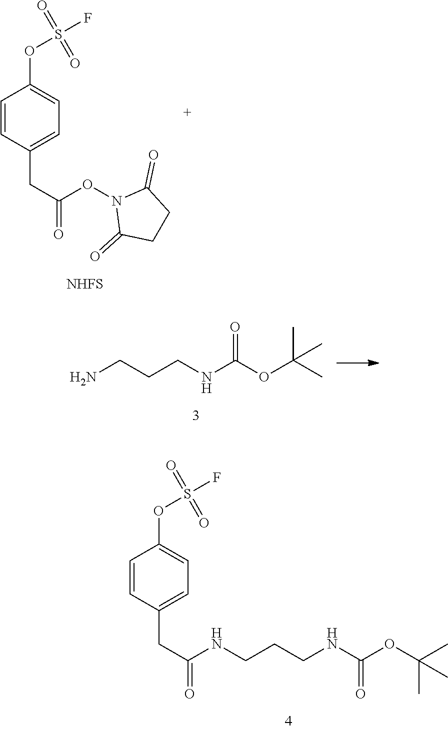

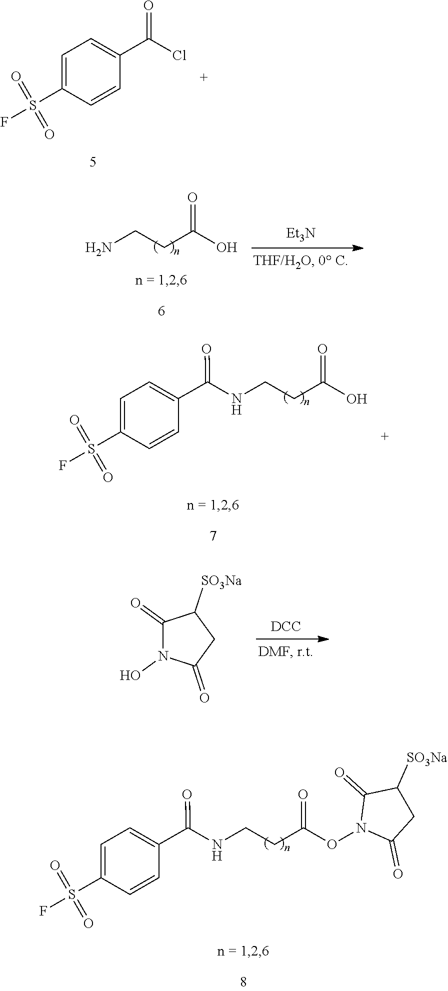

### General Synthetic Procedure for Compound 8.

Compound 5 was synthesized from 4-(fluorosulfonyl)benzoic acid using SOCl₂ according to literature procedure. (Supp Ref 4). Synthesis of compound 7. To a stirred solution of compound 6 (2.5 mmol) and triethylamine (Et₃N, 5.0 mmol) in H₂O (2 mL) was added dropwise compound 5 (2.5 mmol) in THF (4 mL) at 0° C. The mixture was allowed to warm to room temperature and was stirred for 2 h. Then the reaction was quenched with the addition of H₂O (25 mL) and the mixture was extracted with EtOAc (2×25 mL). The combined organic phase was dried over anhydrous Na₂SO₄ and evaporated under reduced pressure to give the crude product, which was purified by column chromatography (silica gel, DCM:MeOH=20:1) to give a white solid (about 70%).

Synthesis of compound 8. Compound 7 (0.72 mmol), N-Hydroxysulfosuccinimide sodium salt (0.72 mmol) and N,N′-Dicyclohexylcarbodiimide (DCC, 0.72 mmol) was dissolved 1.5 mL anhydrous DMF. The mixture was stirred at r.t, for 24 h under N₂. A white precipitate was formed during the reaction and was removed by filtration. 20 mL diethyl ether was added to the filtrate, and a white precipitate was formed and collected by centrifuge (10 min, 3,000 rpm). The white precipitate was redissolved in 4 mL MeOH and 20 mL diethyl ether was added, and a white precipitate was formed and collected by centrifuge (10 min, 3,000 rpm). The white precipitate was further purified by preparation HPLC (C18 column) using H₂O/ACN (0.05% TFA) as mobile phrase (˜65%).

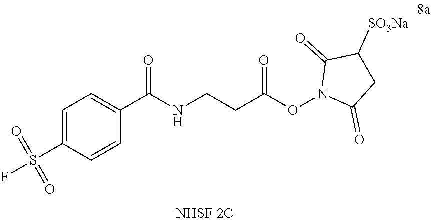

Sodium 1-((3-(4-(fluorosulfonyl)benzamido)propanoyl)oxy)-2,5-dioxopyrrolidine-3-sulfonate (8a, NHSF 2C). ¹H NMR (DMSO): δ 9.08 (t, J=5.6 Hz, 1H), 8.26 (d, J=8.4 Hz, 2H), 8.15 (d, J=8.4 Hz, 2H), 3.94 (d, J=8.0 Hz, 1H), 3.63-3.58 (m, 2H), 3.14-3.07 (m, 1H), 3.02 (t, J=6.4 Hz, 2H), 2.85 (dd, J=16.0 Hz, J=2.4 Hz, 1H). ¹³C NMR (DMSO): δ 168.7, 165.2, 164.7, 141.2, 133.6 (d, J=23 Hz, C—F), 129.1, 128.7, 56.3, 35.2, 31.0, 30.2. HRMS calcd for C₁₄H₁₂FN₂Na₂O₁₀S₂[M+Na]⁺496.9707, found: 496.9716.

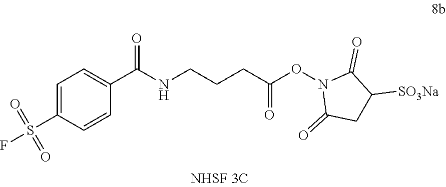

Sodium 1-((4-(4-(fluorosulfonyl)benzamido)butanoyl)oxy)-2,5-dioxopyrrolidine-3-sulfonate. (8b, NHSF 3C). ¹H NMR (DMSO): δ 8.94 (t, J=5.6 Hz, 1H), 8.24 (d, J=8.4 Hz, 2H), 8.17 (d, J=8.4 Hz, 2H), 3.95 (d, J=8.8 Hz, 1H), 3.40-3.35 (m, 2H), 3.20-2.83 (m, 2H), 2.78 (t, J=7.6 Hz, 2H), 1.94-1.87 (m, 2H), ¹³C NMR (DMSO): δ 168.8, 165.4, 164.2, 141.7, 133.3 (d, J=24 Hz, C—F), 129.0, 128.6, 56.3, 38.6, 31.0, 27.9, 24.0. HRMS calcd for C₁₅H₁₄FN₂Na₂O₁₀S₂[M+Na]⁺510.9864, found: 510.9851.

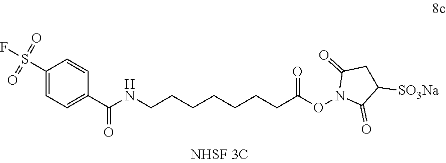

sodium 1-((8-(4-(fluorosulfonyl)benzamido)octanoyl)oxy)-2,5-dioxopyrrolidine-3-sulfonate (8c). ¹H NMR (DMSO): δ 8.84 (t, J=5.6 Hz, 1H), 8.24 (d, J=8.4 Hz, 2H), 8.15 (d, J=8.4 Hz, 2H), 3.94 (d, J=8.4 Hz, 1H), 3.30-3.26 (m, 2H), 3.15-2.82 (m, 2H), 2.65 (t, J=7.6 Hz, 2H), 1.64-1.52 (m, 4H), 1.38-1.30 (m, 6H). ¹³C NMR (DMSO): δ 168.8, 165.3, 164.6, 141.5, 133.4 (d, J=24 Hz, C—F), 129.2, 128.6, 56.3, 31.0, 30.2, 28.8, 28.2, 28.0, 26.3, 24.3. HRMS calcd for C₁₉H₂₃FN₂NaO₁₀S₂[M+H]⁺ 545.0670, found: 545.0660.

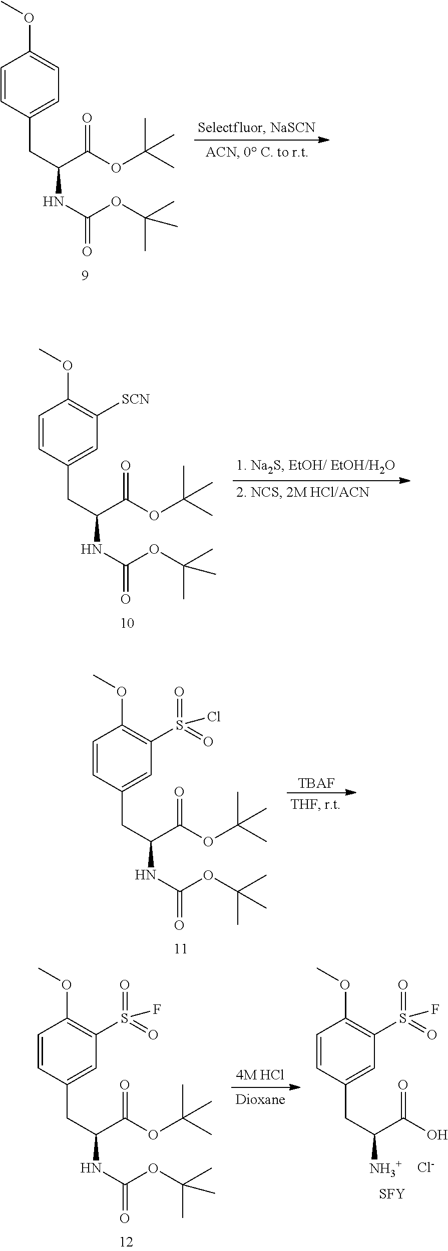

Synthesis of tert-butyl (S)-2-((tert-butoxycarbonyl)amino)-3-(4-methoxy-3-thiocyanatophenyl)propanoate (10). To a stirred solution of Selectfluor (2.4 g, 6.8 mmol) and NaSCN (550 mg, 6.8 mmol) in ACN (20 mL) was added compound 9 (800 mg, 2.27 mmol) in ACN (5 mL) at 0° C. under N2. The reaction mixture was allowed to stir at r.t, for overnight. Then the solvent was removed under reduced pressure and the residue was dissolved in 25 mL EtOAc. The organic phrase was washed sequentially by H₂O (25 mL) and brine (25 mL). The organic phase was dried over anhydrous Na₂SO₄ and evaporated under reduced pressure to give the crude product, which was then purified by column chromatography (silica gel, Hexane:EtOAc=5:1) to give a yellow solid (637 mg, 69%). ¹H NMR (CDCl₃): δ 7.33 (d, J=2.0 Hz, ¹H), 7.18 (dd, J=8.4 Hz, J=2.0 Hz, 1H), 6.85 (d, J=8.4 Hz, 1H), 5.04 (d, J=8.0 Hz, 1H), 4.43-4.38 (m, 1H), 3.89 (s, 3H), 3.10-2.95 (m, 2H), 1.43 (d, 18H). ¹³C NMR (CDCl₃): δ 170.7, 155.6, 155.1, 131.8, 131.1, 130.7, 111.5, 110.4, 82.6, 80.0, 56.4, 54.9, 37.7, 28.4, 28.2. HRMS calcd for C₂₀H₂₈N₂NaO₅S [M+Na]⁺431.1611, found: 431.1627.

Synthesis of tert-butyl (S)-2-((tert-butoxycarbonyl)amino)-3-(3-(fluorosulfonyl)-4-methoxyphenyl)propanoate (12). To a solution of 10 (620 mg, 1.52 mmol) in EtOH (3.5 mL) was added Na₂S·9H₂O (730 mg, 3.0 mmol) in H₂O (12 mL) at 60° C. The reaction mixture was then heated at 85° C. for 2 h. The reaction mixture was then allowed to cool down to r.t., and 10 mL H₂O was added. The mixture was then adjusted to pH 6.5 with acetic acid and extracted with EtOAc (10 mL×3). The combined organic phase was dried over anhydrous Na₂SO₄ and evaporated under reduced pressure to give the crude thiol product as yellow solid, which was immediately used for the next step. To a stirred solution of N-chlorosuccinimide (0.65 g, 4.9 mmol) in 2M HCl (0.6 mL) and acetonitrile (2.5 mL) was added dropwise crude thiol in acetonitrile (1 mL) dropwise at 0° C. The mixture was stirred at 0° C. for another 30 min. The mixture was then diluted with EtOAc (10 mL), the organic phrase was washed sequentially by H₂O (10 mL) and brine (10 mL). The organic phase was dried over anhydrous Na₂SO₄ and evaporated under reduced pressure to give the crude sulfonyl chloride (compound 11) product as yellow oil (604 mg).

Half of the newly prepared crude sulfonyl chloride was used for the next step. To a stirred solution of Compound 11 (300 mg, 0.67 mmol) in anhydrous THF (2 mL) was added 1.3 mL 1M tetrabutylammonium fluoride (TBAF, 1.33 mmol) in THF. The mixture was stirred at r.t, for 1 h and the completion of reaction was monitored by mass spectrum. The mixture was then diluted with EtOAc (10 mL), the organic phrase was washed sequentially by H₂O (10 mL) and brine (10 mL). The organic phase was dried over anhydrous Na₂SO₄ and evaporated under reduced pressure to give the crude product, which was then purified by column chromatography (silica gel, DCM:EtOAc=25:1) to give compound 12 as white solid (76 mg. 24% for 3 steps). ¹H NMR (CDCl₃): δ 7.70 (d, J=2.0 Hz, 1H), 7.51 (dd, J=8.4 Hz, J=2.0 Hz, 1H), 7.03 (d. J=8.4 Hz, 1H), 5.07 (d, J=8.4 Hz, 1H), 4.43-4.38 (m, 1H), 3.98 (s, 3H), 3.16-2.98 (m, 2H), 1.42 (d, 18H). ¹³C NMR (CDCl₃): δ 170.3, 157.1, 155.1, 138.5, 132.1, 129.4, 121.1 (d, J=23 Hz, C—F), 112.9, 83.0, 80.1, 56.7, 54.8, 37.3, 28.4, 28.1. HRMS calcd for C₁₉H₂₈FNNaO₇S [M+Na]+456.1463, found: 456.1473.

Synthesis of (S)-1-carboxy-2-(3-(fluorosulfonyl)-4-methoxyphenyl)ethan-1-aminium (SFY). Compound 12 (76 mg. 0.18 mmol) was stirred in 4 M HCl in dioxane (0.5 mL) at r.t, for 24 h. Then 5 mL diethyl ether was added to the reaction mixture, and a white precipitate was formed and collected by centrifuge (10 min, 3,000 rpm). The white solid was further dried under reduced pressure to give SFY in HCl salt form (52 mg, 92%). ¹H NMR (D₂O): δ 7.87 (d. J=2.4 Hz, 1H), 7.74 (dd, J=8.8 Hz, J=2.4 Hz, 1H), 7.32 (d, J=8.8 Hz, 1H), 4.30 (t, J=6.8 Hz, 1H), 4.01 (s, 3H), 3.37-3.24 (m, 2H). ¹³C NMR (D₂O): δ 171.3, 157.5, 139.4, 131.6, 126.8, 119.5 (d, J=21 Hz, C—F), 114.3, 56.7, 54.0, 34.4. HRMS calcd for C₁₀H₁₃FNO₅S [M+H]⁺ 278.0493, found: 278.0510.

### Protein Expression and Purification

### Siglec-7v and Siglec-7v (127SFY)

For wildtype Siglec-7v expression, the plasmid pBAD-siglec-7v was transformed into E. coli BL21 (DE3). For the incorporation of SFY into siglec-7v, the plasmid pBAD-siglec-7v(TAG) and was co-transformed with pEVOL-SFYRS into E. coli BL21(DE3), and plated on LB agar plate supplemented with 100 μg/mL ampicillin and 34 μg/mL chloramphenicol. Several colonies were picked and inoculated in 50 mL 2×YT (5 g/L NaCl, 16 g/L Tryptone, 10 g/L Yeast extract). The cells were grown at 37° C., 220 rpm to an OD 0.5, the medium was then added with either 0.2% L-arabinose only or 0.2% L-arabinose plus 1 mM SFY, and the expression were carried out at 25° C., 220 rpm for 18-22 h. Cells were harvested at 3000 g. 4° C. for 10 min. For protein purification, cells were resuspended in lysis buffer (20 mM Tris-HCl pH 8.0, 200 mM NaCl, 20 mM immidazole) supplemented with EDTA free protease inhibitor cocktail, 1 μg/mL Dnase. The cells were opened by sonification, after which the cell lysis solution was centrifuged at 10,000 g at 4° C. for 15 min. The pellet was suspended in guanidine buffer (20 mM Tris-HCl pH 8.0, 200 mM NaCl, 6 M guanidine) and centrifuged at 10,000 g at 4° C. for 15 min. The supernatant was collected and incubated with 500 μL Ni-NTA affinity resin. The resin was washed with guanidine wash buffer (20 mM Tris-HCl pH 8.0, 200 mM NaCl, 20 mM immidazole, 6 M guanidine) for 3 times, and then the protein was eluted twice with 20 mM Tris-HCl pH 8.0, 200 mM NaCl, 300 mM immidazole, 6 M guanidine. The eluted protein was diluted into dialysis buffer (20 mM Tris-HCl pH 8.0, 200 mM NaCl) with 4 M guanidine to a final concentration of 0.1 mg/mL and dialyzed against dialysis buffer with 2 M or 0 M guanidine for 8 hr each at 4° C. The refolded protein was concentrated to 1 mg/mL for further use.

sfGFP(2SFY)

For the incorporation of SFY into sfGFP, the plasmid pBAD-sfGFP(2TAG) and was co-transformed with pEVOL-SFYRS into E. coli BL21(DE3), and plated on LB agar plate supplemented with 100 μg/mL ampicillin and 34 μg/mL chloramphenicol. Several colonies were picked and inoculated in 50 mL 2×YT (5 g/L NaCl, 16 g/L Tryptone, 10 g/L Yeast extract). The cells were grown at 37° C., 220 rpm to an OD 0.5, the medium was then added with either 0.2% L-arabinose only or 0.2% L-arabinose plus 1 mM SFY, and the expression were carried out at 18° C., 220 rpm for 18-22 h. Cells were harvested at 3000 g, 4° C. for 10 min. For protein purification, cells were resuspended in lysis buffer (20 mM Tris-HCl pH 8.0, 200 mM NaCl, 20 mM immidazole, EDTA free protease inhibitor cocktail, 1 μg/mL Dnase). The cells were opened by sonification, after which the cell lysis solution was centrifuged at 10,000 g at 4° C. for 15 min. The supernatant was collected and incubated with 500 μL Ni-NTA affinity resin. The resin was washed with wash buffer (20 mM Tris-HCl pH 8.0, 200 mM NaCl, 20 mM immidazole) for 3 times, and then the protein was eluted twice with 20 mM Tris-HCl pH 8.0, 200 mM NaCl, 300 mM immidazole.

### Z(24SFY)

For the incorporation of SFY into Z protein, the plasmid pBAD-Z(24TAG) and was co-transformed with pEVOL-SFYRS into E. coli BL21(DE3), and plated on LB agar plate supplemented with 100 μg/mL ampicillin and 34 μg/mL chloramphenicol. The Z protein expression and purification was same as described above.

### Glycan Microassay Analysis

Twenty μg/mL Siglec-7v was incubated with array at room temperature for 3 h with gentle shaking, then washed with TSMT buffer (20 mM Tris-HCl, pH 7.4, 150 mM NaCl, 2 mM CaCl₂), 2 mM MgCl₂ and 0.05% Tween-20) at room temperature for 3 times. Alexa Fluor 647 conjugated 6× His tag antibody was diluted and incubated with the array at room temperature for 2 h with gentle shaking. After 3 times wash with TSMT buffer, the array was scanned at 635 nm with GenePix 4000B. The microarray was analyzed according to the fluorescence intensity, and data was interpreted into a two-dimensional bar chart. The y-axis is the fluorescence intensity to reveal relative protein binding signals for each glycan.

### Small Molecule Mediated Siglec-7v Cross-Linking In Vitro

To test if small molecule cross-linker could cross-link Siglec-7v with azido-GD3 and azido-lac, 60 μM Siglec-7v was incubated with 2 mM azido-GD3 or azido-lac in PBS buffer, pH 7.4 at room temperature for 1 hr. The solution was treated with or without 0.3 mM NHSF or NHBr or NHFS or NHQM or HoQM at room temperature for 1 hr, respectively. The NHQM or HoQM was then illuminated with or without UV for 15 mins at wavelength 365 nm. After that, 200 μM alkyne-biotin, 0.05 mM CuSO4, 1 mM THPTA and 1 mM sodium ascorbate were added and the reaction mixture was incubated at room temperature in dark environment for 0.5 hours. Samples were then boiled at 95° C. for 5 mins and run Western blot against 6× His tag antibody or streptavidin-horseradish peroxidase (HRP).

Selection of SFY-specific synthetase (SFYRS)

DH10B cells (100 μL) harboring the pREP positive selection reporter was transformed with 122 ng of pBK-TK3 library via electroporation. The electroporated cells were subjected to selections by following procedures previously described. (Supp Refs 5-7). The pBK plasmids encoding the selected SFYRS gene were extracted by miniprep and separated from the reporter plasmids by DNA electrophoresis. The resulted pBK plasmids were analyzed by Sanger-sequencing.

### Siglec-7v(127SFY) Cross-Linking Sialoglycan on Mammalian Cell Surface

SK-MEL-5 cells were plated into 6-well plate and incubated for 24 h. 100 μL Vibrio cholerae sialidase (Sigma) or 100 μL PBS was added with 400 μL medium without FBS for 24 h treatment. To test if siglec-7v(127SFY) could cross-link sialoglycan on mammalian cell surface, different concentrations of siglec-7v or siglec-7v(127SFY) was incubated with SK-MEL-5 cells pre-treated with or without sialidase in PBS buffer, pH 7.4 at 37° C. 5% CO₂ incubator for 2 h. Cells was washed 3 times in PBS buffer and labeled with Alexa Fluor 488 conjugated 6× His tag monoclonal antibody at room temperature for 1 h. Cells were harvested for fluorescence-activated cell sorting (FACS) analysis.

### Siglec-7v(127SFY) Enhancing NK Cell Killing of Cancer Cells

Target cells were pre-labeled with CellTrace far red dye (Thermo Fisher Scientific) at room temperature for 10 min. Siglec-7v or siglec-7v(127SFY) of different concentrations was incubated with 5×10⁴ target cells in PBS buffer, pH 7.4 at 37° C. 5% CO₂ incubator for 2 h. Cells were washed 3 times in PBS buffer and subsequently incubated with 5×10⁵ NK cells in incubator for 4 h. Propidium iodide (10 μg/mL, Sigma) was added to each sample, and NK cell cytotoxicity was evaluated by fluorescence-activated cell sorting (FACS) analysis. Cells were acquired after electronic gating on CellTrace far red dye, and percentage of propidium iodide-positive cells was determined. Cell death percentage was calculated as experimental % death-control % death. Control % death was determined using the group without protein incubation.

### Mass Spectrometry

The intact protein mass was obtained using electrospray ionization mass spectrometry (ESI-MS) with a QTOF Ultima (Waters) mass spectrometer, operating under positive electrospray ionization mode, connected to an LC-20AD (Shimadzu) liquid chromatography unit. For tandem mass spectrometry, peptides were separated by nano-LC Ultimate 3000 high-performance liquid chromatography system (Thermo Fisher). The cross-linking mass spectra were analyzed with pLink 2.3. (Supp Ref 8-9).

### Example 2

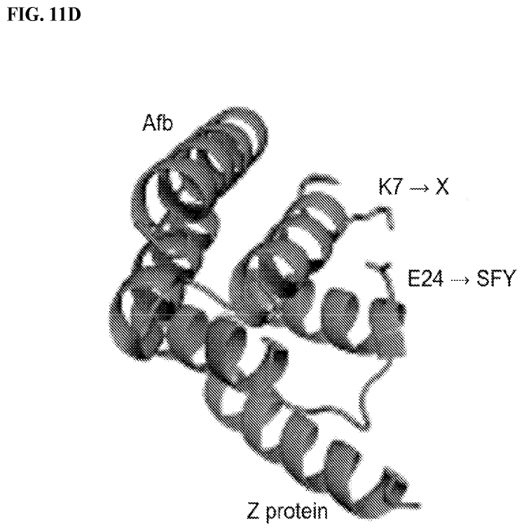

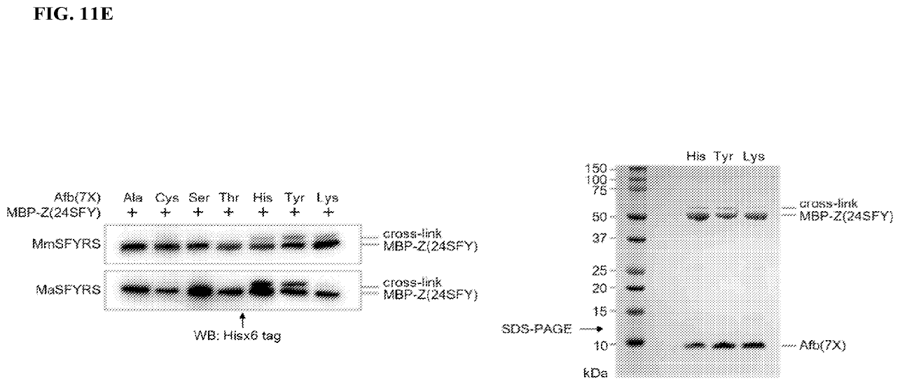

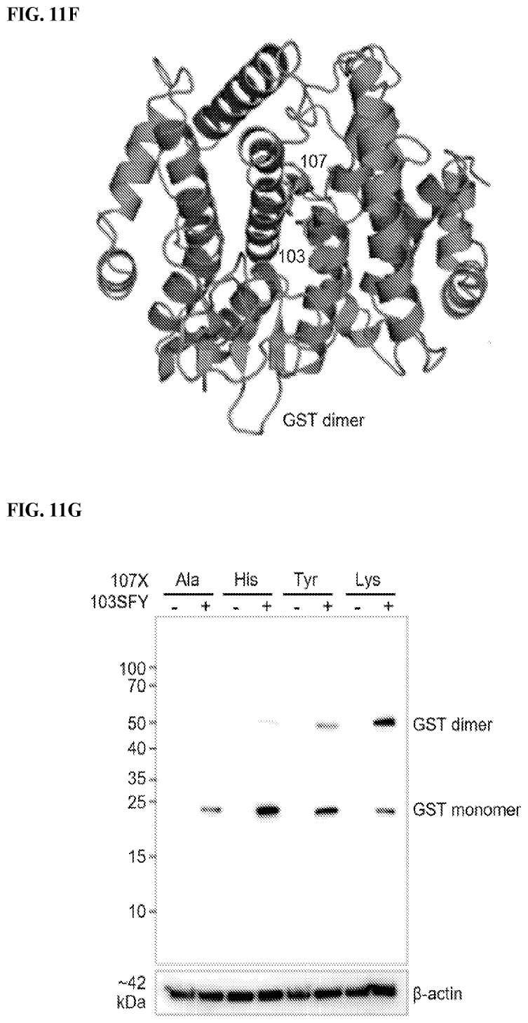

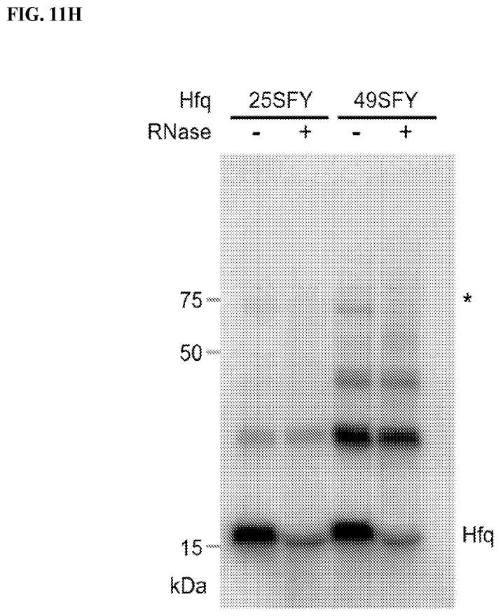

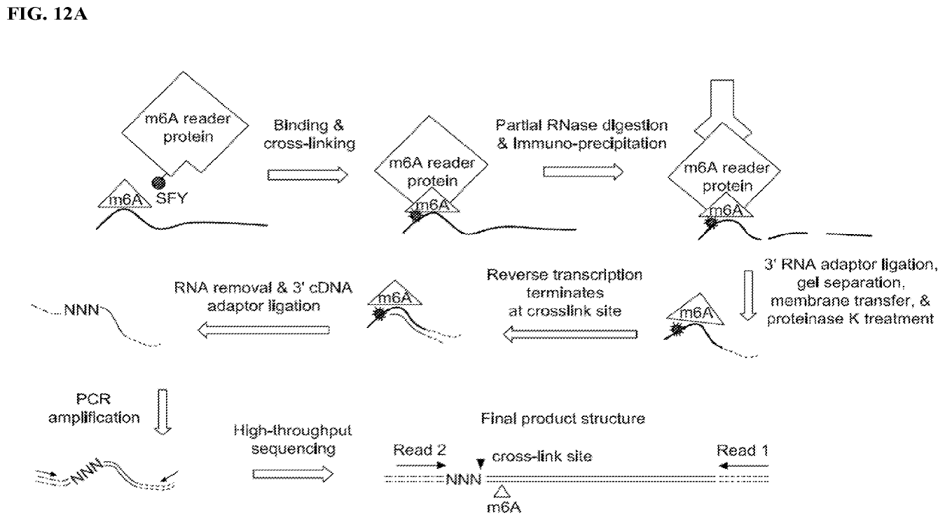

Here we demonstrate the incorporation of SFY (FIG. 11A) into proteins in mammalian cells and the ability of SFY to crosslink proximal nucleophilic amino acid sidechains via SuFEx directly in E. coli and mammalian cells.

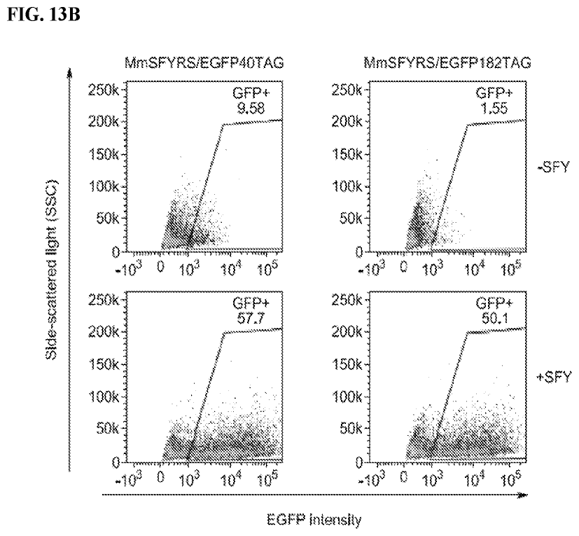

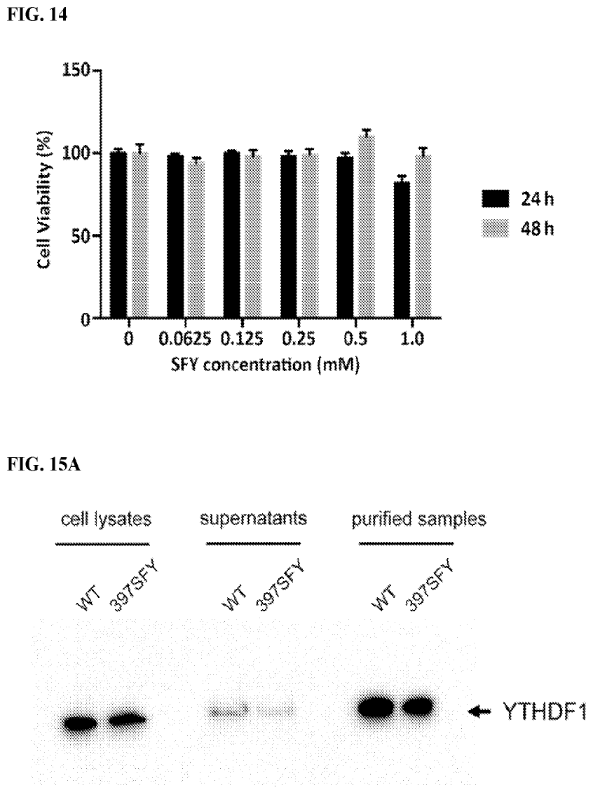

To test SFY incorporation in mammalian cells, we transfected HEK293 cells with plasmid pcDNA-EGFP-40TAG expressing EGFP gene containing a TAG codon at site Tyr40) and plasmid pNEU-MmSFYRS expressing the Mm-tRNA^{Pyl}/MmSFYRS. Fluorescence confocal microscopy showed that, in the presence of SFY, strong EGFP fluorescence was observed throughout the cells, and cell morphology remained normal (FIG. 11B), indicating SFY was incorporated at the TAG site to produce full-length EGFP. No fluorescence signal was detected when SFY was not added. HEK293 cells expressing pcDNA-EGFP-40TAG and Mm-tRNA^{Pyl}/MmSFYRS or Ma-tRNA^{Pyl}/MaSFYRS were further quantified by flow cytometry (FIGS. 11C, 13A-13B). Strong EGFP fluorescence was measured from cells only when SFY was added, and the fluorescence intensity increased with tRNA^{Pyl} copy number. In addition, we did not observe obvious toxicity of SFY to HEK293T cells (FIG. 14), a valuable property for in cell applications.

To determine which amino acid residues could react with SFY via proximity-enabled reactivity directly in cells, we coexpressed in E. coli the Z protein and an affibody (Afb) specifically binding it. Based on the crystal structure of Afb-Z complex, we introduced SFY at site 24 of the Z protein and various natural residues at site 7 of the affibody (FIG. 11D), placing the two residues in close proximity upon Afb-Z binding. (Wang et al. J. Am Chem. Soc. 140:4995-4999 (2018)). After expression of Afb(24SFY) and Z(7X) (X=target residue) for 6 h. cells were lysed and analyzed with Western blot under denatured conditions (FIG. 11E). Crosslinking bands corresponding to the adduct of Afb and Z were detected for target residue His. Tyr, and Lys, for both Mm-tRNA^{Pyl}/MmSFYRS and Ma-tRNA^{Pyl}/MaSFYRS. We then purified 6×His-tagged Z and Afb proteins from cells and analyzed with SDS-PAGE. Consistently, a protein band corresponding to the cross-linked Z with Afb was clearly observed for Afb-7Lys. Afb-7His, and Afb-7Tyr (FIG. 11E). We further tested if SFY could crosslink with these residues in mammalian cells. GST is a dimeric protein, whose structure shows that residue 103 of one monomer is close to residue 107 of the other monomer at the dimer interface (FIG. 11F), which has been used to determine proximity-enabled reactivity. (Liu et al. J. Am Chem. 141:9458-9462 (2019)). We incorporated SFY at site 103 of GST and mutated residue 107 to various target residues. HEK293T cells expressing these GST mutants were lysed and Western blotted to detect covalent GST dimer formation (FIG. 11G). Clearly SFY was shown to react with His. Tyr, and Lys placed in proximity in mammalian cells.

We also verified if SFY incorporated into Hfq could covalently capture RNA in E. coli cells. E. coli DH10B cells expressing Hfq(25SFY) or Hfq(49SFY) were lysed and analyzed with Urea-PAGE (FIG. 11H). Crosslinking bands were detected, which disappeared when samples were treated with RNase, indicating that Hfq(SFY) was able to crosslink RNAs in E. coli.

In addition, to check if SFY could cross-link all four nucleotides, we incubated 50 mM SFY with 50 mM different nucleoside monophosphates (NMPs: AMP, UMP, CMP, or GMP) at 37° C. for 16 hours. Cross-linking adducts of SFY with all four NMPs were detected using MS, confirming SFY could also cross-link nucleotides unbiasedly (data not shown).

### Example 3

An In Vivo Method for Detecting m⁶A in Mammalian Cells with Single-Nucleotide Resolution

N⁶-methyladenosine (m⁶A or m⁶A) is a widespread RNA modification that play important roles in the regulations and functions of mRNA. (Ref 39). Identification of the m⁶A sites in mRNA is critical for understanding m⁶A function. Although many m⁶A detection methods have been reported, the majority of them lack single nucleotide resolution and rely on the use of m⁶A-specific antibody, in which the recognition of m⁶A is in vitro in nature. (Refs 40-42). Specifically, we proposed to use a reader protein of m⁶A to recognize m⁶A sites on mRNA, and to incorporate a bioreactive SFY into the m⁶A binding site of the reader to cross-link nucleotides neighboring m⁶A (FIG. 12A). Expression of the reader-Uaa protein in cells would crosslink at m⁶A sites on RNA, enabling the recognition and capture of m⁶A motif in vivo. Immunoprecipitation of the reader protein followed with protease K digestion then release the captured RNAs for reverse transcription, adaptor ligation, and sequencing (FIG. 12A). The identified SFY-crosslinked nucleotides thus the reveal m⁶A site to be immediately adjacent.

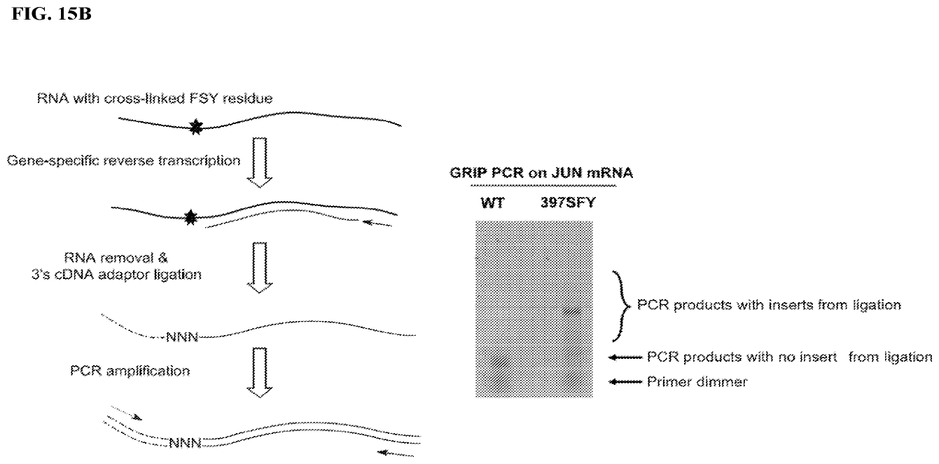

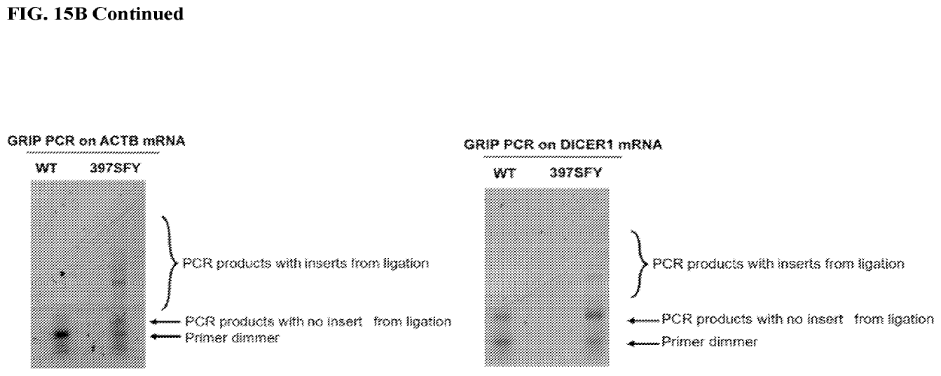

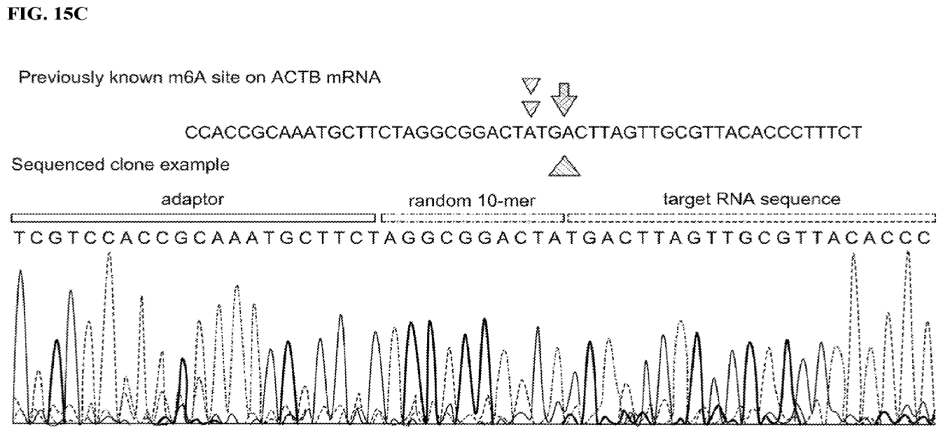

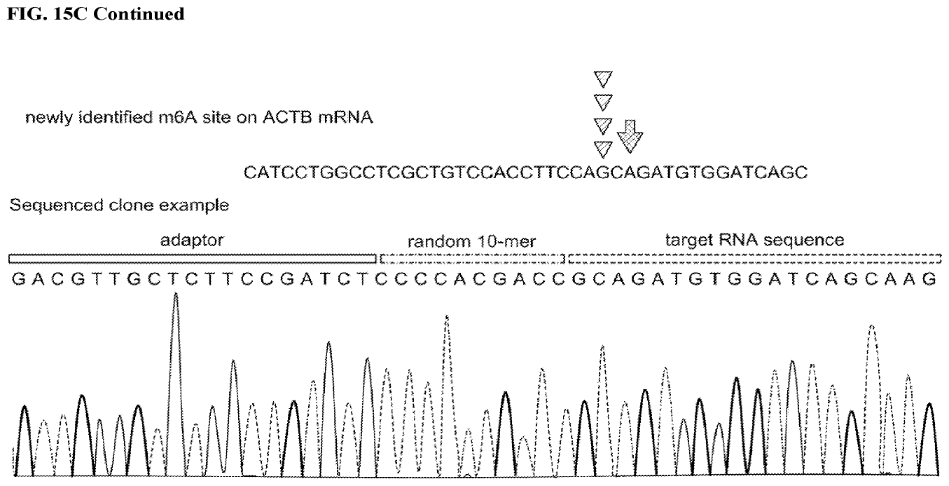

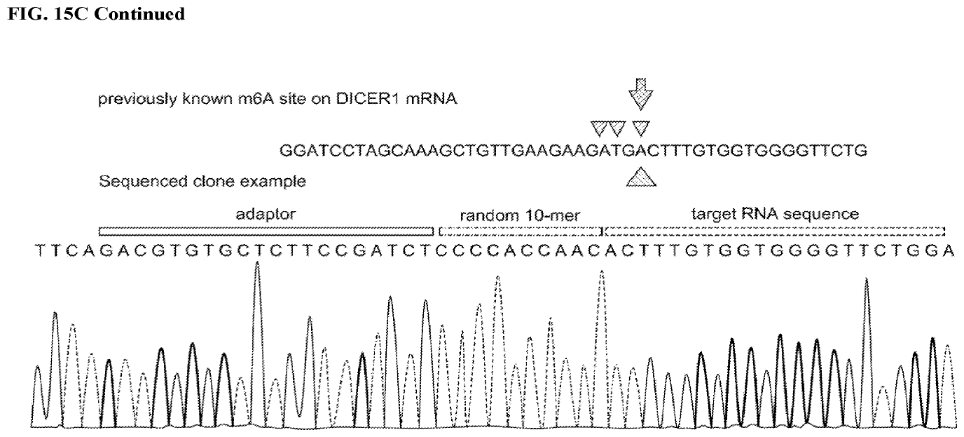

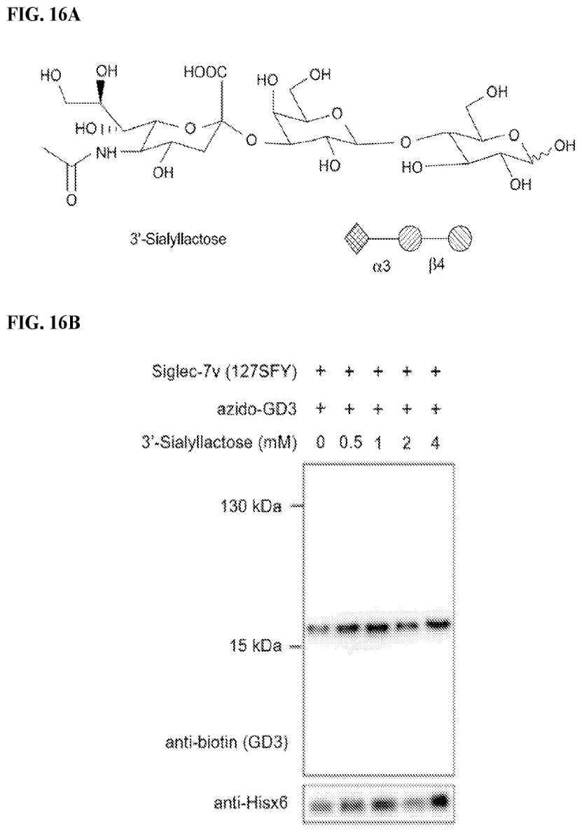

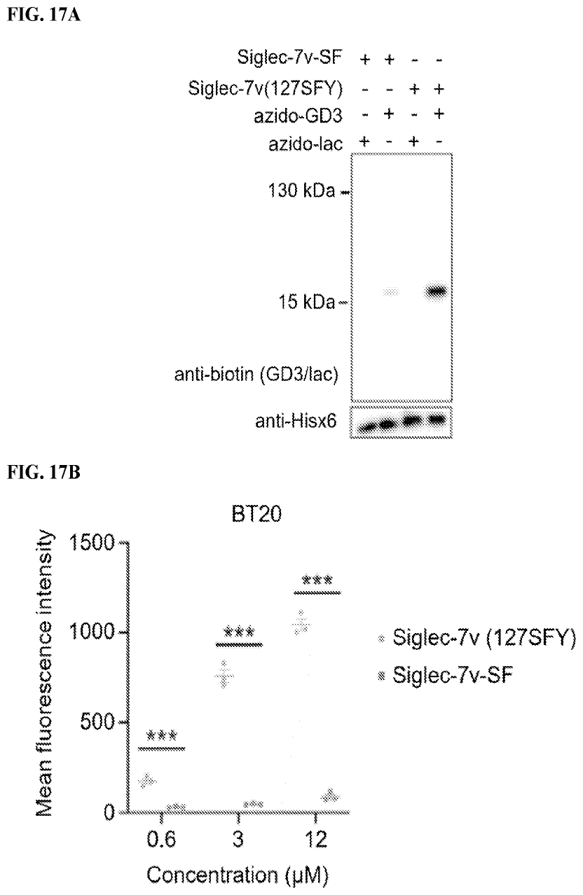

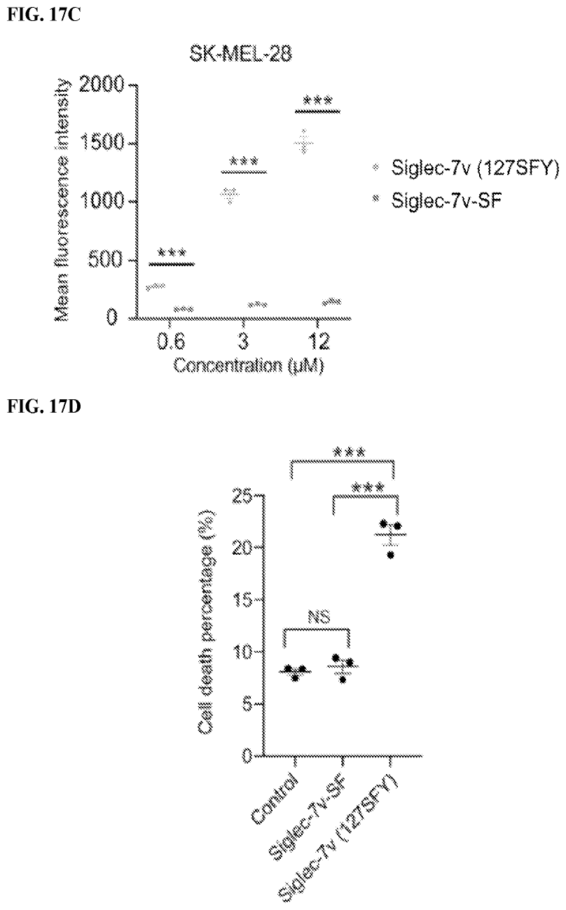

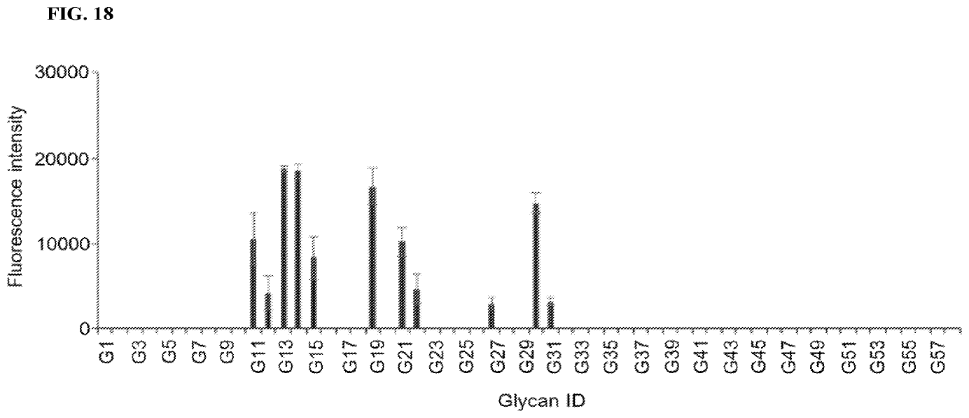

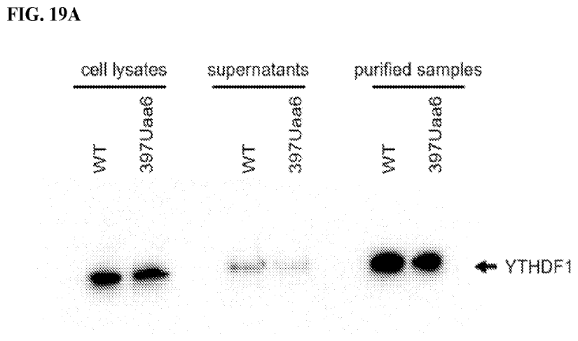

We used the YTH domain of human YTHDF1 protein, which is a conserved m⁶A reader. (Xu et al, J. Biol. Chem, 290:24902-24913 (2015); Meyer, Nat. Methods, 16:1275-1280) (2019)). Based on the crystal structure of YTHDF1 in complex with a 5-mer m⁶A RNA, we chose Tyr397, a residue next to the binding pocket of m⁶A, as the site for incorporating the bioreactive Uaa, to aim the Uaa side chain for targeting nucleotides upstream of m⁶A (FIG. 12B). (Xu et al, J. Biol. Chem, 290):24902-24913 (2015)). YTH-397SFY protein was expressed in HEK293T cells, followed by GRIP procedures (FIGS. 12A-12C). Three RNA regions from JUN, ACTBI, and BSG genes, containing known m⁶A sites, were reversely transcribed, ligated, and amplified with gene-specific primers, respectively. (Tang et al, Nucleic Acids Res, 49:D134-D143 (2020)). As expected, in final PCR products YTH-WT samples had no insertion, while YTH-397SFY samples showed distinct insertions for all three genes. After cloning and sanger sequencing of YTH-397SFY PCR products, we identified crosslinking sites at nucleotides 2-3 bp upstream of previously known m⁶A sites for all three genes (FIGS. 12D, 15C), confirming that this method was able to correctly identify m⁶A sites in mammalian cells as designed. Interestingly, apart from the known m⁶A sites, in the amplified regions of ACTB and JUN genes, we also identified two new m⁶A modification sites (FIGS. 12D, 15C), indicating that our method was able to identify m⁶A sites elusive to other methods. In all identified m⁶A sites, the assignment of m⁶A was unambiguous with single-nucleotide resolution.

### Example 4

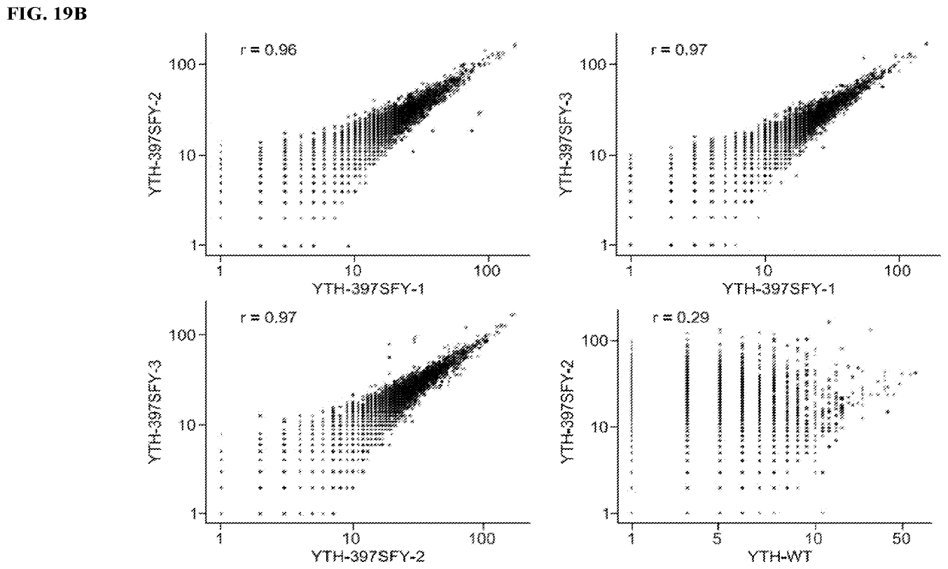

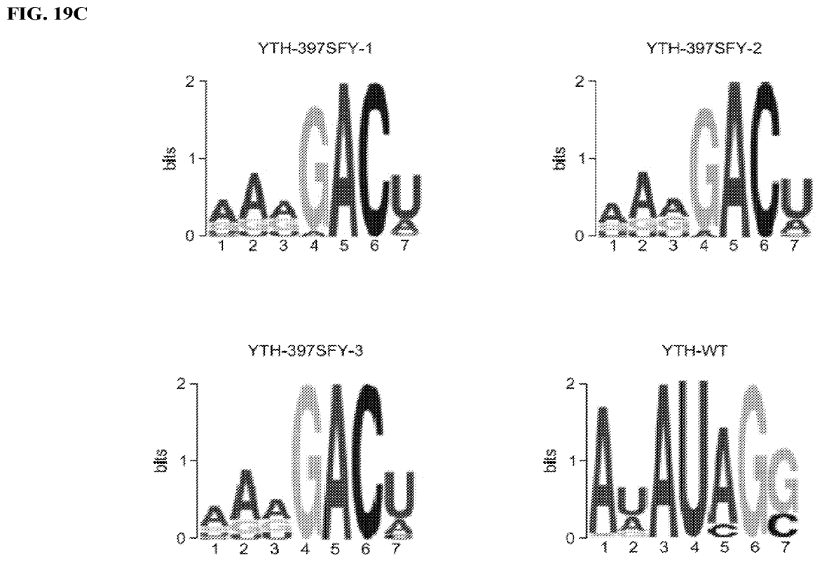

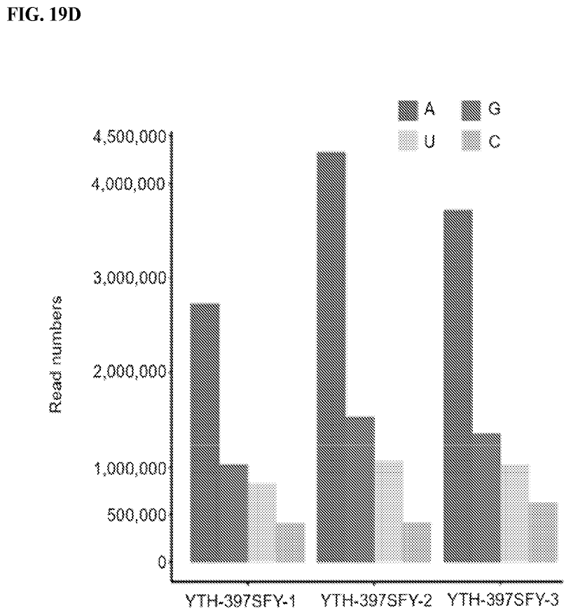

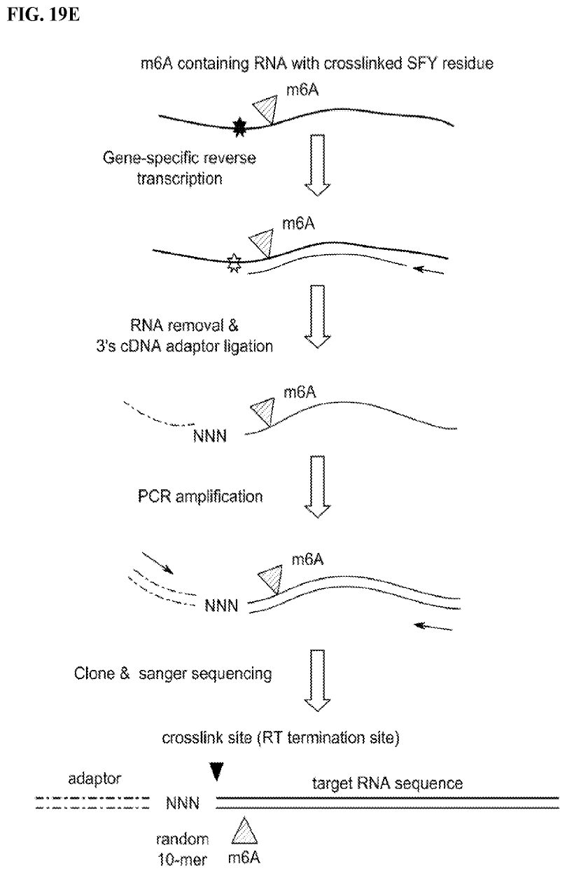

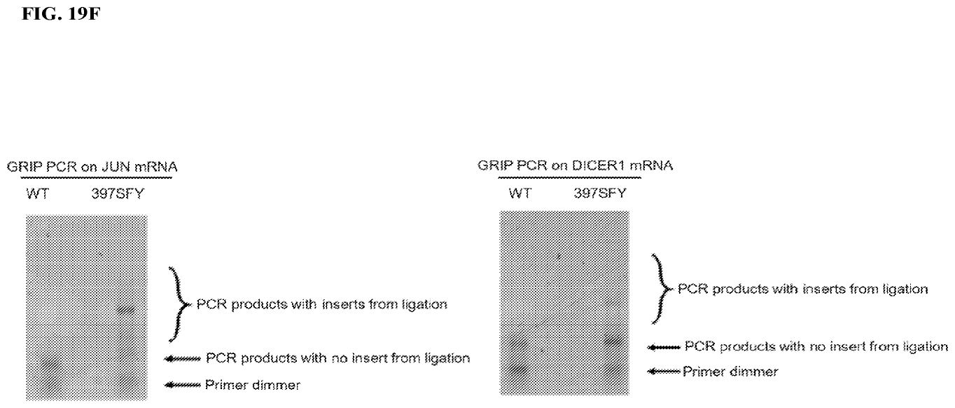

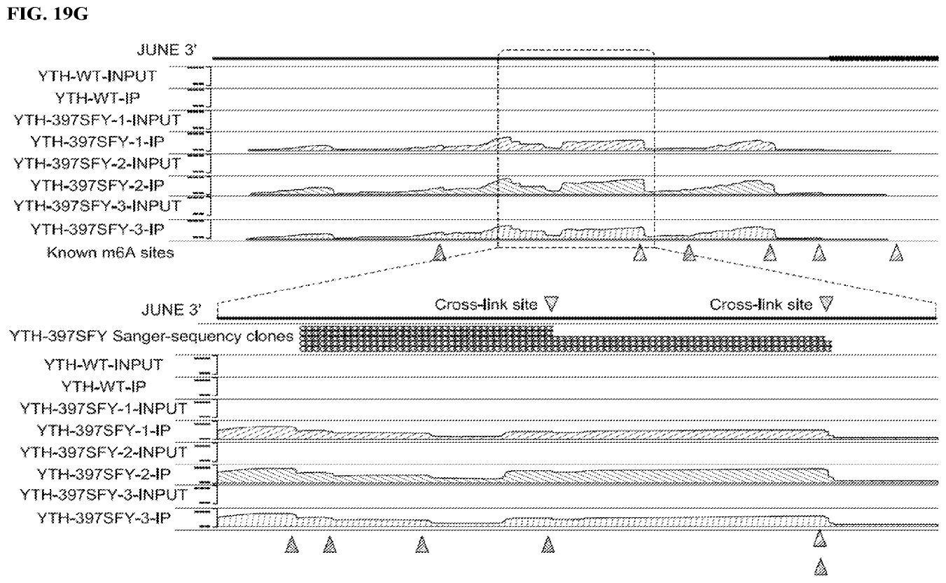

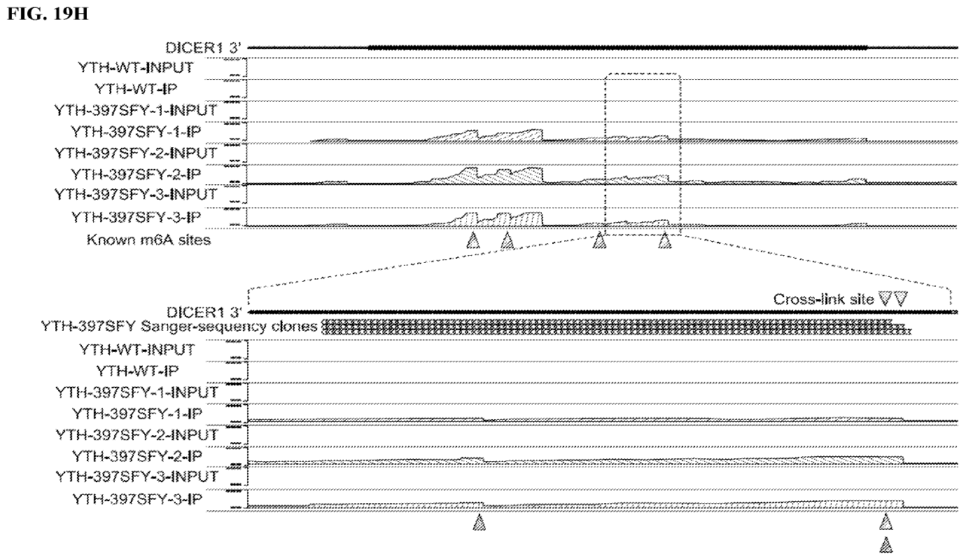

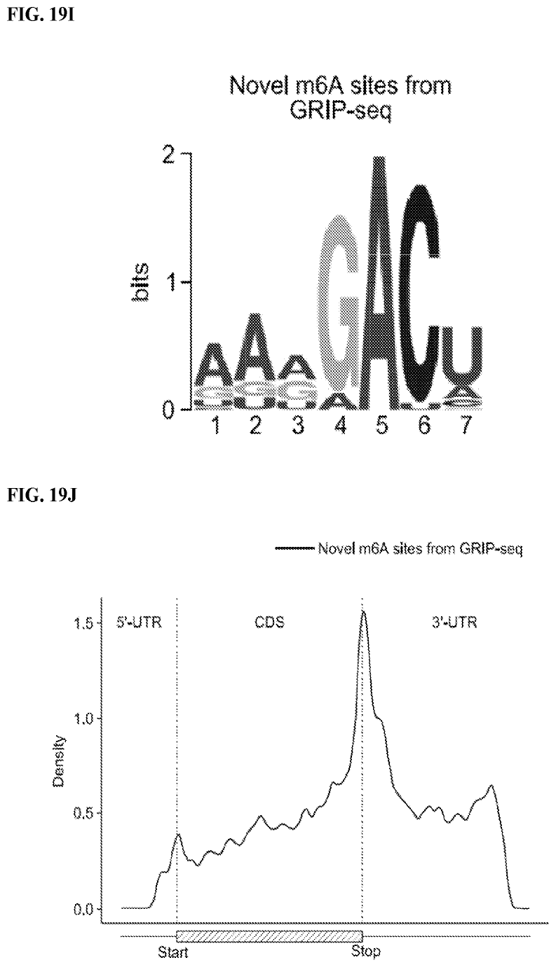

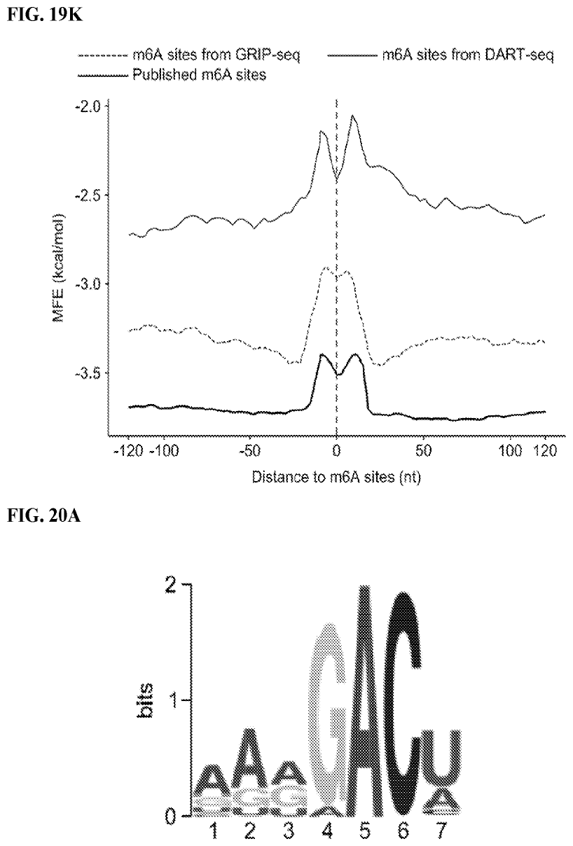

To detect endogenous m⁶A sites in mammalian cells throughout the transcriptome, we developed GRIP-seq through combining GRIP for m⁶A with high-throughput sequencing, enabling global identification of m⁶A sites in vivo with single-nucleotide resolution (FIG. 12A). In brief. HEK293T cells expressing YTH-397SFY protein (FIG. 19A) were lysed and treated with RNase to partially digest RNA into short fragments. After GRIP for these cell lysates (FIG. 19A), the purified protein-RNA cross-links were treated with proteinase K to release the cross-linked RNA fragments, which were converted into a cDNA library through adapting the enhanced CLIP protocol and then subjected to high-throughput sequencing. All m⁶A sites cross-linked by YTH-397SFY protein in vivo would be captured and identified by reverse transcription termination at the upstream cross-linked nucleotides. Van Nostrand et al. Nat. Methods 13:508-514 (2016)

We generated four pairs of GRIP-seq libraries. For each pair, we generated one library for the INPUT sample, which represents the RNA fragments from the whole cell lysate, and one library for the IP sample, which represents the RNA fragments cross-linked with the purified YTH proteins. These four pairs included one pair from HEK293 cells expressing YTH-WT protein serving as quality control, and three pairs from the three biological replicates of HEK293 cells expressing YTH-397SFY protein. For each library, around 10 to 35 million reads were obtained (data not shown). After removing adaptors, we first mapped the reads to the transcriptome. For IP libraries, we then used the CLIPPER algorithm to identify enriched peaks, which would represent RNA regions covering the reverse transcriptional termination sites and the cross-linking sites. Lovci et al. Nat. Struct. Mol. Biol. 20:1434-1442 (2013). While only 16,659 peaks were identified from the YTH-WT IP sample, 118, 746, 151, 153, and 139, 741 peaks were separately identified from the three YTH-397SFY IP samples. Aside from the drastic difference in total peak numbers between YTH-397SFY and YTH-WT IP samples, comparisons of each gene's peak numbers among the three YTH-397SFY IP samples indicated high reproducibility (Pearson's r>0.96. FIG. 19B), while the comparison of each gene's peak numbers between YTH-397SFY-2 and YTH-WT IP samples showed low correlation (Pearson's r=0.29, FIG. 19B). These results demonstrate that the peaks in YTH-397SFY replicates were specifically introduced through SFY incorporation and cross-linking.

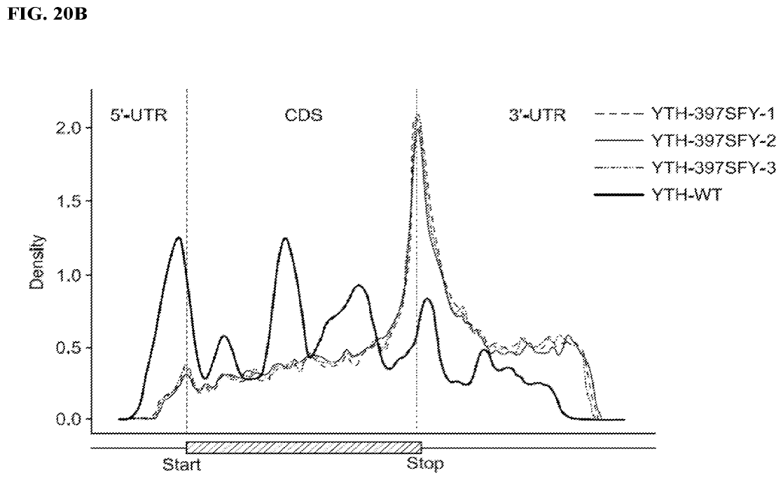

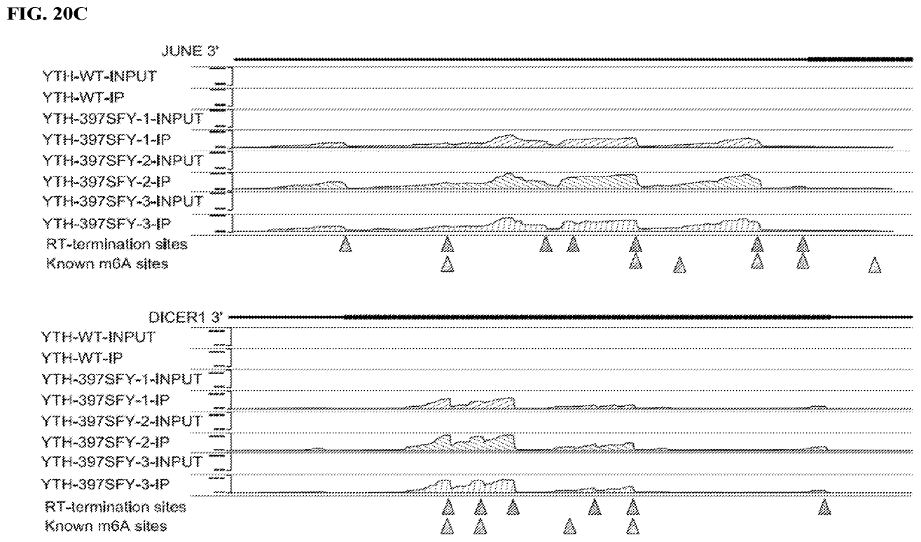

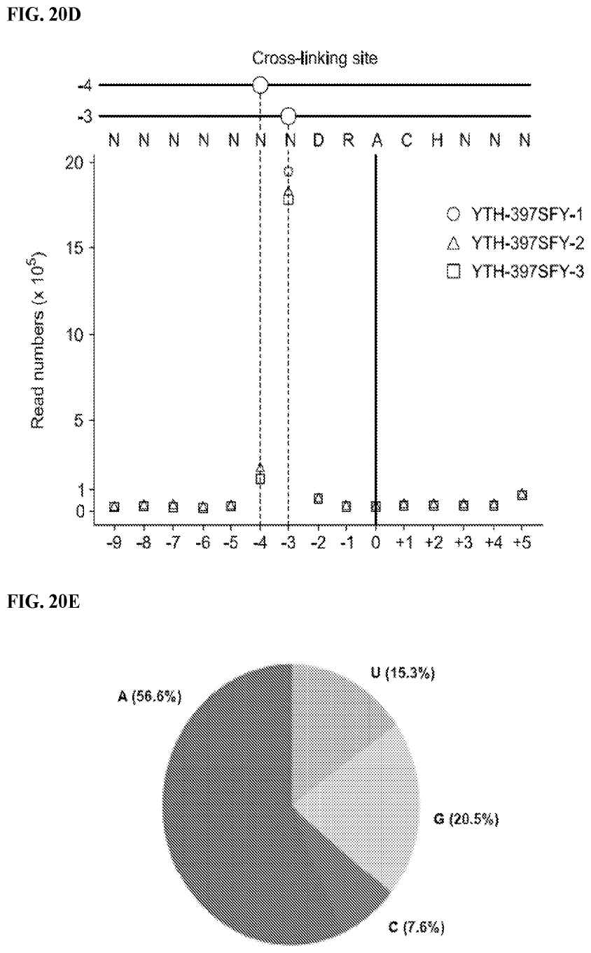

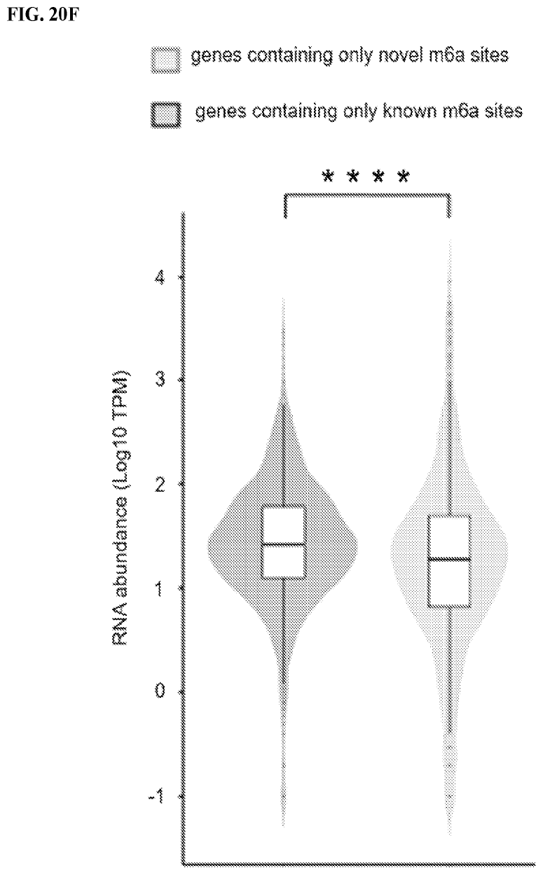

To determine if YTH-397SFY IP samples enriched m⁶A sites, we first identified the cross-linking-caused reverse-transcription-termination sites in these peaks (see materials and methods). Next we performed the sequence logo analysis of the sequences surrounding these reverse-transcription-termination sites. In all YTH-397SFY IP samples, the highest enriched motif was DRACH motif, which matched exactly the preferred consensus motif for m⁶A (FIGS. 20A, 19C). Meyer et al. Cell 149, 1635-1646 (2012); Dominissini et al. Nature 485, 201-206 (2012); Linder et al. Nat. Methods 12, 767-772 (2015); Meyer et al. Nat. Methods 16, 1275-1280) (2019). On the other hand, such DRACH motif could not be found enriched in the YTH-WT IP sample (FIG. 19C). In addition, the metagene profiles for the reverse-transcription-termination sites from the YTH-397SFY IP samples followed the typical distributions of m⁶A along mRNAs with strong enrichments around the stop codon (FIG. 20B), while those from the YTH-WT IP sample did not. Moreover, examinations of many individual RNAs, for example, JUN mRNA and DICER1 mRNA, showed that peaks from YTH-397SFY IP samples specifically enriched and terminated at previously identified m⁶A sites (FIG. 20C). Tang et al. Nucleic Acids Res. 49:D134-D143 (2020). These data indicated that in vivo expression of YTH-397SFY protein specifically cross-linked and enriched m⁶A modified RNA in mammalian cells.

In our design, the SFY residue in YTH-397SFY protein should cross-link with the nucleotide at the close upstream of m⁶A (FIGS. 20A-20B). To pinpoint which nucleotide next to m⁶A was cross-linked by YTH-397SFY, we analyzed the position of the cross-linked nucleotide relative to the DRACH motif in DRACH-containing reads from the enriched peaks. Indeed, if we denoted the middle A (m⁶A) in DRACH motif as position 0, cross-linking occurred at position −3 in 80.4% of DRACH-containing reads, and at position −4 in 9.3% of DRACH-containing reads (FIG. 20D), demonstrating that GRIP-seq could identify the m⁶A site with single-nucleotide-resolution. Moreover, analyzing the nucleotide composition of the cross-linked nucleotides revealed that SFY could cross-link with all four RNA nucleotides in vivo (FIGS. 20E, 19D), consistent with our in vitro experiment data.

Based on these features, we predicted a total of 13,968 m⁶A sites from the GRIP-seq data (data not shown). To further validate the m⁶A sites identified in GRIP-seq, we applied individual m⁶A GRIP procedures for two RNA regions that contain known m⁶A sites in JUN mRNA and DICER mRNA, employing gene-specific reverse transcription, ligation, amplification, and Sanger sequencing (FIG. 19E). As expected, in final PCR products YTH-WT samples had no insertion, while YTH-397SFY samples showed distinct insertions for both genes (FIG. 19F). After cloning and Sanger sequencing of YTH-397SFY PCR products, the identified cross-linking sites from Sanger sequencing matched the cross-linking sites from the GRIP-seq data (FIGS. 19G-19H), confirming that GRIP-seq was able to correctly identify m⁶A sites in mammalian cells as designed. Interestingly, apart from the known m⁶A site, in the amplified region of JUN gene, we also identified one novel m⁶A site using GRIP with Sanger sequencing capacity (FIG. 19G), which was also identified in the GRIP-seq data. Tang et al. Nucleic Acids Res. 49:D134-D143 (2020).

To further evaluate the capacity of GRIP-seq for identifying novel m⁶A sites, we compared the m⁶A sites from GRIP-seq with the known human m⁶A sites from the m⁶A-atlas, a comprehensive database for human m⁶A sites collected from seven published m⁶A-identification methods. Tang et al. Nucleic Acids Res. 49:D134-D143 (2020). The 6,686 m⁶A sites from GRIP-seq were known m⁶A sites that have been annotated in the m⁶A atlas, further validating GRIP-seq's ability in identifying m⁶A. Interestingly. 7,274 m⁶A sites from GRIP-seq have not been reported by any method in the m⁶A-atlas. Sequence logo analysis of these novel m⁶A sites from GRIP-seq showed strong enrichment of DRACH motif (FIG. 19I), and the metagene profile of these novel m⁶A sites also followed the typical distributions of m⁶A along mRNAs (FIG. 19J). These results demonstrated that GRIP-seq was able to uncover new m⁶A sites elusive to existing methods.

RNA secondary structure could alter the ability of RBPs' binding to target RNA and the reactivity of RNA nucleotides. To assess the potential effect of RNA secondary structure on GECX-RNA, we analyzed the predicted structural potential in RNA regions surrounding m⁶A sites from GRIP-seq and from the m⁶A-atlas, respectively. The m⁶A regions from GRIP-seq displayed a slightly less potential for stable secondary structures than the m⁶A regions from the m⁶A-atlas (FIG. 19K). However, most of m⁶A sites from the m⁶A-atlas were identified through detecting m⁶A on purified RNA molecules in vitro, while GRIP-Seq detected m⁶A on native cellular RNAs in vivo. In vitro purification and detection could disrupt stable in vivo RNA secondary structures and allow m⁶A more accessible for detection. We thus further compared the predicted structural potential in RNA regions surrounding m⁶A sites from GRIP-seq with those from DART-seq, another method that detects m⁶A sites in vivo. Interestingly, the m⁶A regions from GRIP-seq showed a much greater potential for secondary structures than those from DART-seq (FIG. 19K). Together, these results suggest that secondary structure folding in m⁶A regions from GRIP-seq is likely reflecting the in vivo binding preference of YTH domain for m⁶A RNAs. Meyer et al, Nat. Methods 16, 1275-1280 (2019); Tang et al. Nucleic Acids Res. 49:D134-D143 (2020). Sanchez de Groot et al. Nat. Commun. 10, 3246 (2019); Siegfried et al. Nat. Methods 11, 959-965 (2014); Gruber et al. Nucleic Acids Res. 36. W70-W74 (2008).

The proximity driven reactivity of GECX-RNA would enable cross-link with target RNA continuously whenever interaction occurs, allowing enriching the cross-linked product over a long period to improve the capture of interactions on low abundance RNAs. To determine if GRIP-seq was able to detect unknown m⁶A modifications on low abundance RNAs, we examined the abundance of mRNAs containing m⁶A sites detected by GRIP-seq. Among the m⁶A sites identified with GRIP-seq, 6,686 sites were also detected by previous methods and thus termed as “known m⁶A sites,” while 7,274 sites were detected by GRIP-seq only and termed as “novel m⁶A sites.” Between the group of genes containing only the known m⁶A sites and the group of genes containing only the novel m⁶A sites, we found that the genes containing only the novel m⁶A sites had significantly lower RNA abundances (FIG. 20F). Such low RNA abundances probably caused the neglect of these “novel” m⁶A sites in other m⁶A detection methods. Therefore, these results demonstrate that GRIP-seq was capable of capturing protein-RNA interactions on the low abundance RNAs.

### Materials and Methods for Examples 2-4

Cloning of pNEU-MmSFYRS-4xU6M15 plasmid. The MmSFYRS gene was amplified with primers HR-MmPylRS-NheI-F/HR-MmPylRS-NotI-R and ligated into pNEU-XYRS-4xU6M15 (derived from pNEU-hMbPylRS-4xU6M15, a gift from Irene Coin, Addgene plasmid #105830) which was linearized with NheI/NotI to generate pNEU-MmSFYRS-4xU6-M15.

Cloning of pNEU-MaSFYRS-NxU6-MaPylT (N=1 to 4) plasmids. The MaSFYRS and Ma-PylT expression cassettes were cloned into pNEU-XYRS-4xU6M15. Specifically, the U6 promoter was amplified from pNEU-XYRS-4xU6M15 with primers U6-F1/U6-R¹, and the evolved Ma-PylT(6) was amplified from pEvol-MaSFYRS with primers Ma-PylT(6)-F2/Ma-PylT(6)—R². The resulting fragments were joined together by overlapping PCR with primers U6-F1/Ma-PylT(6)-R² and then amplified again with primers HR-pNEU-tRNA-XhoI-F/HR-pNEU-tRNA-SalI-R to generate a monomeric U6-MaPylT expression cassette containing XbaI-XhoI and SalI restriction sites. The first monomeric U6-MaPylT expression cassette was ligated into pNEU-XYRS-4xU6M15 vector which was linearized with XhoI/Sa/I to generate pNEU-XYRS-1xU6-MaPylT. Then the MaSFYRS was amplified from pEvol-MaSFYRS with primers HR-Ma-SFYRS-NheI-F/HR-Ma-SFYRS-NotI-R and ligated into pNEU-XYRS-1xU6-MaPylT vector which was linearized with NheI/NorI to generate pNEU-MaSFYRS-1xU6-MaPylT. The second U6-MaPylT cassette was digested with XbaI/SalI and ligated into pNEU-MaSFYRS-1xU6-MaPylT vector that was linearized with XbaI/XhoI to generate pNEU-MaSFYRS-2xU6-MaPylT. Two more U6-MaPylT cassettes were tandemly introduced into the pNEU-MaSFYRS vector following the same procedure to construct the pNEU-MaSFYRS-4xU6-MaPylT.

Cross-linking of MBP-Z24SFY and Afb_{4A}-7X in live E. coli cells. The pET-Duet-Afb_{4A}-7X-MBP-Z24TAG (X=A, C, S, T, H, Y, or K)¹ was co-transformed with pEvol-MmSFYRS and pEvol-MaSFYRS² respectively into BL21(DE3) E. coli chemical competent cells. The transformants were plated on an LB-Amp100Cm34 agar plate and incubated overnight at 37° C. A single colony was inoculated into 5 mL of 2×YT-Amp100Cm34 and cultured overnight at 37° C. On the following day, 1 mL of overnight cell culture was diluted into 50 mL 2×YT-Amp100Cm34 and agitated vigorously at 37° C. When OD₆₀₀ reached 0.4˜0.6, the cell culture was induced with 0.5 mM IPTG and 0.2% arabinose in the presence of 1 mM SFY, and then incubated at 37° C. for 6 h. 1 mL of cell pellets were collected by centrifugation at 21000 g for 5 min at 4° C. and directly applied for immunoblot analysis. The rest of cell pellets were collected by centrifugation at 4200 g for 30 min at 4° C. The cross-linking products of MBP-Z24SFY and Afb_{4A}-7X (X═H, Y, or K) with affinity chromatography as described previously¹.

Cross-linking of GST-103SFY-107X in live mammalian cells. One day before transfection, 3×10⁵ HEK293T cells were seeded in a Greiner 6-well cell culture dish containing 2 mL of DMEM media with 10% FBS, and incubated at 37° C. in a CO₂ incubator. 1 μg of pcDNA-GST-103TAG-107X (X=A, H, Y or K)³ and 1 μg of pNEU-MmSFYRS-4xU6M15 were co-transfected into target cells using 5 μL of lipofectamine 2000 following the manufacturer's instructions. Six hours post transfection, the media were replaced with complete DMEM media with or without 1 mM SFY. The cells were incubated at 37° C. for additional 48 h, collected, and applied for immunoblot analysis.

Fluorescence confocal microscopy. One day before transfection, 3×10⁵ HEK293T cells were seeded in a Greiner 6-well cell culture dish containing 2 mL of DMEM media with 10% FBS, and incubated at 37° C. in a CO₂ incubator. Plasmids pcDNA-EGFP-40TAG (1 μg) and pNEU-MmSFYRS-4xU6M15 (1 μg) were co-transfected into target cells using 5 μL of lipofectamine 2000 following the manufacturer's instructions. Six hours post transfection, the media were replaced with complete DMEM media with or without 1 mM SFY. The cells were incubated at 37° C. for additional 24-48 h and imaged with Nikon Eclipse Ti confocal microscope.

FACS analysis of SFY incorporation. One day before transfection, 3×10⁵ HEK293T cells were seeded in a Greiner 6 well-cell culture dish containing 2 mL of DMEM media with 10% FBS, and incubated at 37° C. in a CO₂ incubator. Plasmids pcDNA-EGFP-40TAG (1 μg) and pNEU-MaSFYRS-NxU6-MaPylT (N=1 to 4) (1 μg) were co-transfected into target cells using 5 μL of lipofectamine 2000 following the manufacturer's instructions. Six hours post transfection, the media containing transfection complex were replaced with fresh DMEM media with 10% FBS in the presence or absence of 1 mM SFY. After incubation at 37° C. for 24-48 h, transfected cells were trypsinized and collected by centrifugation (1500 rpm, 5 min, r.t.). The cells were resuspended in 500 μL of FACS buffer (1×PBS, 2% FBS, 1 mM EDTA, 0.1% sodium azide, 0.28 μM DAPI) and analyzed by BD LSRFortessa™ cell analyzer.

Cell viability assay. 2×10⁴ cells/well of HEK293T cells were seeded in a 96-well plate. On the next day, the media were replaced with fresh DMEM media supplemented with 0, 0.0625, 0.125, 0.25, 0.5, or 1 mM of SFY. The SFY-treated and control cells were cultured for an additional 24-48 h at 37° C. and then analyzed with CellTiter-Blue R Cell Viability Assay following the manufacturer's instructions.

RNase treatment and detection for exogenous Hfq-expressing E. coli cells (Hfq-SFY samples). The procedure is the same as the RNase treatment and detection for exogenous Hfq-expressing E. coli cells(Hfq-WT and Hfq-FSY samples), with the following modifications: For the transformations, pBAD-Hfq TAG mutant plasmids (pBAD-Hfq-25TAG, pBAD-Hfq-49TAG) was co-transformed with pEvol-MmSFYRS into DH10B E. coli chemical competent cells, respectively. For the exogenous expression of Hfq-SFY proteins, the cell culture was induced with 0.2% arabinose and 1 mM SFY.

In vitro incubations of NMPs and SFY. 50 mM SFY (HCl salt) and 50 mM NMP was incubated in DI H2O. 50 mM NaOH was added to neutralize the HCl salt. The mixture was incubated at 37° C. for 48 h. Then the reaction mixture was diluted for 50 times in H₂O/acetonitrile (50/50, v/v, with 0.1% trifluoracetic acid) and subjected to mass spectrum analysis using positive mode. Mass spectrum analysis was performed on SCIEX MDS, 3200 Q TRAP system.

The molecular weight (MW) of addict products between SFY and NMP was calculated following this equation: MW (adduct product)=MW (SFY)+MW (NMP)−MW (HF).

| | | | | Expected | Observed |
|---|---|---|---|---|---|
| | | | Expected | MW of | MW of |
| | | | MW of | adduct | adduct |
| | MW of | MW of | adduct | products + | products + |
| | NMP | SFY | products | H⁺ | H⁺ |
| AMP | 347.0631 | 277.0420 | 604.0989 | 605.1062 | 605.0 |
| GMP | 363.0580 | | 620.0938 | 621.1011 | 620.8 |
| UMP | 324.0359 | | 581.0717 | 582.0789 | 581.7 |
| CMP | 323.0519 | | 580.0876 | 581.0949 | 580.9 |

Cloning of YTH domain from human YTHDF1 protein. To generate plasmids expressing YTH domain from human YTHDF1 protein with TwinStrep tag and HA tag at C-terminal in mammalian cells, three PCR products were prepared. Insert with YTHDF1 domain was amplified with primer pair of pc31-Hd3-YTHDF1-F and YTHDF1-2xstrep-R using cDNA reverse-transcribed from total RNA of HEK293T cells as template. Insert with TwinStrep tag was amplified with primer pair of 2xstrep-tag_Hs-F and 2xstrep-tag_Hs-R, pcDNA3.1 vector backbone was amplified with primer pair of pc31-HA-strep-F and pc31-Nde1-R using empty pcDNA3.1 vector as template. The final plasmid pcDNA3.1-HsYTHDF1-WT expressing wildtype YTHDF1 domain with TwinStrep tag and HA tag at C-terminal was cloned by ligating these three PCR products together using ClonExpress II one step cloning kit (Vazyme). To generate pcDNA3.1-HsYTHDF1-397TAG mutant plasmid, residue 397 of YTHDF1 gene in pcDNA3.1-HsYTHDF1-WT were mutated into an amber stop codon TAG using site-directed mutagenesis with following primers: YTHDF1-Y397TAG-F and YTHDF1-Y397TAG-R.

GRIP for in vivo m⁶A detection. HEK293T cells were plated in 15-cm plates and transfected with 15 μg of pcDNA3.1-HsYTHDF1 plasmids, with an additional 15 μg of pNEU-SFYRS plasmid (encoding SFY-tRNA synthetase-tRNA system for expression in mammalian cells) and 1 mM SFY for conditions involving YTHDF1-397SFY protein expression. Forty-eight hours after transfection, cells were washed twice with ice-cold PBS, and centrifuged to collect as cell pellets. Cells were lysed with 1.5 mL of 1×RIPA Buffer supplemented with protease inhibitors and RNase inhibitor. Cells were lysed on ice for 10 min and then passed through 26G-needles for 20 times to achieve full lysis. Lysates were then pelleted by centrifugation at 16,000 g for 10 min at 4° C. and the supernatants containing cleared lysates were used for pulldown with magnetic beads.

For Strep-Tactin® XT magnetic beads (Iba-lifesciences). 200 μL per sample of beads were pelleted by application of a magnet, and the supernatant was removed. Beads were washed twice with wash buffer (PBS buffer with 6 M Urea, 1 M NaCl, 1 mM DTT), and resuspended in 11.25 mL of wash buffer (PBS buffer with 6M Urea, 1 M NaCl, 1 mM DTT). 750 μL of sample lysate were added to beads and rotated overnight at 4° C.

After incubation with sample lysate, beads were pelleted, washed three times with 6M Urea. 1 M NaCl. PBS buffer, 1 mM DTT, wash once with PBS buffer with 1 M NaCl, wash once with PBS buffer, and then washed with DNase buffer (350) mM Tris-HCl (pH 6.5); 50 mM MgCl2; 5 mM DTT). Beads were resuspended in DNase buffer and TURBO DNase was added to a final concentration of 0.1 U/μL. DNase was shaking-incubated for 30 min at 37° C. Proteins were then digested by shaking-incubation with 50 μL of 5 mg/mL protease K, 2 M urea at 37° C. for 1 h. RNA was purified using QuickRNA micro prep kits.

RNA samples were reverse-transcribed with gene-specific RT primers targeting different cross-linking genes and regions (ACTB-m⁶A-1-RT. DICER1-m⁶A-1-RT, and JUN-m⁶A-1-RT, as listed in FIG. 9) with SuperScript IV First-Strand Synthesis System. The cDNA was treated with ExoSAP-IT to remove free primers, and then treated with NaOH to degrade RNA molecules. After clean-up with DynaBeads MyONE Silane, a 5′ linker (Rand3Tr3 adapter. FIG. 9) was ligated to cDNA molecules by T4 RNA ligase in on-beads solution with high concentration of PEG8000 at room temperature for 16 h. The ligated product was cleaned up again with DynaBeads MyONE Silane, and then amplified with primers targeting gene-specific regions and the 5′ linker (PCR primer pair for the GRIP region of ACTB RNA are primers pBADf-ACTB-m⁶A-1-pF and pBADr-eCLIP-Rand103tr3-pR; PCR primer pair for the GRIP region of DICER1 RNA are primers pBADf-DICER1-m⁶A-1-pF and pBADr-eCLIP-Rand103tr3-pR; and PCR primer pair for the GRIP region of JUN RNA are primers pBADf-JUN-m⁶A-1-pF and pBADr-eCLIP-Rand103tr3-pR). The PCR product was separated on agarose gel. The insertion bands were cut out, purified and cloned into pBAD vector, transformed into DH10B competent cells, and plated onto LB-Amp100 agar plate and incubated overnight at 37° C. Plasmids were then extracted from colonies and sequenced. The sequenced inserts from plasmids were aligned to target RNA regions (ACTB. DICER1, or JUN), the ligation sites of 5′ linker represent the cross-linking sites of YTHDF1-397SFY proteins on target RNA molecules, thus also representing m⁶A sites on target RNA molecules.

Library preparation for GRIP-seq. HEK293T cells were plated in 15-cm plates and transfected with 15 pg of pcDNA3.1-HsYTHDF1 plasmids, with an additional 15 pg of pNEU-SFYRS plasmid (encoding SFY-synthetase-tRNA system for expression in mammalian cells) and 1 mM SFY for conditions involving YTHDF1-397SFY protein expression. Forty-eight hours after transfection, cells were washed twice with ice-cold PBS, and centrifuged to collect as cell pellets. The library preparation procedure for GRIP-seq was similar to the protocol from eCLIP. In brief, the cell pellets were lysed in 1 mL of eCLIP lysis buffer, partially digested with RNase I (Invitrogen). 20 pL of the cell lysate was stored as “INPUT” sample for subsequent direct library preparation (similar as in eCLIP protocol). Van Nostrand. Nat. Methods. 14:508-514 (2016). The remainder of the cell lysate (about 1 mL) was immunoprecipitated using 200 pL of pre-washed strep-tactin-XT magnetic beads (Iba-lifesciences) targeting 2xStrep-tag sequence fused at C-terminal of YTH proteins, and stringently washed (twice with high-salt denaturing buffer (PBS buffer with 6 M Urea. 1 M NaCl. 1 mM DTT) and twice with PBS buffer). After dephosphorylation with FastAP (ThermoFisher) and T4 PNK (NEB), a barcoded RNA adaptor (1:1 mixed RNA_X1Aand RNA_X1B adaptors. Table S1) was ligated to the 3′ end (T4 RNA Ligase, NEB) of cross-linked and co-purified RNA. Ligations were performed on-bead. Next, Samples were run on protein gels and transferred to nitrocellulose membranes. On the membranes, the regions containing YTH protein-RNA cross-links were excised (membrane regions 75 kDa above the YTH protein) and treated with proteinase K to release the cross-linked RNA. RNA was then reverse-transcribed with SuperScript IV reverse transcriptase (ThermoFisher) and AR17 primer (Table S1), and treated with ExoSAP-IT (ThermoFisher) to remove excess oligonucleotides. A second DNA adaptor (Rand3Tr3 adaptor, Table S1) was then ligated to the 3′ end of the cDNA fragment (T4 RNA Ligase, NEB). After cleanup (Dynabeads MyOne Silane, ThermoFisher), an aliquot of each sample was first subjected to qPCR for determining the proper number of PCR cycles. Then, the remainder was amplified (Phanta Max Super-Fidelity DNA Polymerase, Vazyme) with a pair of PCR primer for final library amplification (P1A-0N-F and P1A-0N-R, “N” represents the specific index for different sample, Table S1) and size selected via agarose gel electrophoresis. Samples were sequenced on the Illumina NovaSeq S4 platform with paired-end 2×100 format.

TABLE S1

| | | SEQ ID |
|---|---|---|
| Name | Sequence | NO: |
| RNA_X1A | GrUrCrGrUrGrUrArG/3SpC3//5PHOS/rArArUrArGrCrArNr | 9 |
| | NrNrNrNrArGrArUrCrGrGrArArGrArGrCrG | |
| RNA_X1B | rUrCrGrUrGrUrArG/3SpC3/ | 10 |
| AR17 | ACACGACGCTCTTCCGAAATGATACGGCGACCACCG | 11 |
| | AGATCTACACNNNNNNNNACACTCT | |
| P1A0NF | TTCCCTACACGACGCTCTTCCGATCTCAAGCAGAAGA | 12 |
| | CGGCATACGAGATNNNNNNNNGTGACTGGAGTT | |
| PIA0NR | CAGACGTGTGCTCTTCCGATCT | 13 |

Table S1 Note: for rand103Tr3 adaptor, RNA_X1A and RNA_X1B, “N” and “rN” represented random nucleotides. Note: for P1A0NF and P1A0NR, “NNNNNNNN” represented the library index sequences for illumina sequencing. Note: “r” preceding a letter refers to ribose or RNA; and “3SpC3” or “/SpC3/” refers to a linking group.

### Data Analysis for GRIP-Seq

Read Processing. After standard illumina Hiseq demultiplexing, GRIP-seq libraries were first processed with Fastp tool to remove PCR duplications and cut illumina adaptors, and then processed with Cutadapt tool to remove the GRIP-seq adaptors and retrieve the inserted RNA sequences according to the following GRIP-seq final library structure. Library structure with X1A_adaptor: (Read1) NNNNNCCTATAT-INSERT-NNNNNNNNNN (Read2) Library structure with X1B_adaptor: (Read1) NNNNNTGCTATT-INSERT-NNNNNNNNNN (Read2) Note: “N” in library structures representing random nucleotides. Chen et al, Bioinformatics, 34:1884-1890 (2018); Martin, EMBnet.journal 17:10-12 (2011).

Read mapping. Reads were mapped with STAR to the human genome (hg19) by default setting. Dobin et al, Bioinformatics, 29:15-21 (2013).

Identification of m⁶A clusters and reverse-transcription-termination sites. After mapping, CLIPper was applied on the mapped reads 2 (reads 2 is the read starting right after the cross-linking site (FIG. 12A)) with options “—FDR 0.01—poisson-cutoff 1e-10—minreads 5—binomial 0.01” to identify the read clusters. After cluster identification, the precise reverse-transcription-termination sites of clusters were identified by scanning all sites within the cluster using the following criteria: If the read number covering the scanned site was greater than 1.5-fold bigger and had greater than 40 reads than the read number covering the neighboring site, this site will be designated as reverse-transcription-termination site.

Metagene and motif analyses. After reverse-transcription-termination site was identified, the sequences spanning a region 10-nt up- and downstream of termination sites were extracted and used as input for motif discovery using MEME. Metagene analysis was performed with reads mapped within m⁶A clusters using metaPlotR. Bailey et al. Nucleic Acids Res. 37:W202-W208 (2009); Olarerin-George et al. Bioinformatics, 33:1563-1564 (2017).

Analysis of cross-linking site positions relative to the m⁶A motif. Reads 2 overlapping with regions containing motif DRACH from motif analysis were extracted. The numbers of reads 2 starting right after each position relative to DRACH motif (the middle A in the motif was designated as position 0 were calculated and plotted.

Analysis of nucleotide composition at cross-linking sites. For reads in m⁶A clusters, the cross-linking sites were designated as the nucleotides 1-nt upstream of read 2 starting positions.

Identification of m⁶A sites. After the position of cross-linking site relative to m⁶A motif was revealed, the precise m⁶A sites were assigned according to the distance to the revers-transcription-termination sites.

Secondary structure analysis around m⁶A sites. The coordinates of published m⁶A sites were from m⁶A-atlas database. The coordinates of m⁶A sites from DART-seq were m⁶A sites from “HEK293T, DART-seq, control sample” in m⁶A-atlas database. For each m⁶A site, a sliding window of 30 nucleotides with a step of 3 nucleotides was used to calculate RNA minimum fold free energy (MFE) spanning the regions 120-nt up- and downstream of m⁶A sites. For each window, MFE was calculated by ViennaRNA, using default parameters. For m⁶A sites from different datasets, a mean MFE in each window was calculated by averaging MFE values of the windows in the same position. Tang et al, Nucleic Acids Res. 49:D134-D143 (2020); Lorenz et al, Algorithms Mol. Biol. 6:26 (2011).

Code availability. Custom code used is available at https://github.com/Shall-We-Dance/GRIP-seq.

Accession codes. All GRIP-seq data are available in SRA database with Accession number: PRJNA797913.

### Informal Sequence Listing

In SEQ ID NO:1-4, the first occurrence of the bold underlined K refers to lysine at position 104 (or a position corresponding to position 104); the second occurrence of the bold underlined K refers to lysine at position 127 (or a position corresponding to position 127); and the bold underlined N refers to asparagine at position 129 (or a position corresponding to position 129).

```
(Sialic Acid Binding Ig Like Lectin 7; UniProtKB No. Q9Y286)
SEQ ID NO: 1
MLLLLLLPLL WGRERVEGQK SNRKDYSLTM QSSVTVQEGM CVHVRCSFSY
                60 70 80 90 100
PVDSQTDSDP VHGYWFRAGN DISWKAPVAT NNPAWAVQEE TRDRFHLLGD
              110 120 130 140 150
PQTKNCTLSI RDARMSDAGR YFFRMEKGNI KWNYKYDQLS VNVTALTHRP
              160 170 180 190 200
NILIPGTLES GCFQNLTCSV PWACEQGTPP MISWMGTSVS PLHPSTTRSS
              210 220 230 240 250
VLTLIPQPQH HGTSLTCQVT LPGAGVTTNR TIQLNVSYPP QNLTVTVFQG
              260 270 280 290 300
EGTASTALGN SSSLSVLEGQ SLRLVCAVDS NPPARLSWTW RSLTLYPSQP
              310 320 330 340 350
SNPLVLELQV HLGDEGEFTC RAQNSLGSQH VSLNLSLQQE YTGKMRPVSG
              360 370 380 390 400
VLLGAVGGAG ATALVFLSFC VIFIVVRSCR KKSARPAADV GDIGMKDANT
              410 420 430 440 450
IRGSASQGNL TESWADDNPR HHGLAAHSSG EEREIQYAPL SFHKGEPQDL
              460
SGQEATNNEY SEIKIPK
(sialoglycan binding V-set domain of Siglec-7)
SEQ ID NO: 2
                                                                                        60 70
QK SNRKDYSLTM QSSVTVQEGM CVHVRCSFSY PVDSQTDSDP VHGYWFRAGN
                  80 90 100 110 120
DISWKAPVAT NNPAWAVQEE TRDRFHLLGD PQTKNCTLSI RDARMSDAGR
              120 130 140
YFFRMEKGNI KWNYKYDQLS VNVTALTH
(sialoglycan binding V-set domain of Siglec-7)
SEQ ID NO: 3
                                                                                          60 70
MQK SNRKDYSLTM QSSVTVQEGM SVHVRCSFSY PVDSQTDSDP VHGYWFRAGN
                80 90 100 110 120
DISWKAPVAT NNPAWAVQEE TRDRFHLLGD PQTKNCTLSI RDARMSDAGR
              130 140
YFFRMEKGNI KWNYKYDQLS VNVTALTHHH HHHH
(sialoglycan binding V-set domain of Siglec-7)
SEQ ID NO: 4
                                                                                        60 70
QK SNRKDYSLTM QSSVTVQEGM SVHVRCSFSY PVDSQTDSDP VHGYWFRAGN
                80 90 100 110 120
DISWKAPVAT NNPAWAVQEE TRDRFHLLGD PQTKNCTLSI RDARMSDAGR
              130 140
YFFRMEKGNI KWNYKYDQLS VNVTALTH
(wild-type amino acid sequence of Methanosarcina mazei PylRS)
SEQ ID NO: 5
MDKKPLNTLI SATGLWMSRT GTIHKIKHHE VSRSKIYIEM ACGDHLVVNN
SRSSRTARAL RHHKYRKTCK RCRVSDEDLN KFLTKANEDQ TSVKVKVVSA
PTRTKKAMPK SVARAPKPLE NTEAAQAQPS GSKFSPAIPV STQESVSVPA
SVSTSISSIS TGATASALVK GNTNPITSMS APVQASAPAL TKSQTDRLEV
LLNPKDEISL NSGKPFRELE SELLSRRKKD LQQIYAEERE NYLGKLEREI
TRFFVDRGFL EIKSPILIPL EYIERMGIDN DTELSKQIFR VDKNFCLRPM
LAPNLYNYLR KLDRALPDPI KIFEIGPCYR KESDGKEHLE EFTMLNFCQM
GSGCTRENLE SIITDFLNHL GIDFKIVGDS CMVYGDTLDV MHGDLELSSA
VVGPIPLDRE WGIDKPWIGA GFGLERLLKV KHDFKNIKRA ARSESYYNGI STNL
(mutant sequence of Methanosarcina mazei PylRS)
SEQ ID NO: 6
MDKKPLNTLI SATGLWMSRT GTIHKIKHHE VSRSKIYIEM ACGDHLVVNN
SRSSRTARAL RHHKYRKTCK RCRVSDEDLN KFLTKANEDQ TSVKVKVVSA
PTRTKKAMPK SVARAPKPLE NTEAAQAQPS GSKFSPAIPV STQESVSVPA
SVSTSISSIS TGATASALVK GNTNPITSMS APVQASAPAL TKSQTDRLEV
LLNPKDEISL NSGKPFRELE SELLSRRKKD LQQIYAEERE NYLGKLEREI
TRFFVDRGFL EIKSPILIPL EYIERMGIDN DTELSKQIFR VDKNFCLRPM
LAPNLLNYAR KLDRALPDPI KIFEIGPCYR KESDGKEHLE EFTMLAFMQM
GSGCTRENLE SIITDFLNHL GIDFKIVGDS CMVYGDTLDV MHGDLELSSA
VVGPIPLDRE WGIDKPTIGA GFGLERLLKV KHDFKNIKRA ARSESYYNGI STNL
(wild-type amino acid sequence of Methanomethylophilus alvus PylRS)
SEQ ID NO: 7
MTVKYTDAQI QRLREYGNGT YEQKVFEDLA SRDAAFSKEM SVASTDNEKK
IKGMIANPSR HGLTQLMNDI ADALVAEGFI EVRTPIFISK DALARMTITE
DKPLFKQVFW IDEKRALRPM LAPNLYSVMR DLRDHTDGPV KIFEMGSCER
KESHSGMHLE EFTMLNLVDM GPRGDATEVL KNYISVVMKA AGLPDYDLVQ
EESDVYKETI DVEINGQEVC SAAVGPHYLD AAHDVHEPWS GAGFGLERLL
TIREKYSTVK KGGASISYLN GAKIN
(mutant amino acid sequence of Methanomethylophilus alvus PylRS)
SEQ ID NO: 7
MTVKYTDAQI QRLREYGNGT YEQKVFEDLA SRDAAFSKEM SVASTDNEKK
IKGMIANPSR HGLTQLMNDI ADALVAEGFI EVRTPIFISK DALARMTITE
DKPLFKQVFW IDEKRALRPM LAPNLLSVAR DLRDHTDGPV KIFEMGSCER
KESHSGMHLE EFTMLALMDM GPRGDATEVL KNYISVVMKA AGLPDYDLVQ
EESDVYKETI DVEINGQEVC SAAVGPHYLD AAHDVHEPTS GAGFGLERLL
TIREKYSTVK KGGASISYLN GAKIN
(DNA sequence for WT siglec 7v protein)
SEQ ID NO: 8
ATGCAGAAAAGTAATCGTAAAGATTATAGCCTGACCATGCAGAGCAGCGTGACCGT
TCAGGAAGGTATGTCTGTTCATGTGCGTTGCAGTTTTAGCTATCCGGTTGATAGCCA
GACCGATAGCGATCCGGTGCATGGCTATTGGTTTCGCGCCGGTAATGATATTAGTTG
GAAAGCCCCGGTTGCCACCAATAATCCGGCATGGGCAGTGCAGGAAGAAACCCGC
GATCGCTTTCATCTGCTGGGTGACCCGCAGACCAAAAATTGTACCCTGAGTATTCGC
GATGCACGTATGAGTGATGCAGGCCGCTATTTCTTTCGCATGGAAAAAGGTAATAT
CAAATGGAATTACAAGTACGATCAGCTGAGTGTTAATGTTACCGCCTTGACCCACC
ATCACCATCACCATCAC
```

### REFERENCES

1. Sears et al, Angew. Chem. Int. Ed. Engl. 38, 2300-2324 (1999).; 2. Spiro, Glycobiology 12, 43R-56R (2002).; 3. Fuster et al, Nat. Rev. Cancer 5, 526-542 (2005). 4 Stowell et al, Annu. Rev. Pathol. 10, 473-510 (2015).; 5. Imberty, A. & Prestegard, J. H. Structural Biology of Glycan Recognition. (2015). In “Essentials of Glycobiology”, 3Rd Ed. doi:10.1101/glycobiology.3e.030; 6. Nelson et al, Annu. Rev. Cell Dev. Biol. 11, 601-631 (1995).; 7. Sterner et al, ACS Chem. Biol. 11, 1773-1783 (2016).; 8. Polonskaya et al, Curr. Opin. Immunol. 59, 65-71 (2019).; 9. Xiang, Z. et al. Nat. Methods 10, 885-888 (2013).; 10. Wang, L. N. Biotechnol. 38, 16-25 (2017).; 11. Yang, B. et al. Nat. Commun. 8, 2240 (2017).; 12. Li, Q. et al. Cell 182, 85-97.e16 (2020).; 13. Jäger, M. & Minnaard, A. J. Chem. Commun. 52, 656-664 (2016).; 14. Wang et al, Chem. Sci. 4, 3381-3394 (2013).; 15. Falco, M. et al. J. Exp. Med. 190, 793-802 (1999).; 16. Crocker et al, Nat. Rev. Immunol. 7, 255-266 (2007).; 17. Yamaji et al, J. Biol. Chem. 277, 6324-6332 (2002).; 18. Attrill, H. et al. Biochem. J. 397, 271-278 (2006).; 19. Macauley et al, Nat. Rev. Immunol. 14, 653-666 (2014).; 20. Yang, B. et al. Proc. Natl. Acad. Sci. U.S.A. 115, 11162-11167 (2018).; 21. Hoppmann et al, Chem. Commun. 52, 5140-5143 (2016).; 22. Xiang, Z. et al. Angew. Chem. Int. Ed. Engl. 53, 2190-2193 (2014).; 23. Chen, X. H. et al. ACS Chem. Biol. 9, 1956-1961 (2014).; 24. Hoppmann, C., Maslennikov, I., Choe, S. & Wang, L. In Situ Formation of an Azo Bridge on Proteins Controllable by Visible Light. J. Am. Chem. Soc. 137, 11218-11221 (2015).; 25. Wang, N, et al. J. Am. Chem. Soc. 140, 4995-4999 (2018).; 26. Liu, J. et al. J. Am. Chem. Soc. 141, 9458-9462 (2019).; 27. Liu, J. et al. J. Am. Chem. Soc. 142, 17057-17068 (2020).; 28. Liu, J. et al. Angew. Chem. Int. Ed. Engl. 58, 18839-18843 (2019).; 29. Yu, H. et al. J. Am. Chem. Soc. 131, 18467-18477 (2009).; 30. Yu et al, J. Oleo. Sci. 60, 537-544 (2011).; 31. Krengel et al, Front. Immunol. 5, 325 (2014).; 32. Attrill, H. et al. J. Biol. Chem. 281, 32774-32783 (2006).; 33. Lacey et al, ChemBioChem 14, 2100-2105 (2013).; 34. Takimoto et al, ACS Chem. Biol. 6, 733-743 (2011).; 35. Kobayashi et al, J. Am. Chem. Soc. 138, 14832-14835 (2016).; 36. Yamakawa, N. et al. Sci. Reports 10, 8647-14 (2020).; 37. Portoukalian et al, Eur. J. Biochem. 94, 19-23 (1979).; 38. Razi et al, Proc. Natl. Acad. Sci. U.S.A. 95, 7469-7474 (1998).; 39. Dippold, W. G. et al. Proc. Natl. Acad. Sci. U.S.A. 77, 6114-6118 (1980).; 40. Suck, G. et al. Cancer Immunol. Immunother. 65, 485-492 (2016).; 41. Hu, C.-W. et al. Nat. Chem. Biol. 13, 1267-1273 (2017).; 42. Chang, P. V. et al. Angew. Chem. Int. Ed. Engl. 48, 4030-4033 (2009).

### Supplemental References Cited in Experimental Procedures

1. Borrel, G. et al. BMC Genomics 15, 679 (2014); 2. Willis, J. C. W. & Chin, J. W. Nature Chem. 10, 831-837 (2018); 3. Zhou, H. et al. Org. Lett. 20, 812-815 (2018); 4. Yang, X. et al. J. Med. Chem. 62, 3539-3552 (2019); 5. Takimoto, J. K., Dellas, N., Noel, J. P. & Wang, L. Stereochemical basis for engineered pyrrolysyl-tRNA synthetase and the efficient in vivo incorporation of structurally divergent non-native amino acids. ACS Chem. Biol. 6, 733-743 (2011); 6. Lacey, V. K., Louie, G. V., Noel, J. P. & Wang, L. Expanding the library and substrate diversity of the pyrrolysyl-tRNA synthetase to incorporate unnatural amino acids containing conjugated rings. ChemBioChem 14, 2100-2105 (2013); 7. Wang, N. et al. Genetically encoding fluorosulfate-1-tyrosine to react with lysine, histidine, and tyrosine via SuFEx in proteins in vivo. J. Am. Chem. Soc. 140, 4995-4999 (2018); 8. Chen. Z.-L. et al. Nat. Commun. 10, 3404 (2019); 9. Liu. C. et al. Adv. Biol. (Weinh) 5, e2000308 (2021).

What is claimed is:

1. A compound of Formula (I): 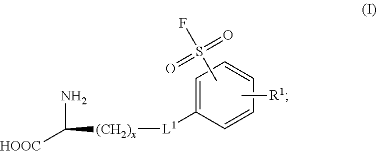
wherein:
x is an integer from 0 to 8;
L¹ is a bond, substituted or unsubstituted alkylene, or substituted or unsubstituted heteroalkylene;
R¹ is halogen, —CX¹₃, —CHX¹₂, —CH₂X¹, —OCX¹₃, —OCH₂X¹, —OCHX¹₂, —CN, —SOₙ₁R^{1A}, —SOᵥ₁NR^{1A}R^{1B}, —NHC(O)NR^{1A}R^{1B}, —N(O)ₘ₁, —NR^{1A}R^{1B}, —C(O)R^{1A}, —C(O)—OR^{1A}, —C(O)NR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, substituted or unsubstituted alkyl, or substituted or unsubstituted heteroalkyl;
X¹ is independently —F, —Cl, —Br, or —I;
R^{1A} is hydrogen, substituted or unsubstituted alkyl, or substituted or unsubstituted heteroalkyl;
R^{1B} is hydrogen, substituted or unsubstituted alkyl, or substituted or unsubstituted heteroalkyl;
n1 is an integer from 0 to 4
m¹ is 1 or 2; and
v1 is 1 or 2.

2. The compound of claim 1, wherein x is an integer from 1 to 4.

3. The compound of claim 1, wherein L¹ is a bond.

4. The compound of claim 1, wherein L¹ is substituted or unsubstituted 2 to 6 membered heteroalkylene.

5. The compound of claim 4, wherein L¹ is —NH—C(O)—(CH₂)_{y}— or —NH—C(O)—O—(CH₂)_{y}—, and y is an integer from 0 to 3.

6. The compound of claim 1, wherein R¹ is substituted or unsubstituted heteroalkyl.

7. The compound of claim 6, wherein R¹ is unsubstituted 2 to 8 membered heteroalkyl.

8. The compound of claim 7, wherein R¹ is —O—(CH₂)ₘCH₃, and m is an integer from 0 to 4.

9. The compound of claim 1, wherein R¹ is ortho to —S(═O)₂F.

10. The compound of claim 1, wherein the compound of Formula (I) is a compound of Formula (IA):

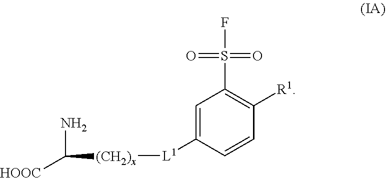

11. The compound of claim 1, wherein the compound of Formula (I) is a compound of Formula (IB):

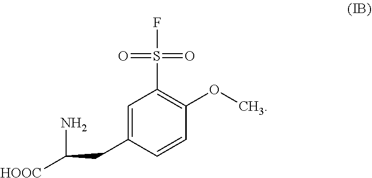 [SMILES: COc1ccc(C[C@H](N)C(=O)O)cc1S(=O)(=O)F]

12. A biomolecule comprising an unnatural amino acid, wherein the unnatural amino comprises a side chain of Formula (II): 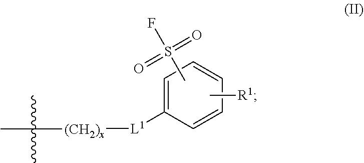
wherein:
x is an integer from 1 to 8;
L¹ is a bond, substituted or unsubstituted alkylene, or substituted or unsubstituted heteroalkylene;
R¹ is halogen, —CX¹₃, —CHX¹₂, —CH₂X¹, —OCX¹₃, —OCH₂X¹, —OCHX¹₂, —CN, —SOₙ₁R^{1A}, —SOᵥ₁NR^{1A}R^{1B}, —NHC(O)NR^{1A}R^{1B}, —N(O)ₘ₁, —NR^{1A}R^{1B}, —C(O)R^{1A}, —C(O)—OR^{1A}, —C(O)NR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, substituted or unsubstituted alkyl, or substituted or unsubstituted heteroalkyl;
X¹ is independently —F, —Cl, —Br, or —I;
R^{1A} is hydrogen, substituted or unsubstituted alkyl, or substituted or unsubstituted heteroalkyl;
R^{1B} is hydrogen, substituted or unsubstituted alkyl, or substituted or unsubstituted heteroalkyl;
n1 is an integer from 0 to 4
m¹ is 1 or 2; and
v1 is 1 or 2.

13. The biomolecule of claim 12, wherein x is an integer from 1 to 4.

14. The biomolecule of claim 12, wherein L¹ is a bond.

15. The biomolecule of claim 12, wherein L¹ is substituted or unsubstituted 2 to 6 membered heteroalkylene.

16. The biomolecule of claim 15, wherein L¹ is —NH—C(O)—(CH₂)_{y}— or —NH—C(O)—O—(CH₂)_{y}—, and y is an integer from 0 to 2.

17. The biomolecule of claim 12, wherein R¹ is substituted or unsubstituted heteroalkyl.

18. The biomolecule of claim 17, wherein R¹ is unsubstituted 2 to 8 membered heteroalkyl.

19. The biomolecule of claim 18, wherein R¹ is —O—(CH₂)ₘCH₃, and m is an integer from 0 to 4.

20. The biomolecule of claim 12, wherein R¹ is ortho to —S(═O)₂F.

21. The biomolecule of claim 12, wherein the side chain of Formula (II) has the structure of Formula (IIA):

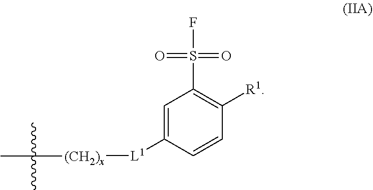

22. The biomolecule of claim 12, wherein the side chain of Formula (II) has the structure of Formula (IIB):

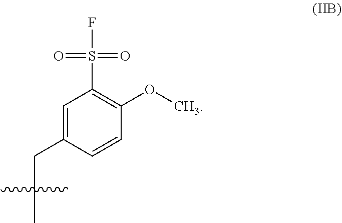 [SMILES: *Cc1ccc(OC)c(S(=O)(=O)F)c1]

23. The biomolecule of claim 12, wherein the biomolecule comprises a lipid or RNA.

24. The biomolecule of claim 12, wherein the biomolecule comprises a protein.

25. The biomolecule of claim 24, wherein the protein comprises a glycan-binding protein which comprises the unnatural amino acid.

26. The biomolecule of claim 25, wherein the glycan-binding protein is a sialic acid-binding immunoglobulin-type lectin (Siglec) which comprises the unnatural amino acid or a sialoglycan binding V-set domain of sialic acid-binding immunoglobulin-type lectin (Siglec) which comprises the unnatural amino acid.

27. The biomolecule of claim 26, wherein the Siglec is Siglec-1, Siglec-2, Siglec-3, Siglec-4, Siglec-5, Siglec-6, Siglec-7, Siglec-8, Siglec-9, Siglec-10, Siglec-11, Siglec-12, Siglec-14, or Siglec-15.

28. The biomolecule of claim 27, wherein the Siglec is Siglec-7.

29. The biomolecule of claim 28, wherein the Siglec-7 has at least 85% sequence identity to SEQ ID NO: 1, SEQ ID NO:2, SEQ ID NO:3, or SEQ ID NO:4.

30. The biomolecule of claim 26, wherein the side chain is at a lysine residue at a position corresponding to position 104 or position 127; or wherein the side chain is at an asparagine residue at a position corresponding to position 129.

31. The biomolecule of claim 24, wherein the protein is an RNA-binding protein which comprises the unnatural amino acid.

32. The biomolecule of claim 24, wherein the protein is a N⁶-methyladenosine reader protein which comprises the unnatural amino acid.

33. A nucleic acid encoding the biomolecule of claim 12.

34. A vector comprising the nucleic acid sequence of claim 33.

35. A biomolecule conjugate of Formula (III): 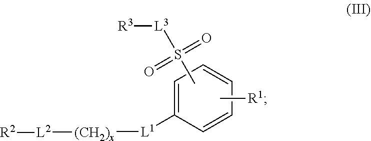
wherein:
R² is a first biomolecule moiety;
R³ is a second biomolecule moiety;
L¹ is a bond, substituted or unsubstituted alkylene, or substituted or unsubstituted heteroalkylene;
x is an integer from 1 to 8;
R¹ is halogen, —CX¹₃, —CHX¹₂, —CH₂X¹, —OCX¹₃, —OCH₂X¹, —OCHX¹₂, —CN, —SOₙ₁R^{1A}, —SOᵥ₁NR^{1A}R^{1B}, —NHC(O)NR^{1A}R^{1B}, —N(O)ₘ₁, —NR^{1A}R^{1B}, —C(O)R^{1A}, —C(O)—OR^{1A}, —C(O)NR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, substituted or unsubstituted alkyl, or substituted or unsubstituted heteroalkyl;
X¹ is independently —F, —Cl, —Br, or —I;
R^{1A} is hydrogen, substituted or unsubstituted alkyl, or substituted or unsubstituted heteroalkyl;
R^{1B} is hydrogen, substituted or unsubstituted alkyl, or substituted or unsubstituted heteroalkyl;
n1 is an integer from 0 to 4;
m¹ is 1 or 2;
v1 is 1 or 2;
L² is a bond, —NR^{2A}—, —S—, —S(O)₂—, —O—, —C(O)—, —C(O)O—, —OC(O)—, —N(R^{2A})C(O)—, —C(O)N(R^{2A})—, —NR^{2A}C(O)NR^{2B}—, —NR^{2A}C(NH)NR^{2B}—, —SO₂N(R^{2A})—, —N(R^{2A})SO₂—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L³ is a bond, —N(R^{3A})—, —S—, —S(O)₂—, —O—, —C(O)—, —C(O)O—, —OC(O)—, —N(R^{3A})C(O)—, —C(O)N(R^{3A})—, —NR^{3A}C(O)NR^{3B}—, —NR^{3A}C(NH)NR^{3B}—, —SO₂N(R^{3A})—, —N(R^{3A})SO₂—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
R^{2A}, R^{2B}, R^{3A}, and R^{3B} are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

36. The biomolecule conjugate of claim 35, wherein x is an integer from 1 to 4.

37. The biomolecule conjugate of claim 35, wherein L¹ is a bond.

38. The biomolecule conjugate of claim 35, wherein L¹ is substituted or unsubstituted 2 to 6 membered heteroalkylene.

39. The biomolecule conjugate of claim 38, wherein L¹ is —NH—C(O)—(CH₂)_{y}— or —NH—C(O)—O—(CH₂)_{y}—, and y is an integer from 0 to 2.

40. The biomolecule conjugate of claim 35, wherein R¹ is substituted or unsubstituted heteroalkyl.

41. The biomolecule conjugate of claim 40, wherein R¹ is unsubstituted 2 to 8 membered heteroalkyl.

42. The biomolecule conjugate of claim 41, wherein R¹ is —O—(CH₂)ₘCH₃, and m is an integer from 0 to 4.

43. The biomolecule conjugate of claim 35, wherein R¹ is ortho to —S(═O)₂F.

44. The biomolecule conjugate of claim 35, wherein:
L² is a bond, —NH—, —S—, —S(O)₂—, —O—, —C(O)—, —C(O)O—, —OC(O)—, —NHC(O)—, —C(O)NH—, —NHC(O)NH—, —NHC(NH)NH—, —SO₂NH—, —NHSO₂—, —C(S)—, L¹²-substituted or unsubstituted alkylene, L¹²-substituted or unsubstituted heteroalkylene, L¹²-substituted or unsubstituted cycloalkylene, L¹²-substituted or unsubstituted heterocycloalkylene, L¹²-substituted or unsubstituted arylene, or L¹²-substituted or unsubstituted heteroarylene;
L¹² is halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —N(O)₂, —NHSO₂H, —NHC(O)H, —NHC(O)OH, —NHOH, —N₃, unsubstituted alkyl, unsubstituted heteroalkyl, unsubstituted cycloalkyl, unsubstituted heterocycloalkyl, unsubstituted aryl, or unsubstituted heteroaryl;
L³ is a bond, —NH—, —S—, —S(O)₂—, —O—, —C(O)—, —C(O)O—, —OC(O)—, —NHC(O)—, —C(O)NH—, —NHC(O)NH—, —NHC(NH)NH—, —SO₂NH—, —NHSO₂—, —C(S)—, L¹³-substituted or unsubstituted alkylene, L¹³-substituted or unsubstituted heteroalkylene, L¹³-substituted or unsubstituted cycloalkylene, L¹³-substituted or unsubstituted heterocycloalkylene, L¹³-substituted or unsubstituted arylene, or L¹²-substituted or unsubstituted heteroarylene; and
L¹³ is halogen, —CF₃, —CBr₃, —CCl₃, —CI₃, —CHF₂, —CHBr₂, —CHCl₂, —CHI₂, —CH₂F, —CH₂Br, —CH₂Cl, —CH₂I, —OCF₃, —OCBr₃, —OCCl₃, —OCI₃, —OCHF₂, —OCHBr₂, —OCHCl₂, —OCHI₂, —OCH₂F, —OCH₂Br, —OCH₂Cl, —OCH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —N(O)₂, —NHSO₂H, —NHC(O)H, —NHC(O)OH, —NHOH, —N₃, unsubstituted alkyl, unsubstituted heteroalkyl, unsubstituted cycloalkyl, unsubstituted heterocycloalkyl, unsubstituted aryl, or unsubstituted heteroaryl.

45. The biomolecule conjugate of claim 35, wherein the biomolecule conjugate of Formula (III) is a biomolecule conjugate of Formula (IIIA):

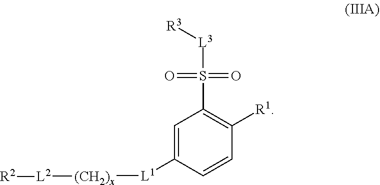

46. The biomolecule conjugate of claim 45, wherein the biomolecule conjugate of Formula (IIIA) is a biomolecule conjugate of Formula (IIIB):

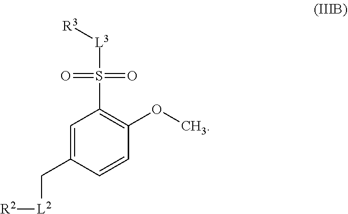

47. The biomolecule conjugate of claim 35, wherein L² is a bond and L³ is a bond.

48. The biomolecule conjugate of claim 35, wherein R² is a peptidyl moiety, a lipid moiety, an RNA moiety, or a glycan moiety, and R³ is a peptidyl moiety, a lipid moiety, a RNA moiety, or a glycan moiety.

49. The biomolecule conjugate of claim 35, wherein R² is a peptidyl moiety, a lipid moiety, or a glycan moiety, and R³ is a RNA moiety.

50. The biomolecule conjugate of claim 35, wherein R² is a peptidyl moiety, and R³ is a RNA moiety.

51. The biomolecule conjugate of claim 50, wherein the peptidyl moiety is a RNA-binding peptidyl moiety.

52. The biomolecule conjugate of claim 50, wherein the peptidyl moiety is a N⁶-methyladenosine reader protein moiety.

53. The biomolecule conjugate of claim 50, wherein L³ is bonded to a N⁶-methyladenosine residue on the RNA moiety.

54. The biomolecule conjugate of claim 53, wherein L³ is a bond.

55. The biomolecule conjugate of claim 35, wherein R² is a peptidyl moiety, a lipid moiety, or an RNA moiety, and R³ is a glycan moiety.

56. The biomolecule conjugate of claim 35, wherein R² is a peptidyl moiety and R³ is a glycan moiety.

57. The biomolecule conjugate of claim 56, wherein R² is a glycan-binding peptidyl moiety and R³ is a glycan moiety.

58. The biomolecule conjugate of claim 57, wherein the glycan-binding peptidyl moiety comprises a sialic acid-binding immunoglobulin-type lectin (Siglec) which comprises the unnatural amino acid; and wherein the glycan moiety comprises a sialoglycan.

59. The biomolecule conjugate of claim 57, wherein the peptidyl moiety comprises a sialoglycan binding V-set domain of sialic acid-binding immunoglobulin-type lectin (Siglec) which comprises the unnatural amino acid; and wherein the glycan moiety comprises a sialoglycan.

60. The biomolecule conjugate of claim 58, wherein the Siglec is Siglec-1, Siglec-2, Siglec-3, Siglec-4, Siglec-5, Siglec-6, Siglec-7, Siglec-8, Siglec-9, Siglec-10, Siglec-11, Siglec-12, Siglec-14, or Siglec-15.

61. The biomolecule conjugate of claim 60, wherein the Siglec is Siglec-7.

62. The biomolecule conjugate of claim 61, wherein Siglec-7 has at least 85% sequence identity to SEQ ID NO:1, SEQ ID NO:2, SEQ ID NO:3, or SEQ ID NO:4.

63. The biomolecule conjugate of claim 62, wherein R² is bonded to L² at a lysine residue at a position corresponding to position 104 or position 127; or wherein R² is bonded to L² at an asparagine residue at a position corresponding to position 129.

64. The biomolecule conjugate of claim 58, wherein the sialoglycan is bonded to L³ via an oxygen atom within the sialoglycan.

65. The biomolecule conjugate of claim 64, wherein L³ is a bond.

66. The biomolecule conjugate of claim 55, wherein the glycan moiety is further bonded to a lipid, a protein, or RNA.

67. The biomolecule conjugate of claim 66, wherein the glycan moiety is bonded to a cell membrane lipid.

68. The biomolecule conjugate of claim 67, wherein the cell membrane lipid is a cancer cell membrane lipid.

69. A pyrrolysyl-tRNA synthetase comprising at least 6 amino acid residues substitutions within the substrate-binding site of the pyrrolysyl-tRNA synthetase having at least 85% sequence identity to the amino acid sequence of SEQ ID NO:5; wherein the substrate-binding site comprises residues tyrosine at position 306, leucine at position 309, asparagine at position 346, cysteine at position 348, and tryptophan at position 417 as set forth in the amino acid sequence of SEQ ID NO:5:

70. The pyrrolysyl-tRNA synthetase of claim 69, wherein the at least 6 amino acid residues substitutions in the amino acid sequence of SEQ ID NO:5 are: (i) Y306L; (ii) L309A; (iii) N346A; (iv) C348M; and (v) W417T.

71. A pyrrolysyl-tRNA synthetase comprising at least 6 amino acid residues substitutions within the substrate-binding site of the pyrrolysyl-tRNA synthetase having at least 85% sequence identity to the amino acid sequence of SEQ ID NO:7; wherein the substrate-binding site comprises residues tyrosine at position 126, methionine at position 129, asparagine at position 166, valine at position 168, and tryptophan at position 239 as set forth in the amino acid sequence of SEQ ID NO:7:

72. The pyrrolysyl-tRNA synthetase of claim 71, wherein the at least 6 amino acid residues substitutions in the amino acid sequence of SEQ ID NO:7 are: (i) Y126L; (ii) M129A; (iii) N166A; (iv) V168M; and (v) W239T.

73. A nucleic acid encoding the pyrrolysyl-tRNA synthetase of claim 69.

74. A vector comprising the nucleic acid of claim 73.

75. The vector of claim 74, further comprising a nucleic acid encoding tRNA^{Pyl}.

76. A complex comprising the pyrrolysyl-tRNA synthetase of claim 69 and the compound of claim 1.

77. The complex of claim 76, further comprising a tRNA^{Pyl}.

78. A cell comprising: (i) the compound of any one of claims 1 to 11; (ii) the biomolecule of any one of claims 12 to 32; (iii) the nucleic acid of claim 33 or 73; (iv) the vector of claim 34, 74, or 75; (v) the biomolecule conjugate of any one of claims 35 to 68; (vi) the pyrrolysyl-tRNA synthetase of any one of claims 69 to 72; or (vii) the complex of claim 76 or 77.

79. The cell of claim 78, wherein the cell is a bacterial cell or a mammalian cell.

80. A pharmaceutical composition comprising the biomolecule of claim 12, the nucleic acid of claim 33, or the vector of claim 34, and a pharmaceutically acceptable excipient.

81. A method of treating cancer in a patient in need thereof, the method comprising administering to the patient an effective amount of the biomolecule of claim 12, the nucleic acid of claim 33, the vector of claim 34, or the pharmaceutical composition of claim 80.

82. The method of claim 77, comprising administering to the patient an effective amount of the biomolecule of claim 26.

83. The method of claim 81, wherein the cancer is melanoma or breast cancer.

84. The method of claim 81, wherein the cancer comprises a sialoglycan.

85. The method of claim 81, wherein the cancer comprises an elevated level of sialoglycan relative to a control.

86. The method of claim 81, further comprising detecting an elevated level of sialoglycan in a biological sample obtained from the patient.

87. A method of identifying a N⁶-methyladenosine site on RNA, the method comprising contacting the biomolecule of claim 32 with the RNA, thereby identify the N⁶-methyladenosine site.

88. A method of identifying a N⁶-methyladenosine site on RNA, the method comprising contacting the biomolecule of claim 24 with the RNA, wherein the protein is a N⁶-methyladenosine demethylase protein, thereby identifying the N⁶-methyladenosine site.

89. The method of claim 87, wherein the RNA is in the transcriptome.

90. The biomolecule of claim 24, wherein the protein is a N⁶-methyladenosine demethylase protein which comprises the unnatural amino acid.
